(19) United States
(12) Patent Application Publication
(10) Pub. No.: US 2024/0025905 A1
(43) Pub. Date: Jan. 25, 2024

(54) SALTS OF DIHYDROPYRIMIDINE DERIVATIVES, COMPLEXES AND USES THEREOF IN MEDICINE

(71) Applicant: SUNSHINE LAKE PHARMA CO., LTD., Dongguan, Guangdong (CN)

(72) Inventors: Qingyun REN, Dongguan (CN); Xinchang LIU, Dongguan (CN); Yingjun ZHANG, Dongguan (CN); Guanghua YAN, Dongguan (CN)

(73) Assignee: SUNSHINE LAKE PHARMA CO., LTD., Dongguan, Guangdong (CN)

(21) Appl. No.: 18/037,105

(22) PCT Filed: Nov. 30, 2021
(86) PCT No.: PCT/CN2021/134233

§ 371 Date: May 16, 2023

(30) Foreign Application Priority Data
Nov. 30, 2020 (CN) 202011379310.0

(51) Int. Cl. C07D 487/04

(52) U.S. Cl. CPC C07D 487/04

(57) ABSTRACT

Salts of dihydropyrimidine derivatives, complexes and uses thereof in medicine, and complexes and pharmaceutical compositions thereof. The uses of the addition salts, the complexes or the pharmaceutical compositions in the manufacture of a medicament, especially in the manufacture of a medicament for preventing, managing, treating or lessening hepatitis B virus (HBV) infection.

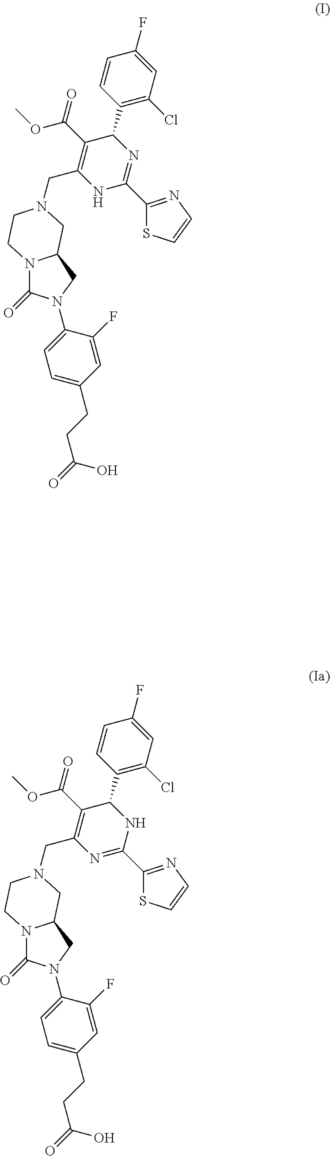 [SMILES: COC(=O)C1=C(CN2CCN3C(=O)N(c4ccc(CCC(=O)O)cc4F)C[C@@H]3C2)NC(c2nccs2)=N[C@H]1c1ccc(F)cc1Cl ; COC(=O)C1=C(CN2CCN3C(=O)N(c4ccc(CCC(=O)O)cc4F)C[C@@H]3C2)N=C(c2nccs2)N[C@H]1c1ccc(F)cc1Cl]

### CROSS-REFERENCE TO RELATED APPLICATION

This application claims the priority and benefits of Chinese Patent Application No. 202011379310.0, filed with the State Intellectual Property Office of China on Nov. 30, 2020, which is incorporated herein by reference in its entirety.

### FIELD

The invention belongs to the field of medicine, and the invention specifically relates to a compound 3-(4-((S)-7-(((R)-6-(2-chloro-4-fluorophenyl)-5-(methoxycarbonyl)-2-(thiazol-2-yl)-3,6-dihydropyrimidin-4-yl)methyl)-3-oxohexahydroimidazo[1,5-a]pyrazine-2(3H)-yl)-3-fluorophenyl)propionic acid (I) or various solid forms of its tautomer 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid (Ia), such as salts, complexes and pharmaceutical compositions thereof. The invention further relates to the use of the solid forms and pharmaceutical compositions thereof in the manufacture of a medicament, especially in the manufacture of a medicament for preventing, managing, treating or lessening hepatitis B virus (HBV) infection.

### BACKGROUND

The hepatitis B virus belongs to the family of hepadnaviridae. It can cause acute and/or persistent or progressive chronic diseases. Many other clinical manifestations in the pathological morphology can be also caused by HBV—in particular chronic hepatitis, cirrhosis and hepatocellular carcinoma. Additionally, coinfection with hepatitis D virus may have adverse effects on the progress of the disease.

PCT application WO2019076310A1 discloses a compound having Formula (I) or (Ia) and a preparation method thereof. The compound having Formula (I) or (Ia) has good HBV inhibitory activity.

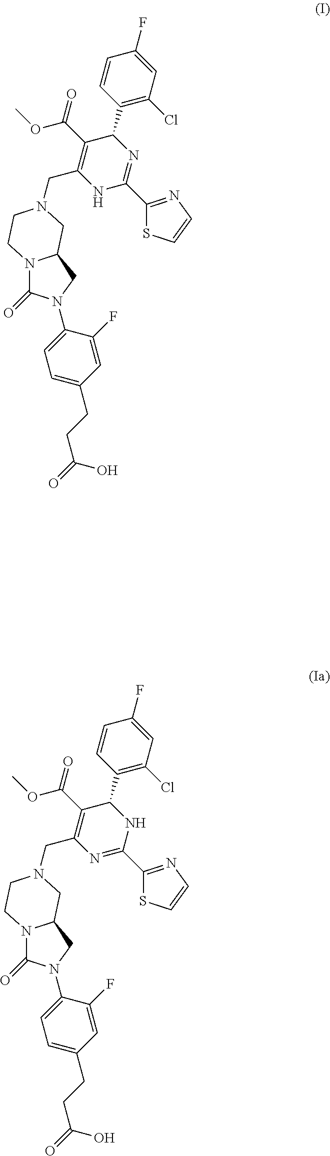 [SMILES: COC(=O)C1=C(CN2CCN3C(=O)N(c4ccc(CCC(=O)O)cc4F)C[C@@H]3C2)NC(c2nccs2)=N[C@H]1c1ccc(F)cc1Cl ; COC(=O)C1=C(CN2CCN3C(=O)N(c4ccc(CCC(=O)O)cc4F)C[C@@H]3C2)N=C(c2nccs2)N[C@H]1c1ccc(F)cc1Cl]

In the process of preparing the compound 3-(4-((S)-7-(((R)-6-(2-chloro-4-fluorophenyl)-5-(methoxycarbonyl)-2-(thiazol-2-yl)-3,6-dihydropyrimidin-4-yl)methyl)-3-oxohexahydroimidazo[1,5-a]pyrazine-2(3H)-yl)-3-fluorophenyl)propionic acid (I) and its tautomers 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid (Ia), it has been found that the stability of the compound is unsatisfactory (Especially unstable under high temperature (such as 60° C.)), which is not conducive to storage and weighing, and the absorption in the body is also unsatisfactory. These shortcomings bring many inconveniences to the subsequent formulation development.

Different salts or solid forms of active pharmaceutical ingredients may have different properties. Changes in the properties of different salts or solid forms can provide improved formulations, for example, ease of synthesis or handling, improving dissolution rate, stability and shelf life. The property changes caused by different salts or solid forms can also improve the pharmacological properties of the final formulation products, for example, increasing exposure, bioavailabiity or prolonging half-life.

### SUMMARY OF THE INVENTION

In order to find a solid form with better medicinal properties, through a large number of experimental studies, the inventors found that the salt of the compound having Formula (I) or tautomer (Ia) or the complex thereof is very stable under high temperature, high humidity and light conditions, and also has good pharmacokinetic properties, such as high exposure, good absorption and low hygroscopicity.

The present invention provides salts of compound 3-(4-((S)-7-(((R)-6-(2-chloro-4-fluorophenyl)-5-(methoxycarbonyl)-2-(thiazol-2-yl)-3,6-dihydropyrimidin-4-yl)methyl)-3-oxohexahydroimidazo[1,5-a]pyrazine-2(3H)-yl)-3-fluorophen yl)propionic acid (I) or its tautomer (Ia), complexes, and pharmaceutical compositions containing them. The present invention further provides a use of the salts, complexes and pharmaceutical compositions in the manufacture of a medicament, especially in the manufacture of a medicament for preventing, managing, treating or lessening hepatitis B virus (HBV) infection.

In one aspect, provided herein is a salt of a compound having Formula (I) or (Ia),

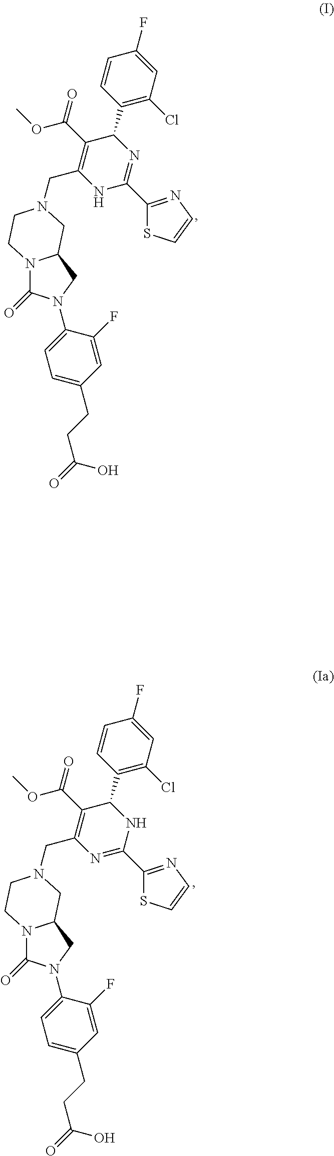

wherein, the salt is sulfate, L-arginine salt, hydrochloride, phosphate, benzenesulfonate, methanesulfonate, hydrobromide, p-toluenesulfonate or oxalate.

In some embodiments, the sulfate of the present invention is sulfate crystal form B, and the X-ray powder diffraction pattern of the sulfate crystal form B comprises diffraction peaks with 2θ angles of 6.02±0.2°, 16.74±0.2°, 17.34±0.2°, 18.17±0.2°, 19.52±0.2° and 24.32±0.2°.

In some embodiments, the sulfate of the present invention is sulfate crystal form B, and the X-ray powder diffraction pattern of the sulfate crystal form B comprises diffraction peaks with 2θ angles of 6.02±0.2°, 13.70±0.2°, 16.74±0.2°, 17.34±0.2°, 18.17±0.2°, 19.52±0.2°, 23.72±0.2°, 24.32±0.2°, 24.68±0.2° and 25.91±0.2°.

In some embodiments, the sulfate of the present invention is sulfate crystal form B, and the X-ray powder diffraction pattern of the sulfate crystal form B comprises diffraction peaks with 2θ angles of 6.02±0.2°, 9.05±0.2°, 11.28±0.2°, 12.09±0.2°, 12.68±0.2°, 13.70±0.2°, 14.17±0.2°, 15.27±0.2°, 16.29±0.2°, 16.49±0.2°, 16.74±0.2°, 17.34±0.2°, 17.56±0.2°, 18.17±0.2°, 18.69±0.2°, 19.52±0.2°, 20.47±0.2°, 21.24±0.2°, 21.87±0.2°, 22.48±0.2°, 22.71±0.2°, 23.72±0.2°, 24.32±0.2°, 24.68±0.2°, 24.82±0.2°, 25.35±0.2°, 25.91±0.2°, 26.77±0.2°, 27.36±0.2°, 27.99±0.2°, 28.64±0.2°, 29.51±0.2°, 29.85±0.2°, 30.19±0.2°, 30.55±0.2°, 31.23±0.2°, 32.21±0.2°, 33.09±0.2°, 33.68±0.2°, 34.85±0.2°, 35.46±0.2°, 36.84±0.2°, 37.43±0.2°, 39.06±0.2° and 39.96±0.2°.

In some embodiments, the L-arginine salt of the present invention is L-arginine salt crystal form A, and the X-ray powder diffraction pattern of the L-arginine salt crystal form A comprises diffraction peaks with 2θ angles of 10.50±0.2°, 12.52±0.2°, 16.88±0.2°, 19.30±0.2°, 20.29±0.2°, 20.61±0.2° and 23.04±0.2°.

In some embodiments, the L-arginine salt of the present invention is L-arginine salt crystal form A, and the X-ray powder diffraction pattern of the L-arginine salt crystal form A comprises diffraction peaks with 2θ angles of 10.50±0.2°, 12.52±0.2°, 13.52±0.2°, 16.88±0.2°, 17.07±0.2°, 19.30±0.2°, 20.29±0.2°, 20.61±0.2°, 23.04±0.2° and 28.54±0.2°.

In some embodiments, the L-arginine salt of the present invention is L-arginine salt crystal form A, and the X-ray powder diffraction pattern of the L-arginine salt crystal form A comprises diffraction peaks with 2θ angles of 8.50±0.2°, 10.50±0.2°, 12.52±0.2°, 12.71±0.2°, 13.05±0.2°, 13.52±0.2°, 14.23±0.2°, 15.76±0.2°, 16.60±0.2°, 16.88±0.2°, 17.07±0.2°, 18.22±0.2°, 19.11±0.2°, 19.30±0.2°, 19.58±0.2°, 20.29±0.2°, 20.61±0.2°, 20.98±0.2°, 22.53±0.2°, 23.04±0.2°, 24.90±0.2°, 25.41±0.2°, 25.68±0.2°, 26.11±0.2°, 26.68±0.2°, 27.22±0.2°, 28.07±0.2°, 28.29±0.2°, 28.54±0.2°, 30.12±0.2°, 31.06±0.2°, 31.68±0.2°, 33.55±0.2°, 34.50±0.2°, 34.89±0.2°, 35.24±0.2°, 36.12±0.2°, 36.65±0.2°, 38.68±0.2° and 39.80±0.2°.

In some embodiments, the hydrochloride of the present invention is hydrochloride crystal form A, and the X-ray powder diffraction pattern of the hydrochloride crystal form A comprises diffraction peaks with 2θ angles of 10.94±0.2°, 11.82±0.2°, 16.64±0.2°, 19.22±0.2°, 19.64±0.2, 23.44±0.2°, 24.89±0.2° and 26.08±0.2°.

In some embodiments, the hydrochloride of the present invention is hydrochloride crystal form A, and the X-ray powder diffraction pattern of the hydrochloride crystal form A comprises diffraction peaks with 2θ angles of 10.94±0.2°, 11.28±0.2°, 11.82±0.2°, 12.08±0.2°, 16.64±0.2°, 19.22±0.2°, 19.64±0.2°, 20.46±0.2°, 23.44±0.2°, 24.89±0.2°, 26.08±0.2° and 28.65±0.2°.

In some embodiments, the hydrochloride of the present invention is hydrochloride crystal form A, and the X-ray powder diffraction pattern of the hydrochloride crystal form A comprises diffraction peaks with 2θ angles of 10.94±0.2°, 11.28±0.2°, 11.82±0.2°, 12.08±0.2°, 12.57±0.2°, 14.06±0.2°, 15.01±0.2°, 15.81±0.2°, 16.02±0.2°, 16.64±0.2°, 17.18±0.2°, 17.86±0.2°, 18.55±0.2°, 19.22±0.2°, 19.64±0.2°, 20.46±0.2°, 21.41±0.2°, 22.19±0.2°, 23.44±0.2°, 23.85±0.2°, 24.28±0.2°, 24.89±0.2°, 25.25±0.2°, 26.08±0.2°, 26.37±0.2°, 27.09±0.2°, 27.53±0.2°, 28.00±0.2°, 28.65±0.2°, 28.91±0.2°, 30.53±0.2°, 31.42±0.2°, 31.92±0.2°, 32.40±0.2°, 33.58±0.2°, 34.36±0.2°, 35.38±0.2°, 36.07±0.2°, 37.39±0.2° and 38.58±0.2°.

In some embodiments, the sulfate of the present invention is sulfate crystal form B, and the sulfate crystal form B has an X-ray powder diffraction pattern substantially as shown in FIG. 1.

In some embodiments, the L-arginine salt of the present invention is L-arginine salt crystal form A, and the L-arginine salt crystal form A has an X-ray powder diffraction pattern substantially as shown in FIG. 3.

In some embodiments, the hydrochloride of the present invention is hydrochloride crystal form A, and the hydrochloride crystal form A has an X-ray powder diffraction pattern substantially as shown in FIG. 5.

In some embodiments, the sulfate of the present invention is sulfate crystal form B, and the differential scanning calorimetry pattern of the sulfate crystal form B comprises an endothermic peak of 227.14° C.±3° C.

In some embodiments, the L-arginine salt of the present invention is L-arginine salt crystal form A, and the differential scanning calorimetry pattern of the L-arginine salt crystal form A comprises an endothermic peak of 193.28° C.±3° C.

In some embodiments, the hydrochloride of the present invention is hydrochloride crystal form A, and the differential scanning calorimetry pattern of the hydrochloride crystal form A comprises endothermic peaks of 134.08° C.±3° C. and 176.08° C.±3° C.

In some embodiments, the sulfate is sulfate crystal form B, and the sulfate crystal form B has a differential scanning calorimetry pattern substantially as shown in FIG. 2.

In some embodiments, the L-arginine salt is L-arginine salt crystal form A, and the L-arginine salt crystal form A has a differential scanning calorimetry pattern substantially as shown in FIG. 4.

In some embodiments, the hydrochloride is hydrochloride crystal form A, and the hydrochloride crystal form A has a differential scanning calorimetry pattern substantially as shown in FIG. 6.

In some embodiments, the sulfate of the present invention is sulfate crystal form A, and the X-ray powder diffraction pattern of the sulfate crystal form A comprises diffraction peaks with 2θ angles of 5.74±0.2°, 8.62±0.2°, 10.52±0.2°, 13.97±0.2°, 17.75±0.2°, 19.28±0.2°, 23.38±0.2° and 24.78±0.2°.

In some embodiments, the sulfate of the present invention is sulfate crystal form A, and the X-ray powder diffraction pattern of the sulfate crystal form A comprises diffraction peaks with 2θ angles of 5.74±0.2°, 8.62±0.2°, 10.52±0.2°, 13.04±0.2°, 13.97±0.2°, 17.75±0.2°, 19.28±0.2°, 23.38±0.2°, 24.78±0.2°, 25.13±0.2° and 25.76±0.2°.

In some embodiments, the sulfate of the present invention is sulfate crystal form A, and the X-ray powder diffraction pattern of the sulfate crystal form A comprises diffraction peaks with 2θ angles of 5.74±0.2°, 8.62±0.2°, 10.52±0.2°, 11.08±0.2°, 13.04±0.2°, 13.97±0.2°, 14.42±0.2°, 15.40±0.2°, 16.11±0.2°, 16.56±0.2°, 17.25±0.2°, 17.75±0.2°, 18.38±0.2°, 19.28±0.2°, 19.74±0.2°, 21.14±0.2°, 21.57±0.2°, 22.33±0.2°, 23.38±0.2°, 24.78±0.2°, 25.13±0.2°, 25.76±0.2°, 26.31±0.2°, 26.80±0.2°, 27.12±0.2°, 27.83±0.2°, 28.08±0.2°, 29.32±0.2°, 30.45±0.2°, 31.31±0.2°, 31.87±0.2°, 33.08±0.2°, 34.87±0.2°, 36.01±0.2°, 36.95±0.2°, 37.42±0.2°, 38.59±0.2°, 39.03±0.2° and 39.92±0.2°.

In some embodiments, the sulfate of the present invention is sulfate crystal form A, and the sulfate crystal form A has an X-ray powder diffraction pattern substantially as shown in FIG. 9.

In some embodiments, the sulfate of the present invention is sulfate crystal form A, and the differential scanning calorimetry pattern of the sulfate crystal form A comprises an endothermic peak of 208.32° C.±3° C.

In some embodiments, the sulfate of the present invention is sulfate crystal form A, and the differential scanning calorimetry pattern of the sulfate crystal form A comprises endothermic peaks of 96.43° C.±3° C. and 208.32° C.±3° C.

In some embodiments, the sulfate of the present invention is sulfate crystal form A, and the sulfate crystal form A has a differential scanning calorimetry pattern substantially as shown in FIG. 10.

In some embodiments, the phosphate of the present invention is phosphate crystal form A, and the X-ray powder diffraction pattern of the phosphate crystal form A comprises diffraction peaks with 2θ angles of 6.01±0.2°, 13.76±0.2°, 15.95±0.2°, 16.75±0.2°, 23.52±0.2°, 24.14±0.2° and 24.72±0.2°.

In some embodiments, the phosphate of the present invention is phosphate crystal form A, and the X-ray powder diffraction pattern of the phosphate crystal form A comprises diffraction peaks with 2θ angles of 6.01±0.2°, 12.01±0.2°, 13.07±0.2°, 13.76±0.2°, 15.95±0.2°, 16.75±0.2°, 18.11±0.2°, 23.52±0.2°, 24.14±0.2° and 24.72±0.2°.

In some embodiments, the phosphate of the present invention is phosphate crystal form A, and the X-ray powder diffraction pattern of the phosphate crystal form A comprises diffraction peaks with 2θ angles of 6.01±0.2°, 10.88±0.2°, 12.01±0.2°, 13.07±0.2°, 13.76±0.2°, 13.88±0.2°, 14.99±0.2°, 15.64±0.2°, 15.95±0.2°, 16.75±0.2°, 18.11±0.2°, 18.37±0.2°, 18.99±0.2°, 19.76±0.2°, 20.94±0.2°, 21.16±0.2°, 21.48±0.2°, 21.78±0.2°, 22.82±0.2°, 23.52±0.2°, 24.14±0.2°, 24.72±0.2°, 25.03±0.2°, 25.63±0.2°, 25.80±0.2°, 26.34±0.2°, 26.83±0.2°, 27.15±0.2°, 28.49±0.2°, 28.90±0.2°, 29.21±0.2°, 29.61±0.2°, 30.02±0.2°, 31.55±0.2°, 32.04±0.2°, 33.37±0.2°, 33.87±0.2°, 34.36±0.2°, 35.06±0.2°, 35.42±0.2°, 35.86±0.2°, 36.53±0.2°, 36.91±0.2°, 37.67±0.2°, 38.48±0.2° and 39.91±0.2°.

In some embodiments, the phosphate of the present invention is phosphate crystal form A, and the phosphate crystal form A has an X-ray powder diffraction pattern substantially as shown in FIG. 11.

In some embodiments, the phosphate of the present invention is phosphate crystal form A, and the differential scanning calorimetry pattern of the phosphate crystal form A comprises an endothermic peak of 145.36° C.±3° C.

In some embodiments, the phosphate of the present invention is phosphate crystal form A, and the phosphate crystal form A has a differential scanning calorimetry pattern substantially as shown in FIG. 12.

In some embodiments, the phosphate of the present invention is phosphate crystal form B, and the X-ray powder diffraction pattern of the phosphate crystal form B comprises diffraction peaks with 2θ angles of 13.37±0.2°, 14.55±0.2°, 17.01±0.2°, 18.84±0.2°, 21.03±0.2° and 22.83±0.2°.

In some embodiments, the phosphate of the present invention is phosphate crystal form B, and the X-ray powder diffraction pattern of the phosphate crystal form B comprises diffraction peaks with 2θ angles of 13.37±0.2°, 14.55±0.2°, 17.01±0.2°, 18.04±0.2°, 18.84±0.2°, 21.03±0.2°, 22.83±0.2°, 23.83±0.2° and 25.80±0.2°.

In some embodiments, the phosphate of the present invention is phosphate crystal form B, and the X-ray powder diffraction pattern of the phosphate crystal form B comprises diffraction peaks with 2θ angles of 13.37±0.2°, 14.55±0.2°, 17.01±0.2°, 18.04±0.2°, 18.84±0.2°, 20.25±0.2°, 21.03±0.2°, 22.21±0.2°, 22.83±0.2°, 23.83±0.2°, 24.51±0.2°, 25.80±0.2°, 27.94±0.2°, 29.18±0.2°, 31.43±0.2°, 32.45±0.2° and 36.09±0.2°.

In some embodiments, the phosphate of the present invention is phosphate crystal form B, and the phosphate crystal form B has an X-ray powder diffraction pattern substantially as shown in FIG. 13.

In some embodiments, the phosphate of the present invention is phosphate crystal form B, and the differential scanning calorimetry pattern of the phosphate crystal form B comprises endothermic peaks of 104.50° C.±3° C. and 137.94° C.±3° C.

In some embodiments, the phosphate of the present invention is phosphate crystal form B, and the phosphate crystal form B has a differential scanning calorimetry pattern substantially as shown in FIG. 14.

In some embodiments, the methanesulfonate of the present invention is methanesulfonate crystal form A, the X-ray powder diffraction pattern of the methanesulfonate crystal form A comprises diffraction peaks with 2θ angles of 5.34±0.2°, 7.82±0.2°, 14.89±0.2°, 16.62±0.2°, 19.39±0.2°, 22.41±0.2°, 23.25±0.2° and 24.08±0.2°.

In some embodiments, the methanesulfonate of the present invention is methanesulfonate crystal form A, the X-ray powder diffraction pattern of the methanesulfonate crystal form A comprises diffraction peaks with 2θ angles of 5.34±0.2°, 6.29±0.2°, 7.82±0.2°, 11.46±0.2°, 14.89±0.2°, 16.08±0.2°, 16.62±0.2°, 19.39±0.2°, 22.41±0.2°, 23.25±0.2° and 24.08±0.2°.

In some embodiments, the methanesulfonate of the present invention is methanesulfonate crystal form A, the X-ray powder diffraction pattern of the methanesulfonate crystal form A comprises diffraction peaks with 2θ angles of 5.34±0.2°, 6.29±0.2°, 7.82±0.2°, 10.73±0.2°, 11.46±0.2°, 11.78±0.2°, 12.67±0.2°, 14.12±0.2°, 14.89±0.2°, 15.77±0.2°, 16.08±0.2°, 16.62±0.2°, 17.19±0.2°, 17.49±0.2°, 18.04±0.2°, 18.51±0.2°, 18.96±0.2°, 19.39±0.2°, 19.78±0.2°, 20.28±0.2°, 21.46±0.2°, 21.64±0.2°, 21.85±0.2°, 22.41±0.2°, 23.25±0.2°, 23.72±0.2°, 24.08±0.2°, 25.53±0.2°, 25.80±0.2°, 26.23±0.2°, 26.60±0.2°, 27.01±0.2°, 27.68±0.2°, 27.69±0.2°, 28.18±0.2°, 28.66±0.2°, 29.51±0.2°, 29.80±0.2°, 30.07±0.2°, 31.04±0.2°, 32.19±0.2°, 32.77±0.2°, 33.23±0.2°, 33.91±0.2°, 34.87±0.2°, 36.49±0.2°, 37.30±0.2°, 38.09±0.2°, 38.36±0.2°, 38.85±0.2°, 39.50±0.2° and 39.83±0.2°.

In some embodiments, the methanesulfonate of the present invention is methanesulfonate crystal form A, and the methanesulfonate crystal form A has an X-ray powder diffraction pattern substantially as shown in FIG. 15.

In some embodiments, the methanesulfonate of the present invention is methanesulfonate crystal form A, and the differential scanning calorimetry pattern of the methanesulfonate crystal form A comprises endothermic peaks of 115.67° C.±3° C. and 175.40° C.±3° C.

In some embodiments, the methanesulfonate of the present invention is methanesulfonate crystal form A, and the methanesulfonate crystal form A has a differential scanning calorimetry pattern substantially as shown in FIG. 16.

In some embodiments, the p-toluenesulfonate of the present invention is p-toluenesulfonate crystal form A, the X-ray powder diffraction pattern of the p-toluenesulfonate crystal form A comprises diffraction peaks with 2θ angles of 5.57±0.2°, 10.46±0.2°, 12.08±0.2°, 16.15±0.2°, 18.30±0.2°, 23.70±0.2° and 24.37±0.2°.

In some embodiments, the p-toluenesulfonate of the present invention is p-toluenesulfonate crystal form A, the X-ray powder diffraction pattern of the p-toluenesulfonate crystal form A comprises diffraction peaks with 2θ angles of 5.57±0.2°, 10.46±0.2°, 12.08±0.2°, 12.84±0.2°, 15.79±0.2°, 16.15±0.2°, 18.30±0.2°, 20.59±0.2°, 23.70±0.2°, 24.15±0.2° and 24.37±0.2°.

In some embodiments, the p-toluenesulfonate of the present invention is p-toluenesulfonate crystal form A, the X-ray powder diffraction pattern of the p-toluenesulfonate crystal form A comprises diffraction peaks with 2θ angles of 5.57±0.2°, 10.46±0.2°, 11.10±0.2°, 12.08±0.2°, 12.84±0.2°, 14.46±0.2°, 15.79±0.2°, 16.15±0.2°, 17.01±0.2°, 17.44±0.2°, 18.30±0.2°, 18.85±0.2°, 20.59±0.2°, 21.92±0.2°, 22.53±0.2°, 22.98±0.2°, 23.70±0.2°, 24.15±0.2°, 24.37±0.2°, 25.20±0.2°, 25.43±0.2°, 25.91±0.2°, 26.20±0.2°, 26.79±0.2°, 27.06±0.2°, 27.51±0.2°, 28.12±0.2°, 30.07±0.2°, 31.10±0.2°, 31.75±0.2°, 33.44±0.2°, 34.04±0.2°, 36.05±0.2°, 37.21±0.2° and 39.52±0.2°.

In some embodiments, the p-toluenesulfonate of the present invention is p-toluenesulfonate crystal form A, and the p-toluenesulfonate crystal form A has an X-ray powder diffraction pattern substantially as shown in FIG. 17.

In some embodiments, the p-toluenesulfonate of the present invention is p-toluenesulfonate crystal form A, and the differential scanning calorimetry pattern of the p-toluenesulfonate crystal form A comprises endothermic peaks of 139.10° C.±3° C. and 186.22° C.±3° C.

In some embodiments, the p-toluenesulfonate of the present invention is p-toluenesulfonate crystal form A, and the p-toluenesulfonate crystal form A has a differential scanning calorimetry pattern substantially as shown in FIG. 18.

In some embodiments, the benzenesulfonate of the present invention is benzenesulfonate crystal form A, the X-ray powder diffraction pattern of the benzenesulfonate crystal form A comprises diffraction peaks with 2θ angles of 5.59±0.2°, 12.55±0.2°, 13.27±0.2°, 15.68±0.2°, 15.93±0.2°, 17.44±0.2°, 24.02±0.2° and 25.88±0.2°.

In some embodiments, the benzenesulfonate of the present invention is benzenesulfonate crystal form A, the X-ray powder diffraction pattern of the benzenesulfonate crystal form A comprises diffraction peaks with 2θ angles of 5.59±0.2°, 11.04±0.2°, 12.55±0.2°, 13.27±0.2°, 15.68±0.2°, 15.93±0.2°, 17.44±0.2°, 19.61±0.2°, 24.02±0.2°, 23.55±0.2° and 25.88±0.2°.

In some embodiments, the benzenesulfonate of the present invention is benzenesulfonate crystal form A, the X-ray powder diffraction pattern of the benzenesulfonate crystal form A comprises diffraction peaks with 2θ angles of 5.59±0.2°, 10.58±0.2°, 11.04±0.2°, 12.15±0.2°, 12.55±0.2°, 13.27±0.2°, 13.78±0.2°, 14.21±0.2°, 15.68±0.2°, 15.93±0.2°, 16.24±0.2°, 16.68±0.2°, 17.44±0.2°, 17.84±0.2°, 18.50±0.2°, 19.39±0.2°, 19.61±0.2°, 19.88±0.2°, 20.59±0.2°, 21.22±0.2°, 21.98±0.2°, 22.75±0.2°, 22.89±0.2°, 23.55±0.2°, 23.88±0.2°, 24.02±0.2°, 24.22±0.2°, 24.51±0.2°, 24.89±0.2°, 25.36±0.2°, 25.63±0.2°, 25.88±0.2°, 26.50±0.2°, 27.05±0.2°, 27.84±0.2°, 29.07±0.2°, 29.79±0.2°, 30.40±0.2°, 31.24±0.2°, 31.79±0.2°, 32.36±0.2°, 32.77±0.2°, 33.22±0.2°, 33.75±0.2°, 34.31±0.2°, 34.95±0.2°, 35.40±0.2°, 35.88±0.2°, 36.46±0.2°, 37.93±0.2°, 39.08±0.2°, 39.47±0.2° and 39.91±0.2°.

In some embodiments, the benzenesulfonate of the present invention is benzenesulfonate crystal form A, and the benzenesulfonate crystal form A has an X-ray powder diffraction pattern substantially as shown in FIG. 19.

In some embodiments, the benzenesulfonate of the present invention is benzenesulfonate crystal form A, and the differential scanning calorimetry pattern of the benzenesulfonate crystal form A comprises endothermic peaks of 116.64° C.±3° C. and 177.99° C.±3° C.

In some embodiments, the benzenesulfonate of the present invention is benzenesulfonate crystal form A, and the benzenesulfonate crystal form A has a differential scanning calorimetry pattern substantially as shown in FIG. 20.

In some embodiments, the hydrobromide of the present invention is hydrobromide crystal form A, the X-ray powder diffraction pattern of the hydrobromide crystal form A comprises diffraction peaks with 2θ angles of 6.34±0.2°, 12.03±0.2°, 15.85±0.2°, 19.67±0.2°, 21.37±0.2°, 23.33±0.2° and 25.92±0.2°.

In some embodiments, the hydrobromide of the present invention is hydrobromide crystal form A, the X-ray powder diffraction pattern of the hydrobromide crystal form A comprises diffraction peaks with 2θ angles of 6.34±0.2°, 12.03±0.2°, 15.85±0.2°, 16.58±0.2°, 19.67±0.2°, 20.45±0.2°, 21.37±0.2°, 23.33±0.2°, 24.74±0.2° and 25.92±0.2°.

In some embodiments, the hydrobromide of the present invention is hydrobromide crystal form A, the X-ray powder diffraction pattern of the hydrobromide crystal form A comprises diffraction peaks with 2θ angles of 6.34±0.2°, 9.50±0.2°, 11.25±0.2°, 12.03±0.2°, 12.54±0.2°, 14.05±0.2°, 15.46±0.2°, 15.85±0.2°, 16.58±0.2°, 17.13±0.2°, 17.87±0.2°, 18.50±0.2°, 19.28±0.2°, 19.67±0.2°, 20.45±0.2°, 21.37±0.2°, 22.31±0.2°, 23.33±0.2°, 23.75±0.2°, 24.74±0.2°, 25.09±0.2°, 25.92±0.2°, 26.15±0.2°, 26.48±0.2°, 26.98±0.2°, 27.44±0.2°, 28.09±0.2°, 28.70±0.2°, 29.24±0.2°, 30.35±0.2°, 31.29±0.2°, 31.98±0.2°, 32.27±0.2°, 32.77±0.2°, 35.37±0.2°, 35.88±0.2°, 37.25±0.2°, 38.44±0.2° and 39.93±0.2°.

In some embodiments, the hydrobromide of the present invention is hydrobromide crystal form A, and the hydrobromide crystal form A has an X-ray powder diffraction pattern substantially as shown in FIG. 21.

In some embodiments, the hydrobromide of the present invention is hydrobromide crystal form A, and the differential scanning calorimetry pattern of the hydrobromide crystal form A comprises endothermic peaks of 120.25° C.±3° C. and 194.76° C.±3° C.

In some embodiments, the hydrobromide of the present invention is hydrobromide crystal form A, and the hydrobromide crystal form A has a differential scanning calorimetry pattern substantially as shown in FIG. 22.

In some embodiments, the hydrochloride of the present invention is hydrochloride crystal form B, the X-ray powder diffraction pattern of the hydrochloride crystal form B comprises diffraction peaks with 2θ angles of 6.38±0.2°, 11.37±0.2°, 18.28±0.2°, 19.20±0.2°, 20.59±0.2°, 22.88±0.2° and 24.32±0.2°.

In some embodiments, the hydrochloride of the present invention is hydrochloride crystal form B, the X-ray powder diffraction pattern of the hydrochloride crystal form B comprises diffraction peaks with 2θ angles of 6.38±0.2°, 11.37±0.2°, 12.73±0.2°, 18.28±0.2°, 19.20±0.2°, 20.59±0.2°, 22.88±0.2°, 23.07±0.2°, 24.32±0.2° and 25.84±0.2°.

In some embodiments, the hydrochloride of the present invention is hydrochloride crystal form B, the X-ray powder diffraction pattern of the hydrochloride crystal form B comprises diffraction peaks with 2θ angles of 6.38±0.2°, 10.23±0.2°, 11.37±0.2°, 12.73±0.2°, 13.14±0.2°, 16.13±0.2°, 16.45±0.2°, 17.10±0.2°, 17.43±0.2°, 18.06±0.2°, 18.28±0.2°, 19.20±0.2°, 20.04±0.2°, 20.59±0.2°, 21.43±0.2°, 22.21±0.2°, 22.39±0.2°, 22.88±0.2°, 23.07±0.2°, 23.56±0.2°, 23.80±0.2°, 24.32±0.2°, 25.84±0.2°, 26.47±0.2°, 26.97±0.2°, 27.61±0.2°, 28.25±0.2°, 28.80±0.2°, 29.41±0.2°, 30.58±0.2°, 31.11±0.2°, 31.59±0.2°, 32.10±0.2°, 32.77±0.2°, 33.28±0.2°, 33.67±0.2°, 34.75±0.2°, 35.21±0.2°, 36.12±0.2°, 36.55±0.2°, 37.28±0.2°, 38.13±0.2°, 38.64±0.2° and 38.97±0.2°.

In some embodiments, the hydrochloride of the present invention is hydrochloride crystal form B, and the hydrochloride crystal form B has an X-ray powder diffraction pattern substantially as shown in FIG. 23.

In some embodiments, the hydrochloride of the present invention is hydrochloride crystal form B, and the differential scanning calorimetry pattern of the hydrochloride crystal form B comprises an endothermic peak of 220.76° C.±3° C.

In some embodiments, the hydrochloride of the present invention is hydrochloride crystal form B, and the hydrochloride crystal form B has a differential scanning calorimetry pattern substantially as shown in FIG. 24.

In some embodiments, the phosphate of the present invention is phosphate crystal form C, the X-ray powder diffraction pattern of the phosphate crystal form C comprises diffraction peaks with 2θ angles of 5.44±0.2°, 6.11±0.2°, 14.67±0.2°, 15.83±0.2°, 17.35±0.2° and 19.22±0.2°.

In some embodiments, the phosphate of the present invention is phosphate crystal form C, the X-ray powder diffraction pattern of the phosphate crystal form C comprises diffraction peaks with 2θ angles of 5.44±0.2°, 6.11±0.2°, 11.30±0.2°, 12.23±0.2°, 13.82±0.2°, 14.67±0.2°, 15.83±0.2°, 17.35±0.2°, 19.22±0.2° and 25.30±0.2°.

In some embodiments, the phosphate of the present invention is phosphate crystal form C, the X-ray powder diffraction pattern of the phosphate crystal form C comprises diffraction peaks with 2θ angles of 3.91±0.2°, 5.44±0.2°, 6.11±0.2°, 11.30±0.2°, 12.23±0.2°, 13.82±0.2°, 14.67±0.2°, 15.10±0.2°, 15.83±0.2°, 16.49±0.2°, 17.00±0.2°, 17.35±0.2°, 18.47±0.2°, 18.68±0.2°, 19.22±0.2°, 20.00±0.2°, 20.49±0.2°, 20.87±0.2°, 21.21±0.2°, 21.43±0.2°, 22.15±0.2°, 22.67±0.2°, 23.29±0.2°, 24.34±0.2°, 24.70±0.2°, 25.05±0.2°, 25.30±0.2°, 25.88±0.2°, 26.37±0.2°, 26.76±0.2°, 27.44±0.2°, 28.02±0.2°, 30.06±0.2°, 30.86±0.2°, 32.97±0.2°, 35.19±0.2°, 35.82±0.2°, 37.31±0.2°, 39.38±0.2°, 41.99±0.2°, 45.36±0.2° and 47.13±0.2°.

In some embodiments, the phosphate of the present invention is phosphate crystal form C, and the phosphate crystal form C has an X-ray powder diffraction pattern substantially as shown in FIG. 25.

In some embodiments, the phosphate of the present invention is phosphate crystal form C, and the differential scanning calorimetry pattern of the phosphate crystal form C comprises an endothermic peak of 172.9° C.±3° C.

In some embodiments, the phosphate of the present invention is phosphate crystal form C, and the phosphate crystal form C has a differential scanning calorimetry pattern substantially as shown in FIG. 26.

In other aspect, provided herein is a N,N-dimethylformamide complex of a compound having Formula (I) or (Ia),

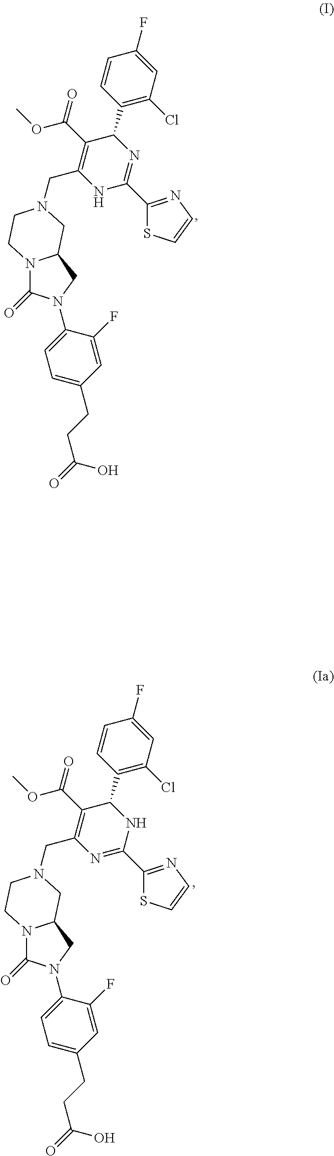 [SMILES: COC(=O)C1=C(CN2CCN3C(=O)N(c4ccc(CCC(=O)O)cc4F)C[C@@H]3C2)NC(c2nccs2)=N[C@H]1c1ccc(F)cc1Cl ; COC(=O)C1=C(CN2CCN3C(=O)N(c4ccc(CCC(=O)O)cc4F)C[C@@H]3C2)N=C(c2nccs2)N[C@H]1c1ccc(F)cc1Cl]

wherein the X-ray powder diffraction pattern of the N,N-dimethylformamide complex comprises diffraction peaks with 2θ angles of 10.31±0.2°, 10.91±0.2°, 17.04±0.2°, 19.18±0.2°, 20.17±0.2°, 21.83±0.2° and 24.41±0.2°.

In some embodiments, the X-ray powder diffraction pattern of the N,N-dimethylformamide complex comprises diffraction peaks with 2θ angles of 6.30±0.2°, 10.31±0.2°, 10.91±0.2°, 14.89±0.2°, 16.54±0.2°, 17.04±0.2°, 19.18±0.2°, 20.17±0.2°, 21.83±0.2° and 24.41±0.2°.

In some embodiments, the X-ray powder diffraction pattern of the N,N-dimethylformamide complex comprises diffraction peaks with 2θ angles of 6.30±0.2°, 7.19±0.2°, 8.85±0.2°, 10.31±0.2°, 10.91±0.2°, 11.36±0.2°, 11.93±0.2°, 12.53±0.2°, 12.93±0.2°, 13.93±0.2°, 14.89±0.2°, 15.31±0.2°, 15.90±0.2°, 16.54±0.2°, 17.04±0.2°, 17.94±0.2°, 18.39±0.2°, 18.69±0.2°, 19.18±0.2°, 20.17±0.2°, 20.70±0.2°, 20.96±0.2°, 21.60±0.2°, 21.83±0.2°, 22.18±0.2°, 22.49±0.2°, 22.74±0.2°, 23.37±0.2°, 23.77±0.2°, 24.41±0.2°, 24.70±0.2°, 25.13±0.2°, 25.71±0.2°, 26.14±0.2°, 26.45±0.2°, 27.44±0.2°, 28.02±0.2°, 28.30±0.2°, 28.76±0.2°, 29.52±0.2°, 30.12±0.2°, 30.68±0.2°, 31.18±0.2°, 31.66±0.2°, 31.98±0.2°, 33.24±0.2°, 33.82±0.2°, 34.44±0.2°, 34.76±0.2°, 36.00±0.2°, 37.34±0.2°, 37.83±0.2°, 38.92±0.2° and 39.61±0.2°.

In some embodiments, the N,N-dimethylformamide complex of the present invention has an X-ray powder diffraction pattern substantially as shown in FIG. 7.

In some embodiments, the differential scanning calorimetry pattern of the N,N-dimethylformamide complex of the present invention comprises an endothermic peak of 120.20° C.±3° C.

In some embodiments, N,N-dimethylformamide complex of the present invention has a differential scanning calorimetry pattern substantially as shown in FIG. 8.

In one aspect, provided herein is a pharmaceutical composition comprising the salt of the compound having Formula (I) or (Ia), complex or combination thereof, and a pharmaceutically acceptable carrier, excipient, diluent, adjuvant, vehicle or a combination thereof.

In other aspect, provided herein is use of the salt of the compound having Formula (I) or (Ia), the complex or pharmaceutical composition in the manufacture of a medicament for preventing, managing, treating or lessening viral disease in a patient. The use comprises administering to the patient a therapeutically effective amount of the crystal form or the pharmaceutical composition disclosed herein.

In some embodiments, the viral disease is a hepatitis B virus infection or a disease caused by hepatitis B virus infection.

In other embodiments, the disease caused by hepatitis B virus infection is liver cirrhosis or hepatocellular carcinoma.

In another aspect, provided herein is a method of the preventing, managing, treating or lessening viral disease in a patient comprising administering to the patient a therapeutically effective amount of the salt of the compound having Formula (I) or (Ia), the complex or pharmaceutical composition.

In some embodiments, the viral disease is a hepatitis B virus infection or a disease caused by hepatitis B virus infection.

In other embodiments, the disease caused by hepatitis B virus infection is liver cirrhosis or hepatocellular carcinoma.

In another aspect, provided herein is the salt of the compound having Formula (I) or (Ia), the complex or pharmaceutical composition disclosed herein for use in preventing, managing, treating or lessening viral disease in a subject.

In some embodiments, the viral disease is a hepatitis B virus infection or a disease caused by hepatitis B virus infection.

In other embodiments, the disease caused by hepatitis B virus infection is liver cirrhosis or hepatocellular carcinoma.

### DETAILED DESCRIPTION OF THE INVENTION

The invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described herein. In the event that one or more of the incorporated literature, patents, and similar materials differs from or contradicts this application, including but not limited to defined terms, term usage, described techniques, or the like, this application controls.

In the present invention, the crystal form of the salt of the compound having Formula (I) or (Ia) or the complex may comprise a solvent. In some cases, the solvent helps to improve the internal stability of the crystalline form of the salt of the compound having Formula (I) or (Ia) or the complex. The common solvent includes water, ethanol, methanol, isopropanol, acetone, isopropyl ether, diethyl ether, isopropyl acetate, n-heptane, tetrahydrofuran, dichloromethane, ethyl acetate, etc. As long as the above-mentioned crystal form with a certain amount of water or other solvents has any feature of the crystal form of the salt of the compound having Formula (I) or (Ia) or the complex, it should be considered to be included in the scope of the present invention.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Unless otherwise indicated, all technical and scientific terms used in the present invention have the same meaning as commonly understood by one of ordinary skilled in the art to which this invention pertains. All patents and publications referred to herein are incorporated by reference in their entirety. Although any methods and materials similar or identical to those described herein may be used in the practice or testing of the invention, but the methods, apparatus and materials described in the invention are preferred.

### Definitions and General Terminology

The term “comprise” is an open expression, it means comprising the contents disclosed herein, but don't exclude other contents.

In the present invention, “room temperature” refers to the temperature from 10° C. to 40° C. In some embodiments, “room temperature” refers to a temperature from 20° C. to 30° C.; in other embodiments, “room temperature” refers to a temperature from 25° C. to 30° C.

The term “pharmaceutically acceptable” provided herein refers to a substance that is acceptable for pharmaceutical applications from a toxicological point of view and does not adversely interact with the active ingredient.

The term “polymorphic” or “polymorphism” provided herein is defined that the same chemical molecule have the possibility of at least two different crystal arrangements.

The terms “crystalline form”, “crystal form”, “polymorph”, “polymorphs”, “crystalmodification”, “crystalline modification” and “polymorphic form” are understood to be synonymous. The compound or the solid crystal form of the salt or complex thereof provided herein includes but not limited to, mono- or multi-component crystals, and/or polymorphic compounds of compounds, solvates, hydrates, clathrates, eutectic, salt, salt solvent, salt hydrate.

Well-known techniques can be used to detect, identify, classify and qualitative polymorphs, these techniques include but not limited to: differential scanning calorimetry (DSC), thermogravimetric analysis (TGA), X-ray powder diffraction (XRPD), X-ray single crystal diffraction method, vibrational spectroscopy, solution calorimetry, solid-state nuclear magnetic resonance (SSNMR), Fourier transform infrared spectroscopy (FT-IR spectrum), Raman spectrum method, thermal stage optics microscopy, scanning electron microscopy (SEM), electron crystallography, and quantitative analysis, particle size analysis (PSA), surface area analysis, solubility and dissolution rate. The skilled person should understand that the graphical representation of the data may undergo small changes (such as peak relative intensity and peak position). The reasons for this are, for example, changes in the response of the instrument and changes in sample concentration and purity, which are well known to the skilled person. Nevertheless, the skilled person can compare the graphic data in the figures herein with the graphic data generated for the unknown crystal form, and can confirm whether the two are the same crystal form.

Unless otherwise specified, when the invention refers to spectra or data in graphical form (for example, XRPD, infrared, Raman, and NMR spectra), the term “peak” refers to a peak or other special feature caused by non-background noise that can be recognized by a person of ordinary skill in the art. The term “effective peak” refers to a peak that is at least the median size (e.g., height) of other peaks in the spectrum or data, or at least 1.5, 2 or 2.5 times the median size of other peaks.

“XRPD” refers to X-ray powder diffraction.

X-ray powder diffraction (XRPD) can detect the changes in crystal form, crystallinity, crystal state and other informations, it is a common means for identifying crystal form. The XRPD pattern refers to the diffraction pattern observed experimentally or the parameters derived from it. The X-ray powder diffraction pattern is characterized by the peak position (abscissa) and peak intensity (ordinate). The peak position primarily depends on the structure of the crystal form and is relatively insensitive to the experimental details, and its relative peak intensity depends on many factors associated with sample preparation and instrument geometry. Thus, in some embodiments, the crystalline form of the present invention is characterized by an XRPD map having certain peak positions, which is substantially as shown in the XRPD diagram provided in the drawings of the present invention at the same time, the 2θ of the XRPD pattern can be measured with an experimental error. The measurement of 2θ of the XRPD pattern may be slightly different between the different instruments and the different samples. Therefore, the value of 2θ can not be regarded as absolute. According to the conditions of the instrument used in this experiment, the diffraction peak has an error tolerance of ±0.1°, ±0.2°, 0.3°, ±0.4°, or ±0.5°. In some embodiments, the diffraction peak has an error tolerance of ±0.2°.

The term “2θ value” or “2θ angle” refers to the peak position in degrees of an experimental device based on an X-ray powder diffraction experiment and is a common abscissa unit of a diffraction pattern. The experimental device requires that when the reflection is diffracted, the incident beam forms an angle θ with a certain crystal plane, then the reflected beam is recorded at an angle of 2θ. It should be understood that the specific 2θ value of a specific polymorph refers to the 2θ value (in degrees) measured under the X-ray powder diffraction experimental conditions described herein.

In the context of the present invention, the 2θ values in the X-ray powder diffraction pattern are in degrees (°).

“Relative strength” means the ratio of the intensity of the other peaks to the intensity of the first strong peak when the intensity of the first strong peak in all the diffraction peaks of the X-ray powder diffraction pattern (XRD) is 100%.

Differential scanning calorimetry (DSC) is a technique of measuring the change of energy difference between a sample and an inert reference (commonly used α-Al₂O₃) varied with temperature by continuously heating or cooling under program control. The high melting peak of the DSC curve depends on many factors associated with sample preparation and instrument geometry, while the peak position is relatively insensitive to experimental details. Thus, in some embodiments, the crystal form of the present invention is characterized by an DCS map having characteristic peak positions, which is substantially as shown in the DCS diagram provided in the drawings of the present invention. At the same time, the DCS thermogram may have experimental errors. The peak position and peak value of DCS thermogram may be slightly different between the different instruments and the different samples. Therefore, the peak position or the peak value of the DSC endothermic peak can not be regarded as absolute. According to the condition of the instrument used in this test, the melting peak has error tolerances of ±1° C., ±2° C., ±3° C., ±4° C., or ±5° C. In some embodiments, the melting peak has an error tolerance of ±3° C. Differential scanning calorimetry (DSC) can also be used to detect whether the crystalline form has a crystal transformation or mixed crystal phenomenon.

The solid of same chemical composition, in different thermodynamic conditions, often form the different crystal structure of homogeneous isomers, or called variants, this phenomenon is called homogeneous polycrystalline or homogeneous polyphase phenomenon. When the temperature and pressure conditions change, the variants will change between each other, this phenomenon is called crystal transformation. Due to the crystal transformation, crystal mechanics, electrical, magnetic and other properties will be a huge change. This transition process is observed on differential scanning calorimetry (DSC) charts when the temperature of the crystal transition is within the measurable range, characterized in that the DSC diagram has an exothermic peak reflecting this transition, and has two or more endothermic peaks which are the characteristic endothermic peaks of different crystal forms before and after the transformation.

Thermogravimetric analysis (TGA) is a technique for measuring the change in the mass of a substance with temperature under the control of a program. It is suitable for examining the process of the solvent loss or the samples sublimation and decomposition. It can be presumed that the crystal contains crystal water or crystallization solvent. The quality variety of the TGA curve shown depends on a number of factors, containing the sample preparation and the instrument. The quality change from the TGA test varies slightly between the different instruments and between the different samples. According to the condition of the instrument used in this test, there is a ±0.1% error tolerance for the mass change.

“Amorphous” or “amorphous form” refers to a substance formed when the mass point (molecule, atom, ion) of a substance is arranged in a non-periodic manner in a three-dimensional space, characterized by an X-ray powder diffraction pattern with diffuse undisturbed peaks. Amorphization is a special physical form of solid matter, its locally ordered structural features suggest that it is inextricably linked with the crystalline material. The amorphous form of the material can be obtained by a number of methods known in the field. Such methods include, but are not limited to, quenching, anti-solvent flocculation, ball milling, spray drying, freeze drying, wet granulation and solid dispersion techniques.

“Solvent” refers to a substance (typically a liquid) that is capable of completely or partially dissolving another substance (typically a solid). Solvents for use in the practice of this invention include, but are not limited to, water, acetic acid, diethyl ether, isopropyl ether, petroleum ether, isopropyl acetate, methyl tert-butyl ether, n-heptane, acetone, acetonitrile, benzene, chloroform, carbon tetrachloride, dichloromethane, dimethyl methylene sulfone, 1,4-dioxane, ethanol, ethyl acetate, n-butanol, tert-butanol, N,N-dimethylacetamide, N,N-dimethylformamide, formamide, formic acid, hexane, isopropanol, methanol, methyl ethyl ketone, 1-methyl-2-pyrrolidone, mesitylene, nitromethane, polyethylene glycol, n-propanol, 2-acetone, pyridine, tetrahydrofuran, toluene, xylene, mixtures thereof, and the like.

“Anti-solvent” refers to a fluid that promotes the precipitation of a product (or product precursor) from a solvent. The anti-solvent may comprise a cold gas, or a fluid that promotes the precipitation of the product by chemical reaction or reduces the solubility of the product in the solvent; it may be the same liquid as the solvent but at a different temperature, or it may be a liquid different from the solvent.

“Solvate” refers to a compound that having a solvent on a surface, in a lattice, or having a solvent on a surface and in a lattice. The solvent can be water, acetic acid, acetone, acetonitrile, benzene, chloroform, carbon tetrachloride, dichloromethane, dimethyl sulfoxide, 1,4-dioxane, ethanol, ethyl acetate, butanol, tert-butanol, N,N-dimethylacetamide, N,N-dimethylformamide, formamide, formic acid, heptane, hexane, isopropanol, methanol, methyl ethyl ketone, methyl pyrrolidone, mesitylene, nitromethane, polyethylene glycol, propanol, 2-acetone, pyridine, tetrahydrofuran, toluene, xylene, mixtures thereof, and the like. A specific example of the solvate is a hydrate in which the solvent on the surface, in the lattice or on the surface and in the lattice is water. On the surface, in the lattice or on the surface and in the lattice of the substance, the hydrate may or may not have any solvent other than water.

The term “equivalent” or its abbreviation “eq” provided herein refers to the equivalent amount of other raw materials required based on the basic raw materials used in each step (1 equivalent) in accordance with the equivalent relationship of the chemical reaction.

Crystal form or amorphous can be identified by a variety of technical means, such as X-ray powder diffraction (XRPD), infrared absorption spectroscopy (IR), melting point method, differential scanning calorimetry (DSC), thermogravimetric analysis (TGA), Nuclear magnetic resonance, Raman spectroscopy, X-ray single crystal diffraction, dissolution calorimetry, scanning electron microscopy (SEM), quantitative analysis, solubility and dissolution rate.

The term “substantially as shown in the figure” refers to at least 50%, or at least 60%, or at least 70%, or at least 80%, or at least 80%, or at least 90%, or at least 95%, or at least 99% of the peaks are shown in the X-ray powder diffraction pattern or DSC pattern or Raman spectra pattern or infrared spectra pattern.

The “peak” refers to a feature that a person skilled in the art can recognize without belonging to background noise when referring to a spectrum or/and data that appears in the figure.

In the context of the present invention, when used or whether or not used the word, such as “about”, it means that within a given value or range of 10% or less, appropriately within 5%, especially within 1%. Or, for those of ordinary skill in the art, the term “about” means within an acceptable standard error range of the mean value. When a number with an N value is made public, any number within N+/−1%, N+/−2%, N+/−3%, N+/−5%, N+/−7%, N+/−8%, or N+/−10% is opened clearly, wherein “+/−” means plus or minus.

Unless otherwise stated, structures depicted herein are also meant to include all isomeric (e.g., enantiomeric, diastereomeric, and geometric) forms of the structure; for example, the R and S configurations for each asymmetric center, (Z) and (E) double bond isomers, and (Z) and (E) conformational isomers. Therefore, single stereochemical isomers as well as enantiomeric, diastereomeric, or geometric mixtures of the present compounds are within the scope disclosed herein.

The term “tautomer” or “tautomeric form” provided herein refers to structural isomers of different energies which are interconvertible via a low energy barrier. Where tautomerization is possible (e.g. in solution), a chemical equilibrium of tautomers can be reached. For example, proton tautomers (also called prototropic tautomers) include interconversion through proton migration, such as 3-(4-((S)-7-(((R)-6-(2-chloro-4-fluorophenyl)-5-(methoxycarbonyl)-2-(thiazol-2-yl)-3,6-dihydropyrimidin-4-yl)methyl)-3-oxohexahydroimidazo[1,5-a]pyrazine-2(3H)-yl)-3-fluorophenyl)propionic acid and 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid are tautomers of each other. Valence tautomers include interconversions by reorganization of some of the bonding electrons. Unless otherwise stated, all tautomeric forms of the compounds disclosed herein are within the scope of the invention.

Stereochemical definitions and conventions used herein generally follow S. P. Parker Ed., McGraw-Hill Dictionary of Chemical Terms (1984) McGraw-Hill Book Company, New York and Eliel et al., “Stereochemistry of Organic Compounds”, John Wiley & Sons, Inc., New York, 1994. The compounds disclosed herein may contain asymmetric or chiral centers, and therefore exist in different stereoisomeric forms. It is intended that all stereoisomeric forms of the compounds disclosed herein, including, but not limited to, diastereomers, enantiomers and atropisomers, as well as mixtures thereof such as racemic mixtures, form part of the present invention. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process.

As used herein, “patient” refers to a human (including adults and children) or other animal. In some embodiments, “patient” refers to a human.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

A Pharmaceutical Composition Comprising a Salt of the Compound Having Formula (I) or (Ia) of the Present Invention, a Complex or a Combination Thereof

As described above, the pharmaceutical compositions disclosed herein further comprise a pharmaceutically acceptable excipient, which, as used herein, includes any and all solvents, solid excipients, diluents, binders, disintegrants, or other liquid excipients, dispersants, flavoring or suspending agents, surfactants, isotonic agents, thickeners, emulsifiers, preservatives, solid binder, glidants or lubricants and the like, as suited to the particular dosage form desired. As described in the following: In Remington: Troy et al., Remington: The Science and Practice of Pharmacy, 21st ed., 2005, Lippincott Williams & Wilkins, Philadelphia, and Swarbrick et al., Encyclopedia of Pharmaceutical Technology, eds. 1988-1999, Marcel Dekker, New York, both of which are herein incorporated by reference in their entireties, discloses various excipients used in formulating pharmaceutically acceptable compositions and known techniques for the preparation thereof. Except insofar as any conventional excipient incompatible with the compounds disclosed herein, such as by producing any undesirable biological effect or otherwise interacting in a deleterious manner with any other components of the pharmaceutically acceptable composition, its use is contemplated to be within the scope of this invention.

Some non-limiting examples of materials which can serve as pharmaceutically acceptable excipients include ion exchangers; aluminium; aluminum stearate; lecithin; serum proteins such as human serum albumin; buffer substances such as phosphates; glycine; sorbic acid; potassium sorbate; partial glyceride mixtures of saturated vegetable fatty acids; water; salts or electrolytes such as protamine sulfate, disodium hydrogen phosphate, potassium hydrogen phosphate, sodium chloride and zinc salts; colloidal silica; magnesium trisilicate; polyvinyl pyrrolidone; polyacrylates; waxes; polyethylene-polyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol and polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants.

The salt of the compound, the complex or pharmaceutical composition provided herein is suitable for treating acute and chronic viral infections of infectious hepatitis, especially it can effectively inhibit hepatitis B virus (HBV). It is suitable for treating or alleviating the virus-induced diseases of patients, especially the acute and chronic persistent HBV infection. Chronic viral diseases caused by HBV may cause the disease to become serious. Chronic hepatitis B virus infection can lead to liver cirrhosis and/or hepatocellular carcinoma in many cases.

The salt of the compound, the complex or pharmaceutical composition provided herein can be administered in any of the following modes: oral administration, spray inhalation, topical administration, rectal administration, nasal administration, vaginal administration, parenteral administration such as subcutaneous, intravenous, intramuscular, intraperitoneal, intrathecal, intraventricular, intrasternal, or intracranial injection or infusion, or medication with the aid of an explanted reservoir. The preferred modes of administration are oral administration, intramuscular injection, intraperitoneal administration or intravenous injection.

The salt of the compound, the complex or pharmaceutically acceptable composition provided herein can be administered in the form of a unit dosage. The dosage form can be a liquid dosage form or a solid dosage form. The liquid dosage form can be true solutions, colloids, microparticles, and suspensions. Other dosage forms such as tablets, capsules, dripping pills, aerosols, pills, powders, solutions, suspensions, emulsions, granules, suppositories, freeze-dried powder injections, etc.

Oral tablets and capsules may comprise excipients such as binders, e.g. syrup, gum arabic, sorbitol, tragacanth, or polyvinylpyrrolidone; fillers, e.g. lactose, sucrose, corn starch, calcium phosphate, sorbitol, glycine; lubricants, e.g. magnesium stearate, talc, polyethylene glycol, silica; disintegrating agents, e.g. potato starch; or acceptable moisturizers, e.g. sodium lauryl sulfate. The tablets can be coated by a method known in pharmaceutics.

Oral liquid can be made into a suspension, a solution, an emulsion, a syrup or an elixir of water and oil, or can be made into a dry product and supplements with water or other suitable media before use. This liquid formulation may comprise conventional additives, e.g. suspending agents, sorbitol, cellulose methyl ether, glucose syrup, gelatin, hydroxyethyl cellulose, carboxymethyl cellulose, aluminum stearate gel, hydrogenated food oils, emulsifiers, e.g. lecithin, sorbitan monooleate, gum arabic; or non-aqueous carriers (may comprise edible oils), e.g. almond oil, fats such as glycerin, ethylene glycol, or ethanol; preservatives, e.g. methyl or propyl p-hydroxybenzoate, sorbic acid. Flavoring or coloring agents can be added if necessary.

Suppositories may comprise conventional suppository bases, such as cocoa butter or other glycerides.

For parenteral administration, the liquid dosage form is usually made of the compound and a sterile carrier. The carrier is preferably water. Depending on the selected carrier and drug concentration, the compound can be dissolved in a carrier or made into a suspension solution. When making into an injection solution, the compound is dissolved in water first, then filtered and sterilized, and filled into a sealed bottle or ampoule.

When the compound is applied topically to the skin, it can be made into a form of appropriate ointment, lotion, or cream, wherein the active ingredient is suspended or dissolved in one or more carriers, and the carriers that can be used in the ointment formulation include, but are not limited to: mineral oil, liquid petrolatum, white petrolatum, propylene glycol, polyethylene oxide, polypropylene oxide, emulsifying wax and water; the carriers that can be used in the lotion and cream include, but are not limited to: mineral oil, sorbitan Stearate, Tween 60, cetyl ester wax, hexadecenyl alcohol, 2-octyldodecanol, benzyl alcohol and water.

Generally speaking, it has been proven to be advantageous that whether in human medicine or in veterinary medicine, the total amount of the active compound provided herein administered according to the invention is about 0.5 to 500 mg every 24 hours, preferably 1 to 100 mg/kg body weight. If appropriate, the active compound administered is divided into multiple single doses to achieve the desired effect. The amount of the active compound contained in a single dose is preferably from about 1 to 80 mg, more preferably from 1 to 50 mg/kg body weight, but may not be in accordance with the above-mentioned dosage, that is, depending on the type and weight of the subject, the nature and severity of the disease, type of formulation and mode of administration of the drug, as well as dosing cycle or time interval.

The pharmaceutical composition provided herein also contains an anti-HBV drug, wherein the anti-HBV drug is an HBV polymerase inhibitor, an immunomodulator or an interferon.

The HBV drug includes Lamivudine, Telbivudine, Tenofovir dipivoxil, Entecavir, Adefovir dipivoxil, Alfaferone, Alloferon, Simo interleukin, Claviudine, Emtricitabine, Faprovir, Interferon, Baoganling CP, Interferon, Interferon α-1b, Interferon α, Interferon α-2a, Interferon β-1a, Interferon α-2, Interleukin-2, Milvotate, Nitrazoxanide, Pegylated interferon α-2a, Ribavirin, Ruinterferon-A, Cizonan, Euforavac, Rintatolimod, Phosphazid, Heplisav, Interferon α-2b, Levamisole and Propakium, etc.

The Use of the Salt of the Compound Having Formula (I) or (Ia) of the Present Invention, the Complex or the Pharmaceutical Composition

On the other aspect, the present invention relates to a use of the salt, complex or the pharmaceutical composition provided herein in the manufacture of a medicament for preventing, managing, treating or lessening hepatitis B disease in patients, including administering a pharmaceutically acceptable effective amount to a patient. Hepatitis B disease refers to liver diseases caused by hepatitis B virus infection or hepatitis B virus infection, including acute hepatitis, chronic hepatitis, cirrhosis and hepatocellular carcinoma. Acute hepatitis B virus infection can be asymptomatic or manifest as acute hepatitis symptoms. Patients with chronic viral infections have active diseases that can progress to cirrhosis and liver cancer.

An “effective amount” or “effective dose” of the salt, complex and/or pharmaceutically acceptable composition is an amount that is effective in treating or lessening the severity of one or more of the aforementioned disorders. The complex or pharmaceutically acceptable composition is effective administered in a fairly wide dose range. For example, the daily dose is from about 0.1 mg to 1000 mg per kg, the compounds or pharmaceutically acceptable compositions can be administered in a single dose or in several divided doses a day. The complex and composition, according to the method disclosed herein, may be administered using any amount and any route of administration which is effective for treating or lessening the severity of the disorder or disease. The exact amount required will vary from subject to subject, depending on the species, age, and general condition of the subject, the severity of the infection, the particular agent, its mode of administration, and the like. The compound, salt, crystal form, complex or pharmaceutical composition can also be administered with one or more other therapeutic agents as discussed above.

### DESCRIPTION OF THE DRAWINGS

FIG. 1 is an X-ray powder diffraction (XRPD) pattern of the sulfate crystal form B of the compound having Formula (Ia).

FIG. 2 is a differential scanning calorimetry (DSC) pattern of the sulfate crystal form B of the compound having Formula (Ia).

FIG. 3 is an X-ray powder diffraction (XRPD) pattern of the L-arginine salt crystal form A of the compound having Formula (I).

FIG. 4 is a differential scanning calorimetry (DSC) pattern of the L-arginine salt crystal form A of the compound having Formula (I).

FIG. 5 is an X-ray powder diffraction (XRPD) pattern of the hydrochloride crystal form A of the compound having Formula (Ia).

FIG. 6 is a differential scanning calorimetry (DSC) pattern of the hydrochloride crystal form A of the compound having Formula (Ia).

FIG. 7 is an X-ray powder diffraction (XRPD) pattern of the N,N-dimethylformamide complex of the compound having Formula (I).

FIG. 8 is a differential scanning calorimetry (DSC) pattern of the N,N-dimethylformamide complex of the compound having Formula (I).

FIG. 9 is an X-ray powder diffraction (XRPD) pattern of the sulfate crystal form A of the compound having Formula (Ia).

FIG. 10 is a differential scanning calorimetry (DSC) pattern of the sulfate crystal form A of the compound having Formula (Ia).

FIG. 11 is an X-ray powder diffraction (XRPD) pattern of the phosphate crystal form A of the compound having Formula (Ia).

FIG. 12 is a differential scanning calorimetry (DSC) pattern of the phosphate crystal form A of the compound having Formula (Ia).

FIG. 13 is an X-ray powder diffraction (XRPD) pattern of the phosphate crystal form B of the compound having Formula (Ia).

FIG. 14 is a differential scanning calorimetry (DSC) pattern of the phosphate crystal form B of the compound having Formula (Ia).

FIG. 15 is an X-ray powder diffraction (XRPD) pattern of the methanesulfonate crystal form A of the compound having Formula (Ia).

FIG. 16 is a differential scanning calorimetry (DSC) pattern of the methanesulfonate crystal form A of the compound having Formula (Ia).

FIG. 17 is an X-ray powder diffraction (XRPD) pattern of the p-toluenesulfonate crystal form A of the compound having Formula (Ia).

FIG. 18 is a differential scanning calorimetry (DSC) pattern of the p-toluenesulfonate crystal form A of the compound having Formula (Ia).

FIG. 19 is an X-ray powder diffraction (XRPD) pattern of the benzenesulfonate crystal form A of the compound having Formula (Ia).

FIG. 20 is a differential scanning calorimetry (DSC) pattern of the benzenesulfonate crystal form A of the compound having Formula (Ia).

FIG. 21 is an X-ray powder diffraction (XRPD) pattern of the hydrobromide crystal form A of the compound having Formula (Ia).

FIG. 22 is a differential scanning calorimetry (DSC) pattern of the hydrobromide crystal form A of the compound having Formula (Ia).

FIG. 23 is an X-ray powder diffraction (XRPD) pattern of the hydrochloride crystal form B of the compound having Formula (Ia).

FIG. 24 is a differential scanning calorimetry (DSC) pattern of the hydrochloride crystal form B of the compound having Formula (Ia).

FIG. 25 is an X-ray powder diffraction (XRPD) pattern of the phosphate crystal form C of the compound having Formula (Ia).

FIG. 26 is a differential scanning calorimetry (DSC) pattern of the phosphate crystal form C of the compound having Formula (Ia).

FIG. 27 is an X-ray single crystal diffraction pattern of the hydrochloride of compound (Ia).

### EXAMPLES

Embodiments of the present invention are described in detail below, and examples of the embodiments are shown in Figures. The embodiments described below with reference to the Figures are exemplary and are intended to explain the present invention and should not be construed as limiting the present invention.

General Preparation and Testing Methods

The crystalline form can be prepared by a variety of methods, including but not limited to, for example, crystallization or recrystallization from a suitable solvent mixture; sublimation; solid state conversion from another phase; crystallization from a supercritical fluid; and spraying.

Techniques for the crystallization or recrystallization of the crystalline form of the solvent mixture include, but are not limited to, for example, solvent evaporation; reducing the temperature of the solvent mixture; crystal seeding of the supersaturated solvent mixture of the compound and/or its salt; freeze-drying of the solvent mixture; and adding anti-solvent to the solvent mixture. High-yield crystallization techniques can be used to prepare crystalline forms, including polymorphs.

The crystals of drugs (including polymorphs), preparation methods, and characterization of drug crystals are discussed in Solid-State Chemistry of Drugs, S. R. Byrn, R. R. Pfeiffer and J. G Stowell, Second Edition, SSCI, West Lafayette, Indiana (1999).

In which, in the crystallization technology using a solvent, the solvent is generally selected based on one or more factors, the factors include but are not limited to, for example, the solubility of the compound, the used crystallization technology, and the vapor pressure of the solvent. A combination of solvents may be used. For example, the compound may be solubilized in the first solvent to obtain a solution, and then an anti-solvent may be added to reduce the solubility of the compound in the solution and crystal formations precipitate. The anti-solvent is a solvent in which the compound has low solubility.

Seed crystal can be added to any crystallization mixture to promote crystallization.

Crystal seeding can be used to control the growth of specific polymorphs and/or to control the grain size distribution of the crystalline product. Therefore, the calculation of the amount of seed crystals required depends on the size of the available seed crystals and the expected size of the average product particles, such as describing in “Programmed Cooling Batch Crystallizers”, J W Mullin and J. Nyvlt, Chemical Engineering Science, 1971, 26, 369-377. Generally, small-sized seed crystals are required to effectively control the crystal growth in the batch. Small-sized seed crystals can be produced by sieving, grinding or micronizing of large crystals, or by microcrystallization of solutions. In the progress of grinding or micronizing of crystals, the changing of crystal form from the desired crystal form should be avoided (i.e., the crystal form becomes amorphous or other polymorphic forms).

The cooled crystallization mixture can be filtered under vacuum, and the separated solid product can be washed with a suitable solvent (for example, a cold recrystallization solvent). After washing, the product can be dried under nitrogen purge to obtain the desired crystal form. The product can be analyzed by suitable spectroscopic or analytical techniques, including but not limited to, for example, differential scanning calorimetry (DSC), X-ray powder diffraction (XRPD) and thermogravimetric analysis (TGA), to ensure that the crystalline form of the compound has been formed. The resulting crystalline form can be produced in an amount of greater than about 70% by weight separation yield based on the weight of the compound initially used in the crystallization process, preferably greater than about 90% by weight separation yield. The co-milling in the product can optionally be removed by co-grinding or sieving.

After reading the following detailed description, those of ordinary skill in the art can more easily understand the features and advantages of the present invention. It should be understood that, for reasons of clarity, some features of the invention described in the above and the context of the separate embodiments below may also be combined to form a single embodiment. On the contrary, for reasons of brevity, different features of the invention described in the context of a single embodiment may also be combined to form sub-combinations. The disclosure of the present invention is further illustrated by the following examples, but these examples should not be interpreted as the scope of the present invention or limited to the specific steps described therein.

In the examples described below, unless otherwise indicated, all temperatures are set forth in degrees Celsius (° C.). Reagents were purchased from commercial suppliers such as Aldrich Chemical Company, Arco Chemical Company and Alfa Chemical Company, and were used without further purification unless otherwise indicated. Common solvents were purchased from commercial suppliers such as Shantou XiLong Chemical Factory, Guangdong Guanghua Reagent Chemical Factory Co. Ltd., Guangzhou Reagent Chemical Factory, Tianjin YuYu Fine Chemical Ltd., Qingdao Tenglong Reagent Chemical Ltd., and Qingdao Ocean Chemical Factory.

The crystal form prepared by the present invention is identified according to the following method:

¹H NMR spectra were recorded by a Bruker Avance 400 MHz spectrometer or Bruker Avance III HD 600 spectrometer, using CDCl₃, DMSO-d₆, CD₃OD or d₆-acetone (reported in ppm) as solvent, and using TMS (0 ppm) or chloroform (7.26 ppm) as the reference standard. When peak multiplicities were reported, the following abbreviations were used: s (singlet), s, s (singlet, singlet), d (doublet), t (triplet), m (multiplet), br (broadened), dd (doublet of doublets), ddd (doublet of doublet of doublets), dt (doublet of triplets), ddt (doublet of doublet of triplets), td (triplet of doublets), brs (broadened singlet). Coupling constants J, when given, were reported in Hertz (Hz).

The X-ray powder diffraction (XRPD) analysis method used in the invention was: Empyrean diffractometer, the radiation source was (Cu, kα, Kα1 (Å): 1.540598; Kα2 (Å): 1.544426; Kα2/Kα1 intensity ratio: 0.50). Wherein the voltage was set at 45 KV, and the current was set at 40 mA. The powdery sample was prepared as a thin layer on a monocrystalline silicon sample rack and placed on a rotating sample stage, analyzed at a rate of 0.0167 steps in the range of 3°˜40°. Data Collector software was used to collect data, HighScore Plus software was used to process data, and Data Viewer software was used to read data.

Single crystal x-ray diffraction analysis method: Data were collected on an Agilent Technologies Gemini A Ultra serial diffractometer using Cu Kα radiation (λ=1.5418 Å). Indexing and processing of the measured intensity data were carried out with CrysAlis PRO procedure. The structure was solved by direct methods using SHELX-97 (Sheldrick, G M. SHELXTL-97, Program for Crystal Structure Solution and Refinement; University of Gottingen: Gottingen, Germany, 1997). The derived atomic parameters (coordinates and temperature factors) were refined through full matrix least-squares. The function minimized in the refinements was Σ_{w}(|Fₒ|−|F_{c}|)². R is defined as Σ∥Fₒ|−|F_{c}∥/Σ|Fₒ|, while R_{w}=[Σ_{w}(|Fₒ|−|F_{c}|)²/Σ_{w}|Fₒ|₂]^{1/2} where w is an appropriate weighting function based on errors in the observed intensities. Difference maps were examined at all stages of refinement. The positions of hydrogens on nitrogen and oxygen were located in Fourier difference electron density maps. All the other hydrogen atoms were placed in calculated positions with fixed isotropic thermal parameters and included in the structure factor calculations in the final stage of full-matrix least-squares refinement. Simulated powder X-ray patterns were generated using Mercury procedure. Single crystal was selected by measuring 0.4×0.38×0.23 mm Single Crystal by single crystal diffraction analysis. The selected crystal was affixed to a thin glass fiber with a small amount of a light baseline, and mounted on a Gemini A Ultra single crystal diffractometer (Agilent Technologies).

The differential scanning calorimetry (DSC) analysis method used in the present invention was: performing a differential scanning calorimetry analysis using a TA Q2000 module with a thermal analysis controller. Data were collected and analyzed using TA Instruments Thermal Solutions software. Approximately 1-5 mg of the sample was accurately weighed into a specially crafted aluminum crucible with a lid and analyzed from room temperature to about 300° C. using a linear heating device at 10° C./min. During use, the DSC cell was purged with dry N₂ at 50 mL/min. The endothermic peak was drawn downward, and the data was analyzed and displayed by TA Universal Analysis.

The thermal gravimetric analysis (TGA) method used in the present invention was: performing a thermogravimetric analysis using a TA Q500 module with a thermal analysis controller. Data were collected and analyzed using TA Instruments Thermal Solutions software. Approximately 10 mg of the sample was accurately weighed into a platinum sample pan, and the sample was analyzed from room temperature to about 300° C. using a linear heating device at 10° C./min. During use, the TGA furnace chamber was purged with dry N₂.

The solubility of the present invention was determined using an Agilent 1200 High Performance Liquid Chromatograph VWD detector, and the chromatographic column model was Waters Xbridge-C18 (4.6×150 mm, 5 m). Detection wavelength was 250 nm, flow rate was 1.0 mL/min, the column temperature was 35° C., mobile phase was acetonitrile-water (v/v=40/60).

Low-resolution mass spectral (MS) data were also determined on an Agilent 6320 series LC-MS spectrometer equipped with G1312A binary pumps, a G1316A TCC (Temperature Control of Column, maintained at 30° C.), a G1329A autosampler and a G1315B DAD detector were used in the analysis. An ESI source was used on the LC-MS spectrometer.

Both LC-MS spectrometers were equipped with an Agilent Zorbax SB-C18, 2.1×30 mm, 5 m column. Injection volume was decided by the sample concentration. The flow rate was 0.6 mL/min. The HPLC peaks were recorded by UV-Vis wavelength at 210 nm and 254 nm. The mobile phase was 0.1% formic acid in acetonitrile (phase A) and 0.1% formic acid in ultrapure water (phase B). The gradient elution conditions were showed in Table 1:

TABLE 1
The gradient condition of the mobile phase
in Low-resolution mass spectrum analysis

| Time | A (CH₃CN, | B (H₂O, |
|---|---|---|
| (min) | 0.1% HCOOH) | 0.1% HCOOH) |
| 0~3 | 5~100 | 95~0 |
| 3~6 | 100 | 0 |
| 6~6.1 | 100~5 | 0~95 |
| 6.1~8 | 5 | 95 |

Purities of compounds were assessed by Agilent 1100 Series high performance liquid chromatography (HPLC) with UV detection at 210 nm and 254 nm (Zorbax SB-C18, 2.1×30 mm, 4 μm, 10 min, 0.6 mL/min flow rate, 5 to 95% (0.1% formic acid in CH₃CN) in (0.1% formic acid in H₂O). Column was operated at 40° C.

The following examples disclosed herein are presented to further describe the invention. However, these examples should not be used to limit the scope of the invention.

### 1. Preparation and Identification Examples

First, 3-(4-((S)-7-(((R)-6-(2-chloro-4-fluorophenyl)-5-(methoxycarbonyl)-2-(thiazol-2-yl)-3,6-dihydropyrimidin-4-yl)methyl)-3-oxohexahydroimidazo[1,5-a]pyrazine-2(3H)-yl)-3-fluorophenyl)propionic acid (i.e., the compound having Formula (I)) was obtained according to the preparation method described in Example 3 of the application WO2019076310.

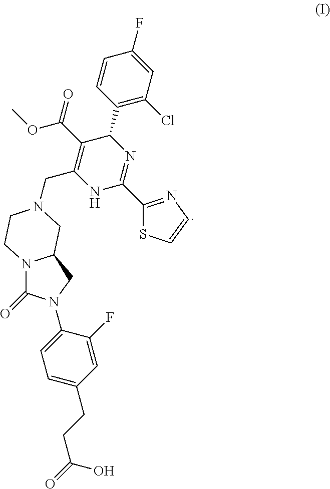 [SMILES: COC(=O)C1=C(CN2CCN3C(=O)N(c4ccc(CCC(=O)O)cc4F)C[C@@H]3C2)NC(c2nccs2)=N[C@H]1c1ccc(F)cc1Cl]

### Example 1

### 3-(4-((S)-7-(((R)-6-(2-chloro-4-fluorophenyl)-5-(methoxycarbonyl)-2-(thiazol-2-yl)-1,6-dihydro pyrimidin-4-yl)methyl)-3-oxohexahydroimidazo[1,5-a]pyrazine-2(3H)-yl)-3-fluorophenyl)prop ionic acid sulfate crystal form B

Preparation:

3-(4-((S)-7-(((R)-6-(2-chloro-4-fluorophenyl)-5-(methoxycarbonyl)-2-(thiazol-2-yl)-3, 6-dihydropyrimidin-4-yl)methyl)-3-oxohexahydroimidazo[1,5-a]pyrazine-2(3H)-yl)-3-fluorophenyl)propionic acid (1.00 g, 1.49 mmo), acetone (9 mL) and water (0.5 mL) were added into a dry reaction flask. Concentrated sulfuric acid (164 mg, 1.64 mmol) was diluted with acetone (1 mL) and the mixture was added to the flask. The resulting mixed solution was stirred for about 18 h at room temperature, then filtered with suction. The filter cake was washed with acetone (10 mL) and dried under vacuum at 60° C. for 12 h to obtain a yellow solid (1.00 g, 87.2%).

Results Identification:

(1) Ion Chromatography

The salt formation ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimiidazo[1,5-a]pyrazine-2(3H)-yl)-3-fluorophenyl)propionic acid sulfate crystal form B with sulfuric acid was determined by ion chromatography (T-00375). The method parameters were shown in the table below.

| Instrument model | | TI-00375 | | |
|---|---|---|---|---|
| Method | Eluent | 4.5 mM Na₂CO₃ + 0.8 mM NaHCO₃ | | |
| parameters | Flow rate | 1.0 mL/min | Column | 30° C. |
| | | | temperature | |
| | Column | IonPac AS23, DIONEX | Specification | 4 × 250 mm, Serial |
| | | | | No. 180522257 |
| | Quantitative | 100 μL | Detector | Suppressive conductivity |
| | loop | | | detector |

| | | |
|---|---|---|
| Sample | Control | 5 mL of SO₄²⁻ stock solution (10 μg/mL) was transferred to a 50 mL |
| test | solution | volumetric flask, ultrapure water was added to complete the volume, |
| | preparation | then shaken well. |
| | Sample | 1. The test product was precisely weighed about 40 mg and added into a |
| | solution | 50 mL volumetric flask, then 4 mL of methanol was added to the flask to |
| | preparation | dissolve the test product. 1 mL of AS23 stock solution was added into the |
| | | mixture, ultrapure water was added to complete the volume, then shaken |
| | | well; 2. 1 mL of the above solution was transferred into a 100 mL |
| | | volumetric flask, ultrapure water was added to complete the volume, |
| | | then shaken well and passed through the RP column. |

Notes:
AS23 stock solution was a mixed solution of 450 mM Na₂CO₃ + 80 mM NaHCO₃

The test results show that the salt formation molar ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid sulfate crystal form B with sulfuric acid is 1:1.

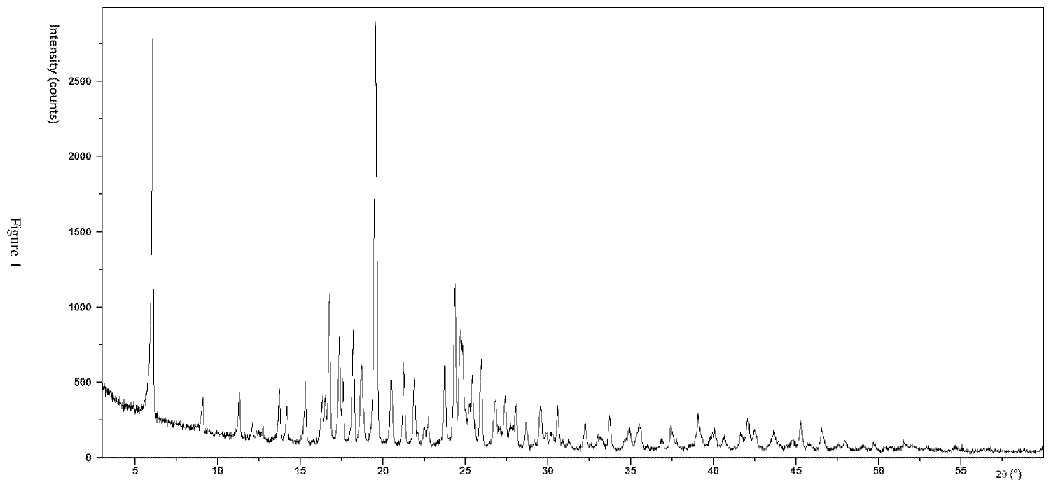

(2) Analysis and identification through Empyrean X-ray powder diffraction (XRPD): The obtained XRPD pattern is shown in FIG. 1. The X-ray powder diffraction pattern of the sulfate crystal form B contains the diffraction peaks with 2θ angles of 6.02°, 9.05°, 11.28°, 12.09°, 12.68°, 13.70°, 14.17°, 15.27°, 16.29°, 16.49°, 16.74°, 17.34°, 17.56°, 18.17°, 18.69°, 19.52°, 20.47°, 21.24°, 21.87°, 22.48°, 22.71°, 23.72°, 24.32°, 24.68°, 24.82°, 25.35°, 25.91°, 26.77°, 27.36°, 27.99°, 28.64°, 29.51°, 29.85°, 30.19°, 30.55°, 31.23°, 32.21°, 33.09°, 33.68°, 34.85°, 35.46°, 36.84°, 37.43°, 39.060 and 39.96°, and the diffraction peaks have an error tolerance of ±0.2°.

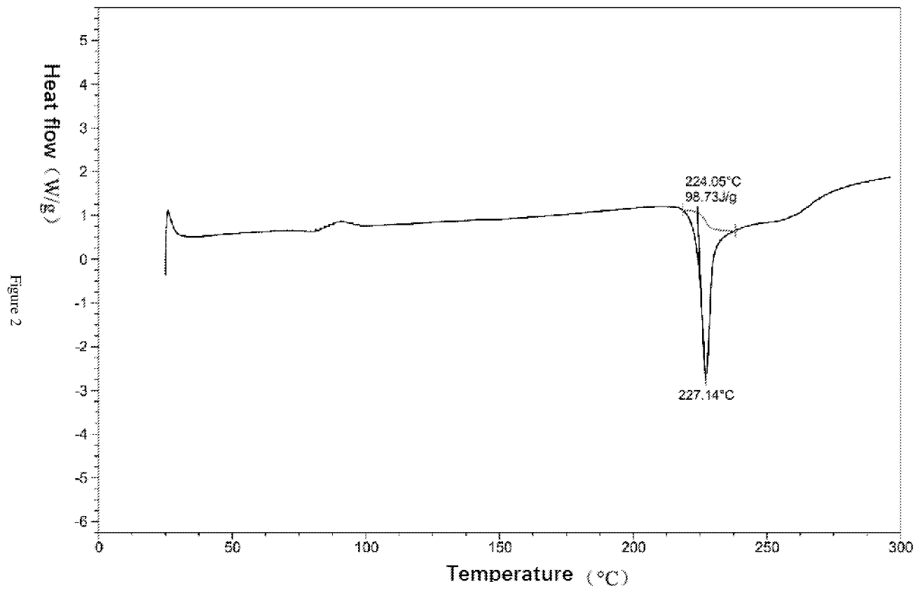

(3) Analysis and identification through TA Q2000 Differential Scanning Calorimetry (DSC): the scanning speed was 10° C./min, and the obtained DSC pattern was shown in FIG. 2, which contains an endothermic peak of 227.14° C. There is an error tolerance of ±3° C.

### Example 2

### 3-(4-((S)-7-(((R)-6-(2-chloro-4-fluorophenyl)-5-(methoxycarbonyl)-2-(thiazol-2-yl)-3,6-dihydropyrimidin-4-yl)methyl)-3-oxohexahydroimidazo[1,5-a]pyrazine-2(3H)-yl)-3-fluorophenyl)prop ionic acid L-arginine salt crystal form A

Preparation:

The compound having Formula (I) (100 g, 149 mmol) and methanol (1350 mL) were added in sequence into a reaction flask, then the mixture was stirred well and heated to 56° C. A solution of L-arginine (26.5 g, 149 mmol) in water (150 mL) was added dropwise to the flask. After the addition, the mixture was stirred for 20 min while keeping the temperature. Then the heating was stopped, the solution was cooled to room temperature, and continuously stirred for 12 h. The resulting solution was filtered, the filter cake was washed with methanol (300 mL) and dried at 60° C. for 24 h under vacuum to obtain a pale yellow solid (104.6 g, 83%).

Results Identification:

(1) NMR:¹H NMR (400 MHz, DMSO-d₆) δ 8.05 (d, J=3.1 Hz, 1H), 7.95 (d, J=3.1 Hz, 1H), 7.45-7.39 (m, 2H), 7.32 (t, J=8.3 Hz, 1H), 7.18 (td, J=8.5, 2.5 Hz, 1H), 7.11 (d, J=12.5 Hz, 1H), 7.03 (d, J=8.2 Hz, 1H), 6.05 (s, 1H), 3.98 (dd, J=40.1, 16.8 Hz, 2H), 3.87-3.74 (m, 3H), 3.53 (s, 3H), 3.40 (d, J=5.0 Hz, 1H), 3.25-3.21 (m, 1H), 3.13-3.00 (m, 3H), 2.91 (d, J=10.3 Hz, 2H), 2.76 (t, J=7.5 Hz, 2H), 2.38-2.15 (m, 4H), 1.79-1.69 (m, 1H), 1.62-1.51 (m, 3H).

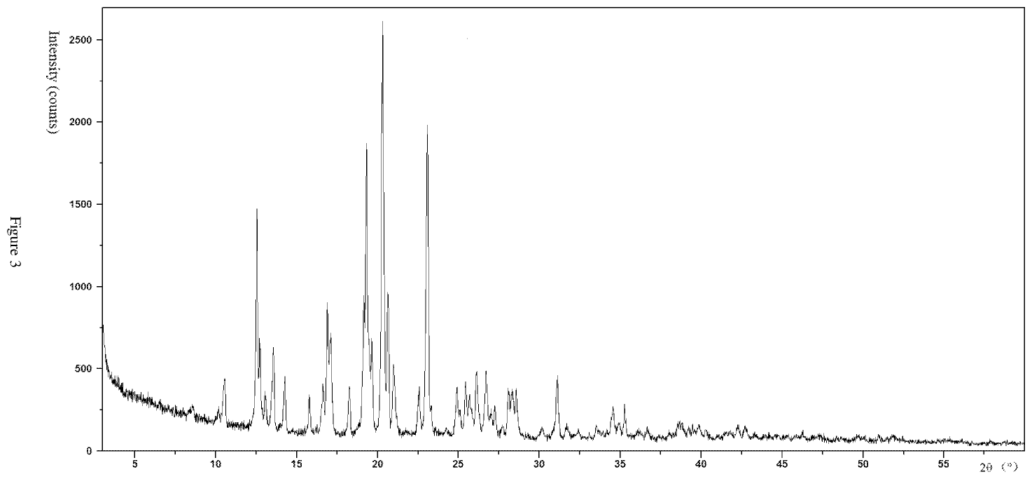

(2) Analysis and identification through Empyrean X-ray powder diffraction (XRPD): The obtained XRPD pattern is shown in FIG. 3. The X-ray powder diffraction pattern of the L-arginine salt crystal form A contains the diffraction peaks with 2θ angles of 8.50°, 10.50°, 12.52°, 12.71°, 13.05°, 13.52°, 14.23°, 15.76°, 16.60°, 16.88°, 17.07°, 18.22°, 19.11°, 19.30°, 19.58°, 20.29°, 20.61°, 20.98°, 22.53°, 23.04°, 24.90°, 25.41°, 25.68°, 26.11°, 26.68°, 27.22°, 28.07°, 28.29°, 28.54°, 30.12°, 31.06°, 31.68°, 33.55°, 34.50°, 34.89°, 35.24°, 36.12°, 36.65°, 38.680 and 39.80°, and the diffraction peaks have an error tolerance of ±0.2°.

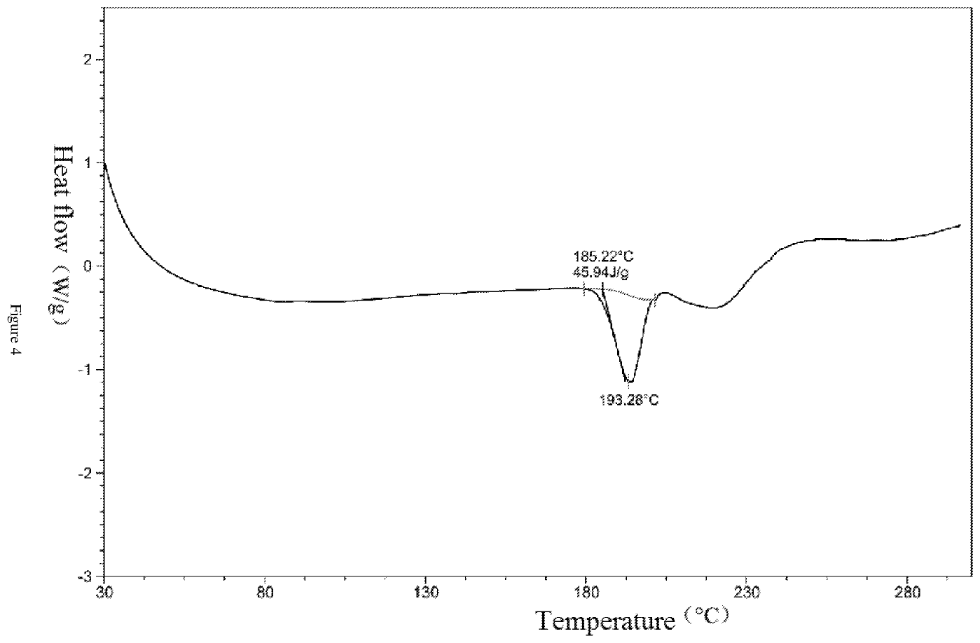

(3) Analysis and identification through TA Q2000 Differential Scanning Calorimetry (DSC): the scanning speed is 10° C./min, and the obtained DSC pattern is shown in FIG. 4, which contains an endothermic peak of 193.28° C. There is an error tolerance of ±3° C.

### Example 3

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)prop ionic acid hydrochloride crystal form A

Preparation:

The compound having Formula (I) (1.00 g, 1.49 mmol), acetone (9 mL) and water (0.2 mL) were added in sequence into a reaction flask, then the mixture was heated to 50° C. Concentrated hydrochloric acid (155 mg, 1.57 mmol, 37%) was diluted with acetone (1 mL), and the mixture was added to the flask. After the addition, the mixture was stirred for 20 min while keeping the temperature. Then the heating was stopped, the solution was cooled to room temperature. The mixture was continuously stirred at room temperature for 12 h. The resulting solution was filtered, the filter cake was washed with acetone (10 mL) and dried at 60° C. for 12 h under vacuum to obtain a yellow solid (879 mg, 83.4%).

Results Identification:

(1) Ion Chromatography

The salt formation ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid hydrochloride crystal form A with hydrochloric acid was determined by ion chromatography (TI-00375). The method parameters were shown in the table below.

| Instrument model | | TI-00375 | | |
|---|---|---|---|---|
| Method | Eluent | 4.5 mM Na₂CO₃ + 0.8 mM NaHCO₃ | | |
| parameters | Flow rate | 1.0 mL/min | Column | 30° C. |
| | | | temperature | |
| | Column | IonPac AS23, DIONEX | Specification | 4 × 250 mm, Serial |
| | | | | No. 180522257 |
| | Quantitative | 100 μL | Detector | Suppressive conductivity |
| | loop | | | detector |

| | | |
|---|---|---|
| Sample | Control | 5 mL of Cl⁻ stock solution (10 μg/mL) was transferred to a 50 mL |
| test | solution | volumetric flask. Ultrapure water was added to complete the volume, |
| | preparation | then shaken well. |
| | Sample | 1. The sample was precisely weighed about 25 mg and added into a 25 |
| | solution | mL volumetric flask, then 4 mL of methanol was added to the flask to |
| | preparation | dissolve the sample. 200 μL of 450 mM Na₂CO₃ solution was added |
| | | into the mixture, then the ultrapure water was added and shaken well. |
| | | The mixture was stood to clear and then made constant volume; 2. 1 mL |
| | | of the above solution was transferred into a 50 mL volumetric flask. |
| | | Ultrapure water was added to complete the volume, then shaken well |
| | | and passed through the RP column. |

The test results show that the salt formation molar ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid hydrochloride crystal form A with hydrochloric acid is 1:1.

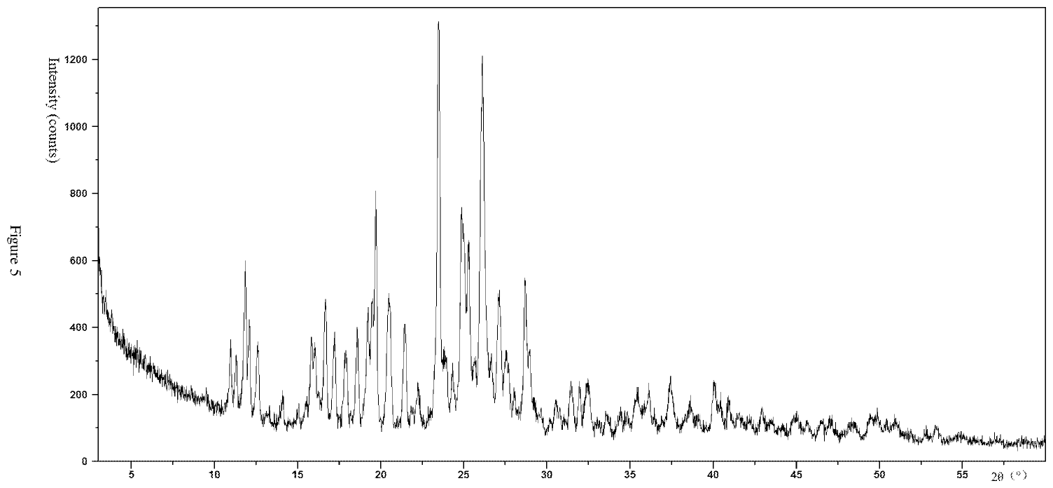

(2) Analysis and identification through Empyrean X-ray powder diffraction (XRPD): The obtained XRPD pattern is shown in FIG. 5. The X-ray powder diffraction pattern of the hydrochloride crystal form A contains the diffraction peaks with 2θ angles of 10.94°, 11.28°, 11.82°, 12.08°, 12.57°, 14.06°, 15.01°, 15.81°, 16.02°, 16.64°, 17.18°, 17.86°, 18.55°, 19.22°, 19.64°, 20.46°, 21.41°, 22.19°, 23.44°, 23.85°, 24.28°, 24.89°, 25.25°, 26.08°, 26.37°, 27.09°, 27.53°, 28.000, 28.650, 28.910, 30.530, 31.420, 31.920, 32.400, 33.580, 34.360, 35.380, 36.070, 37.390 and 38.58°, and the diffraction peaks have an error tolerance of ±0.2°.

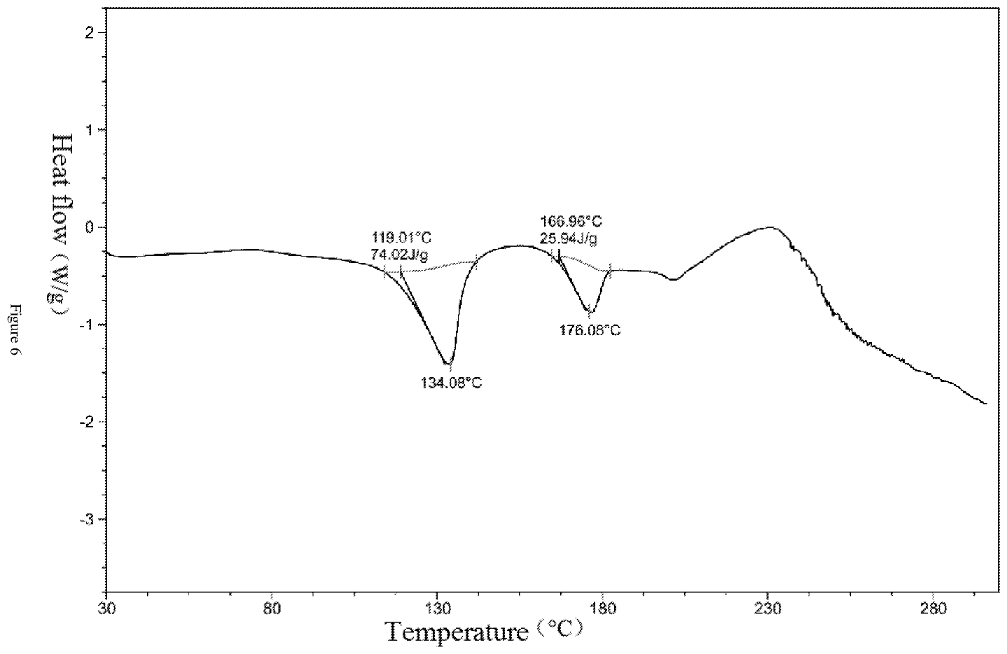

(3) Analysis and identification through TA Q2000 Differential Scanning Calorimetry (DSC): the scanning speed was 10° C./min, and the obtained DSC pattern was shown in FIG. 6, which contains endothermic peaks of 134.08° C. and 176.08° C. There is an error tolerance of ±3° C.

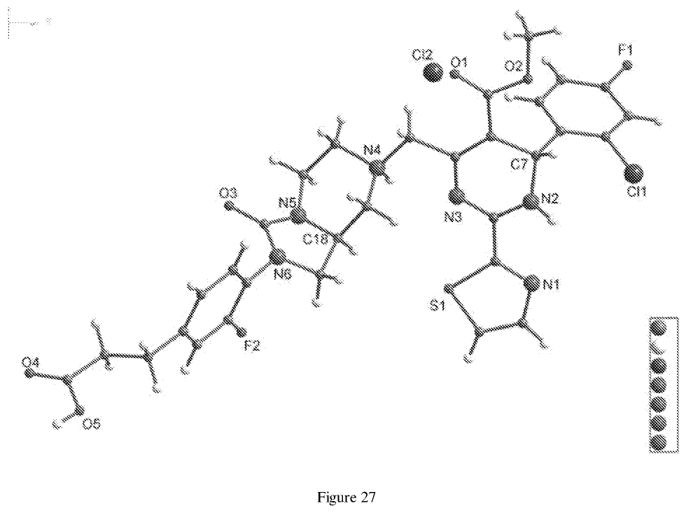

(4) Single crystal X-ray study: The structure contains 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid hydrochloride was proved as shown in FIG. 27.

### Example 4

### 3-(4-((S)-7-(((R)-6-(2-chloro-4-fluorophenyl)-5-(methoxycarbonyl)-2-(thiazol-2-yl)-3,6-dihydropyrimidin-4-yl)methyl)-3-oxohexahydroimidazo[1,5-a]pyrazine-2(3H)-yl)-3-fluorophenyl)prop ionic acid N,N-dimethylformamide complex

Preparation:

The compound having Formula (I) (5 g, 7.5 mmol) and ethyl acetate (35 mL) were added in sequence into a reaction flask, then the mixture was stirred at room temperature. After the solid was completely dissolved, DMF (1.6 g, 22 mmol) was added to the mixture and stirred at room temperature for 24 h. The resulting solution was filtered, the filter cake was washed with ethyl acetate (5 mL) and dried at 60° C. for 12 h under vacuum to obtain a pale yellow solid (4.34 g, 77.8%).

Results Identification:

(1) NMR: ¹H NMR (400 MHz, CH₃OH-d₄) δ 8.00 (s, 1H), 7.97 (d, J=3.1 Hz, 1H), 7.75 (d, J=3.1 Hz, 1H), 7.44 (dd, J=8.7, 6.1 Hz, 1H), 7.35 (t, J=8.3 Hz, 1H), 7.24 (dd, J=8.7, 2.6 Hz, 1H), 7.14-7.01 (m, 3H), 6.18 (s, 1H), 4.16 (d, J=16.9 Hz, 1H), 4.12-4.01 (m, 1H), 4.00-3.85 (m, 3H), 3.61 (s, 3H), 3.49 (dd, J=9.2, 4.5 Hz, 1H), 3.33-3.22 (m, 2H), 3.01 (s, 3H), 2.98-2.91 (m, 4H), 2.88 (s, 3H), 2.63 (t, J=7.5 Hz, 2H), 2.47 (td, J=11.7, 3.2 Hz, 1H), 2.36 (t, J=10.9 Hz, 1H).

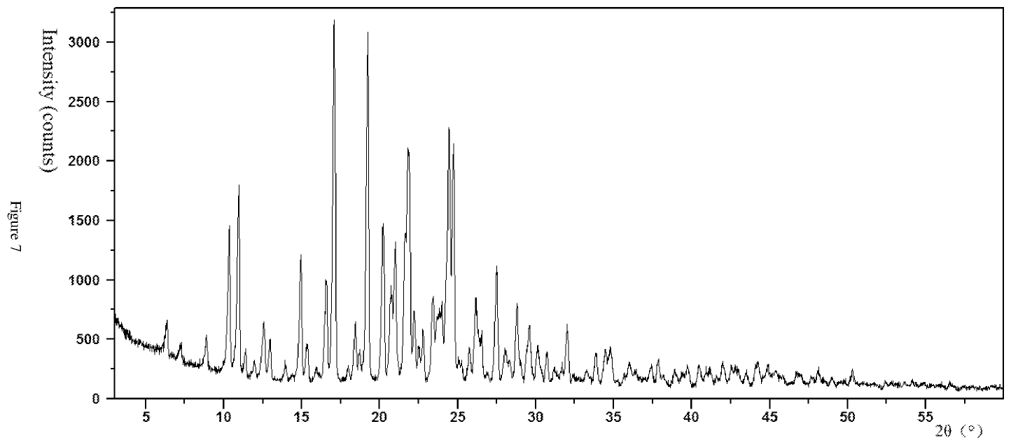

(2) Analysis and identification through Empyrean X-ray powder diffraction (XRPD): The obtained XRPD pattern is shown in FIG. 7. The X-ray powder diffraction pattern of the DMF complex of the compound having Formula (I) contains the diffraction peaks with 2θ angles of 6.30°, 7.19°, 8.85°, 10.31°, 10.91°, 11.36°, 11.93°, 12.53°, 12.93°, 13.93°, 14.89°, 15.31°, 15.90°, 16.54°, 17.04°, 17.94°, 18.39°, 18.69°, 19.18°, 20.17°, 20.70°, 20.96°, 21.60°, 21.83°, 22.18°, 22.49°, 22.74°, 23.37°, 23.77°, 24.41°, 24.70°, 25.13°, 25.71°, 26.14°, 26.45°, 27.44°, 28.02°, 28.30°, 28.76°, 29.52°, 30.12°, 30.68°, 31.18°, 31.66°, 31.98°, 33.24°, 33.82°, 34.44°, 34.76°, 36.00°, 37.34°, 37.83°, 38.920 and 39.61°, and the diffraction peaks have an error tolerance of ±0.2°.

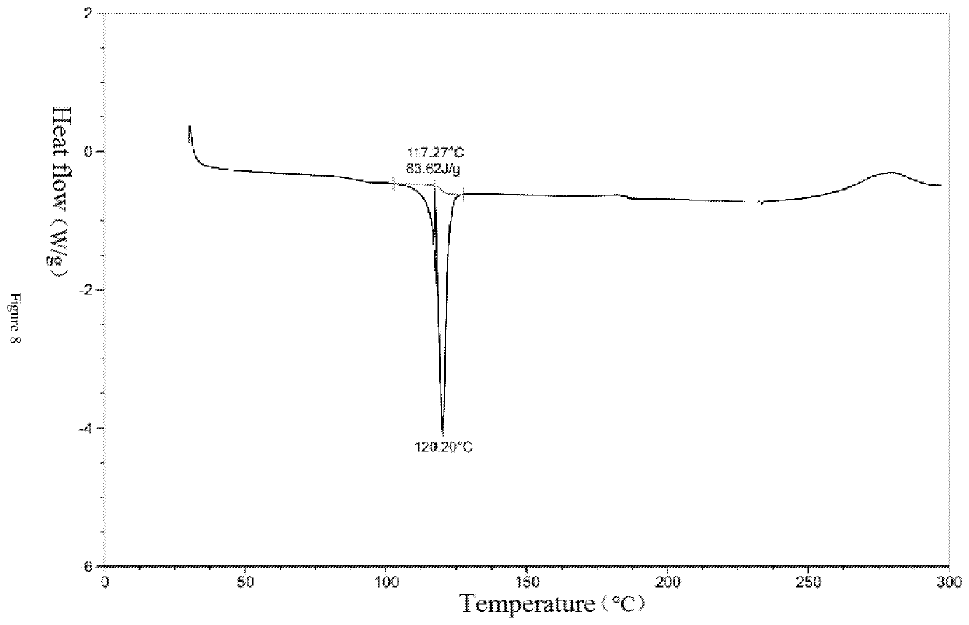

(3) Analysis and identification through TA Q2000 Differential Scanning Calorimetry (DSC): the scanning speed was 10° C./min, and the obtained DSC pattern was shown in FIG. 8, which contains an endothermic peak of 120.20° C. There is an error tolerance of ±3° C.

### Example 5

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)prop ionic acid sulfate crystal form A

Preparation:

The compound having Formula (I) (1.00 g, 1.49 mmol), acetone (9 mL), and water (0.1 mL) were added in sequence into a dry reaction flask. The mixture was heated to about 50° C. Concentrated sulfuric acid (165 mg, 1.65 mmol) was diluted with acetone (1 mL) and the mixture was added to the flask. After the addition, the mixture was continuously stirred for about 20 minutes and then the heating was stopped. The resulting mixture was stirred at room temperature for about 21 h and then filtered with suction. The filter cake was washed with acetone (10 mL) and dried at 60° C. for 12 h under vacuum to obtain a yellow solid (997 mg, 87.0%).

Results Identification:

(1) Ion chromatography: The salt formation ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid sulfate crystal form A with sulfuric acid was determined by ion chromatography (TI-00586). The method parameters were shown in the table below.

| Instrument model | | TI-00586 | | |
|---|---|---|---|---|
| Method | Eluent | 40 mM NaOH | | |
| parameters | Flow rate | 1.0 mL/min | Column | 30° C. |
| | | | temperature | |
| | Column | IonPac AS18, | Specification | 4 × 250 mm, Serial No.: 011532 |
| | | DIONEX | | |
| | Quantitative | 100 μL | Detector | Suppressive conductivity |
| | loop | | | detector |

| | | |
|---|---|---|
| Sample | Control | 5 mL of SO₄²⁻ stock solution (10 μg/mL) was transferred to a 50 mL |
| test | solution | volumetric flask, ultrapure water was added to complete the |
| | preparation | volume, then shaken well. |
| | | The sample was precisely weighed 25 mg and added into a 25 mL |
| | | volumetric flask, then 4 mL of methanol was added to the flask to |
| | Sample | dissolve the test product. 200 μL of 450 mM Na₂CO₃ solution was |
| | solution | added into the mixture, ultrapure water was added to complete the |
| | preparation | volume, then shaken well. The solution was taken through the 1.0 cc |
| | | RP pretreatment column, the first 3 mL of solution was discarded |
| | | and the additional filtrate was taken. |

The test results show that the salt formation molar ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid sulfate crystal form A with sulfuric acid is 1:1.

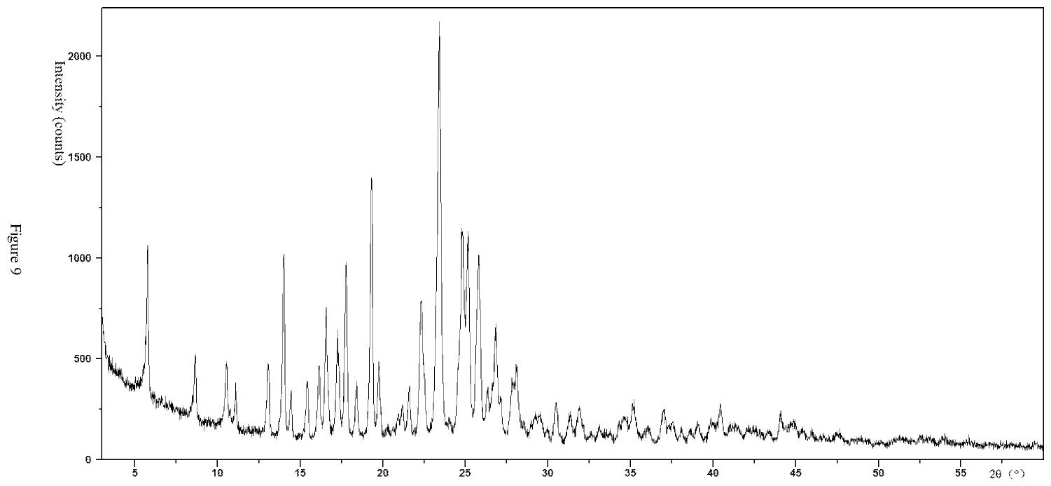

(2) Analysis and identification through Empyrean X-ray powder diffraction (XRPD): The obtained XRPD pattern is shown in FIG. 9. The X-ray powder diffraction pattern of the sulfate crystal form A contains the diffraction peaks with 2θ angles of 5.74°, 8.62°, 10.52°, 11.08°, 13.04°, 13.97°, 14.42°, 15.40°, 16.11°, 16.56°, 17.25°, 17.75°, 18.38°, 19.28°, 19.74°, 21.14°, 21.57°, 22.33°, 23.38°, 24.78°, 25.13°, 25.76°, 26.31°, 26.80°, 27.12°, 27.83°, 28.08°, 29.32°, 30.45°, 31.31°, 31.87°, 33.08°, 34.87°, 36.01°, 36.95°, 37.42°, 38.59°, 39.030 and 39.92°, and the diffraction peaks have an error tolerance of ±0.2°.

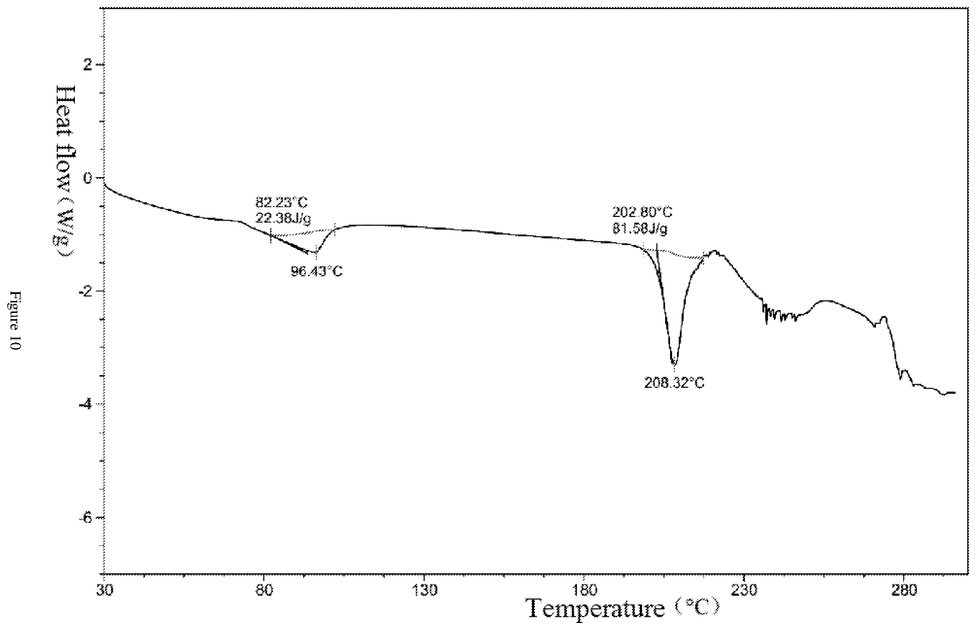

(3) Analysis and identification through TA Q2000 Differential Scanning Calorimetry (DSC): the scanning speed was 10° C./min, and the obtained DSC pattern was shown in FIG. 10, which contains endothermic peaks of 96.43° C. and 208.32° C. There is an error tolerance of ±3° C.

### Example 6

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)prop ionic acid phosphate crystal form A

Preparation:

The compound having Formula (I) (5 g, 7.45 mmol) and acetone (75 mL) were added in sequence into a dry reaction flask, the mixture was stirred and dissolved completely at room temperature and then heated to 50° C. A solution of phosphoric acid (2.6 g, 23 mmol, 85%) in water (1.5 mL) was added to the flask. After the addition, the mixture was stirred for 30 m while keeping the temperature. Then the heating was stopped, the solution was cooled to room temperature, and continuously stirred for 24 h. The resulting solution was filtered, the filter cake was washed with acetone (20 mL) and then dried at 60° C. for 12 h under vacuum to obtain a yellow solid (4.4 g, 68%).

Results Identification:

(1) Ion Chromatography

The salt formation ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid phosphate crystal form A with phosphoric acid was determined by ion chromatography (TI-00375). The method parameters were shown in the table below.

| Instrument model | | TI-00375 | | |
|---|---|---|---|---|
| Method | Eluent | 4.5 mM Na₂CO₃ + 0.8 mM NaHCO₃ | | |
| parameters | Flow rate | 1.0 mL/min | Column | 30° C. |
| | | | temperature | |
| | Column | IonPac AS23, | Specification | 4 × 250 mm, Serial |
| | | DIONEX | | No. 180522257 |
| | Quantitative | 100 μL | Detector | Suppressive conductivity |
| | loop | | | detector |

| | | |
|---|---|---|
| Sample | Control | 5 mL of PO₄³⁻ stock solution (10 μg/mL) was transferred to a 50 mL |
| test | solution | volumetric flask, ultrapure water was added to complete the volume, |
| | preparation | then shaken well. |
| | | The sample was precisely weighed about 22 mg and added into a 50 mL |
| | | volumetric flask, then 4 mL of methanol was added to the flask to |
| | Sample | dissolve the sample. 200 μL of 450 mM Na₂CO₃ solution was added |
| | solution | into the mixture, ultrapure water was added to complete the volume, |
| | preparation | then shaken well. The mixture was stood to clear and then made |
| | | constant volume. 1 mL of the above solution was transferred into a 100 |
| | | mL volumetric flask. Ultrapure water was added to complete the |
| | | volume, then shaken well. |

The test results show that the salt formation molar ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid phosphate crystal form A with phosphoric acid is 1:2.

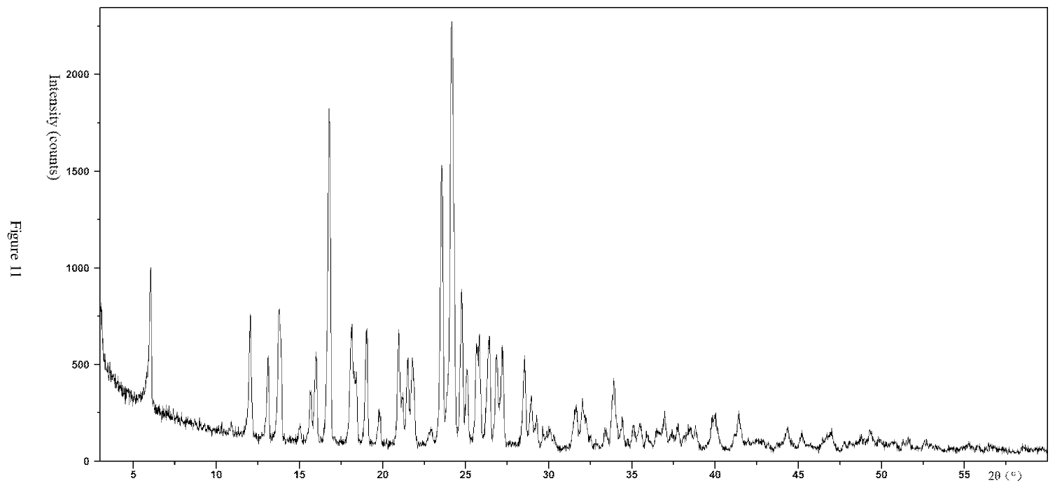

(2) Analysis and identification through Empyrean X-ray powder diffraction (XRPD): The obtained XRPD pattern is shown in FIG. 11. The X-ray powder diffraction pattern of the phosphate crystal form A contains the diffraction peaks with 2θ angles of 6.01°, 10.88°, 12.01°, 13.07°, 13.76°, 13.88°, 14.99°, 15.64°, 15.95°, 16.75°, 18.11°, 18.37°, 18.99°, 19.76°, 20.94°, 21.16°, 21.48°, 21.78°, 22.82°, 23.52°, 24.14°, 24.72°, 25.03°, 25.63°, 25.80°, 26.34°, 26.83°, 27.15°, 28.49°, 28.90°, 29.21°, 29.61°, 30.02°, 31.55°, 32.04°, 33.37°, 33.87°, 34.36°, 35.06°, 35.42°, 35.86°, 36.53°, 36.91°, 37.67°, 38.480 and 39.91°, and the diffraction peaks have an error tolerance of ±0.2°.

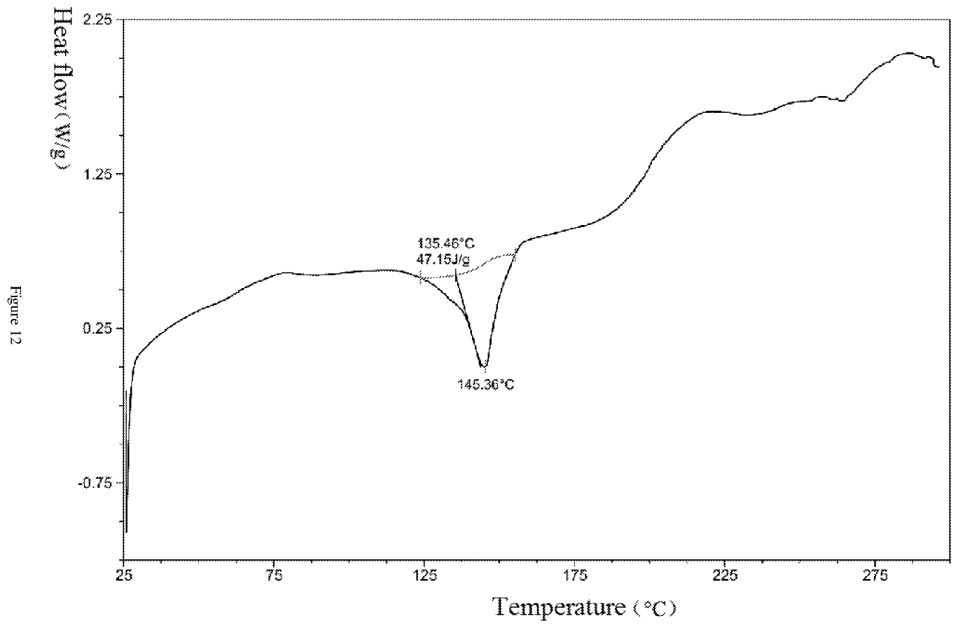

(3) Analysis and identification through TA Q2000 Differential Scanning Calorimetry (DSC): the scanning speed is 10° C./min, and the obtained DSC pattern is shown in FIG. 12, which contains an endothermic peak of 145.36° C. There is an error tolerance of ±3° C.

### Example 7

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluoro phenyl)propionic acid phosphate crystal form B

Preparation:

The compound having Formula (I) (1 g, 1.49 mmol) and acetone (10 mL) were added in sequence into a dry reaction flask. The mixture was stirred and dissolved completely at room temperature, then a solution of phosphoric acid (207 mg, 1.80 mmol, 85%) in acetone (5 mL) was added to the flask. After the addition, the mixture was stirred for 12 h at room temperature. The resulting solution was filtered, the filter cake was washed with acetone (6 mL) and then dried at 60° C. for 12 h under vacuum to obtain a yellow solid (0.6 g, 52%).

Results Identification:

(1) Ion Chromatography

The salt formation ratio of the compound having Formula (Ia) 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid phosphate crystal form B with phosphoric acid was determined by ion chromatography (TI-00375). The method parameters were shown in the table below.

| Instrument model | | TI-00375 | | |
|---|---|---|---|---|
| Method | Eluent | 4.5 mM Na₂CO₃ + 0.8 mM NaHCO₃ | | |
| parameters | Flow rate | 1.0 mL/min | Column | 30° C. |
| | | | temperature | |
| | Column | IonPac AS23, | Specification | 4 × 250 mm, Serial |
| | | DIONEX | | No. 180522257 |
| | Quantitative | 100 μL | Detector | Suppressive conductivity |
| | loop | | | detector |

| | | |
|---|---|---|
| Sample | Control | 5 mL of PO₄³⁻ stock solution (10 μg/mL) was transferred to a 50 mL |
| test | solution | volumetric flask, ultrapure water was added to complete the volume, |
| | preparation | then shaken well. |
| | | The sample was precisely weighed about 22 mg and added into a 50 mL |
| | | volumetric flask, then 4 mL of methanol was added to the flask to |
| | | dissolve the sample. 200 μL of 450 mM Na₂CO₃ solution was added |
| | | into the mixture, ultrapure water was added to complete the volume, |
| | Sample | then shaken well. The mixture was stood to clear and then made |
| | solution | constant volume. 1 mL of the above solution was transferred into a 100 |
| | preparation | mL volumetric flask. Ultrapure water was added to complete the |
| | | volume, then shaken well. |

The test results show that the salt formation molar ratio of the compound having Formula (Ia) 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophen yl)propionic acid phosphate crystal form B with phosphoric acid is 1:2.

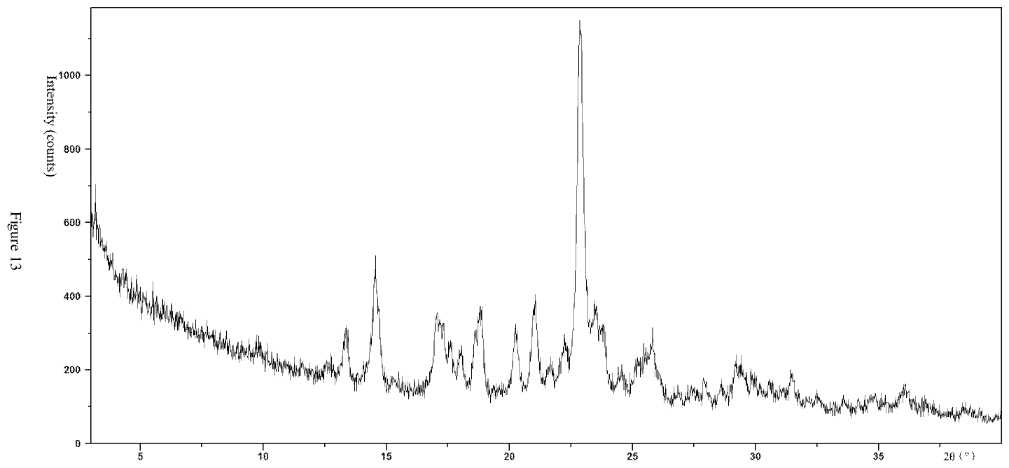

(2) Analysis and identification through Empyrean X-ray powder diffraction (XRPD): The obtained XRPD pattern is shown in FIG. 13. The X-ray powder diffraction pattern of the phosphate crystal form B contains the diffraction peaks with 2θ angles of 13.37°, 14.55°, 17.01°, 18.04°, 18.84°, 20.25°, 21.03°, 22.21°, 22.83°, 23.83°, 24.51°, 25.80°, 27.94°, 29.18°, 31.43°, 32.45° and 36.09°, and the diffraction peaks have an error tolerance of ±0.2°.

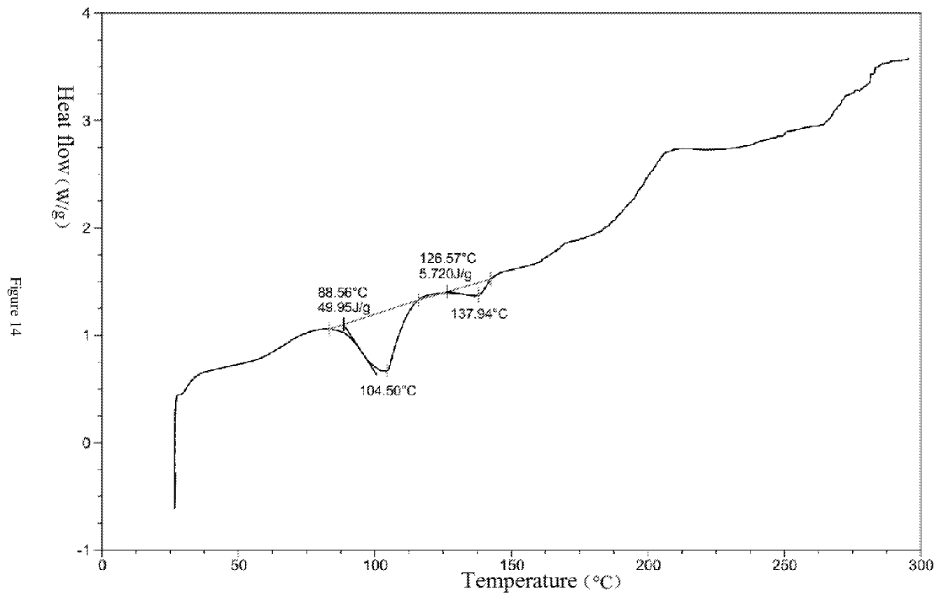

(3) Analysis and identification through TA Q2000 Differential Scanning Calorimetry (DSC): the scanning speed was 10° C./min, and the obtained DSC pattern was shown in FIG. 14, which contains endothermic peaks of 104.50° C. and 137.94° C. There is an error tolerance of ±3° C.

### Example 8

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluoro phenyl)propionic acid methanesulfonate crystal form A

Preparation:

The compound having Formula (I) (0.5 g, 0.75 mmol) and water-saturated ethyl acetate (5 mL) were added in sequence into a dry reaction flask, then the mixture was heated to 50° C. and stirred to dissolve completely. A solution of methanesulfonic acid (156 mg, 1.62 mmol) in ethyl acetate (1 mL) was added to the flask. After the addition, the mixture was stirred for 30 min while keeping the temperature. Then the heating was stopped, the solution was cooled to room temperature, and continuously stirred for 24 h. The resulting solution was filtered, the filter cake was washed with ethyl acetate (3 mL) and then dried at 60° C. for 12 h under vacuum to obtain a yellow solid (0.39 g, 68%).

Results Identification:

¹H NMR (400 MHz, CH₃OH-d₄) δ 8.02 (d, J=3.1 Hz, 1H), 7.93 (d, J=3.1 Hz, 1H), 7.57 (dd, J=8.7, 6.0 Hz, 1H), 7.41 (t, J=8.3 Hz, 1H), 7.30 (dd, J=8.6, 2.5 Hz, 1H), 7.18-7.11 (m, 3H), 6.22 (s, 1H), 4.81 (d, J=16.1 Hz, 1H), 4.62 (d, J=16.0 Hz, 1H), 4.43-4.34 (m, 1H), 4.18 (dd, J=14.7, 3.2 Hz, 1H), 4.05 (t, J=9.0 Hz, 1H), 3.90-3.80 (m, 2H), 3.67 (s, 3H), 3.65-3.54 (m, 2H), 3.42-3.29 (m, 2H), 2.94 (t, J=7.4 Hz, 2H), 2.69 (s, 3H), 2.64 (t, J=7.4 Hz, 2H).

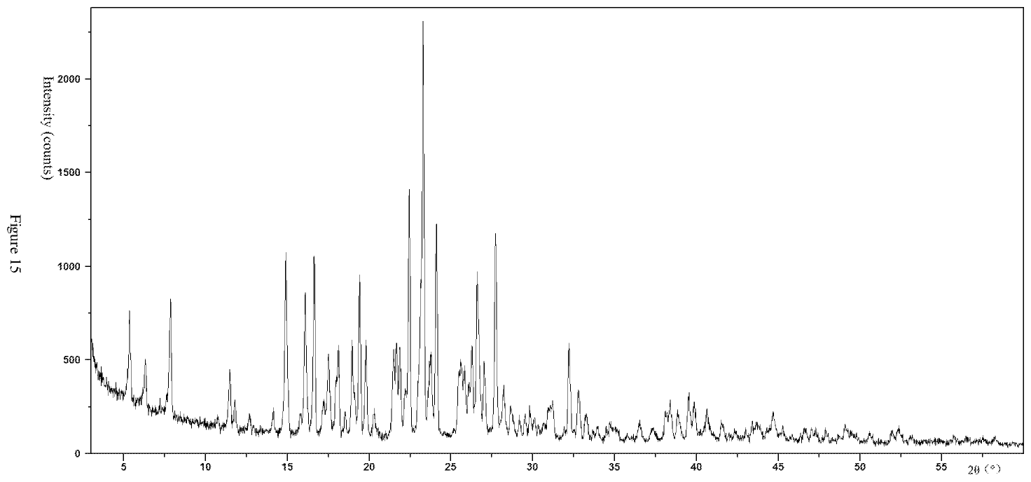

(2) Analysis and identification through Empyrean X-ray powder diffraction (XRPD): The obtained XRPD pattern is shown in FIG. 15. The X-ray powder diffraction pattern of the methanesulfonate crystal form A contains the diffraction peaks with 2θ angles of 5.34°, 6.29°, 7.82°, 10.73°, 11.46°, 11.78°, 12.67°, 14.12°, 14.89°, 15.77°, 16.08°, 16.62°, 17.19°, 17.49°, 18.04°, 18.51°, 18.96°, 19.39°, 19.78°, 20.28°, 21.46°, 21.64°, 21.85°, 22.41°, 23.25°, 23.72°, 24.08°, 25.53°, 25.80°, 26.23°, 26.60°, 27.01°, 27.68°, 27.69°, 28.18°, 28.66°, 29.51°, 29.80°, 30.07°, 31.04°, 32.19°, 32.77°, 33.23°, 33.91°, 34.87°, 36.49°, 37.30°, 38.09°, 38.36°, 38.85°, 39.50° and 39.83°, and the diffraction peaks have an error tolerance of ±0.2°.

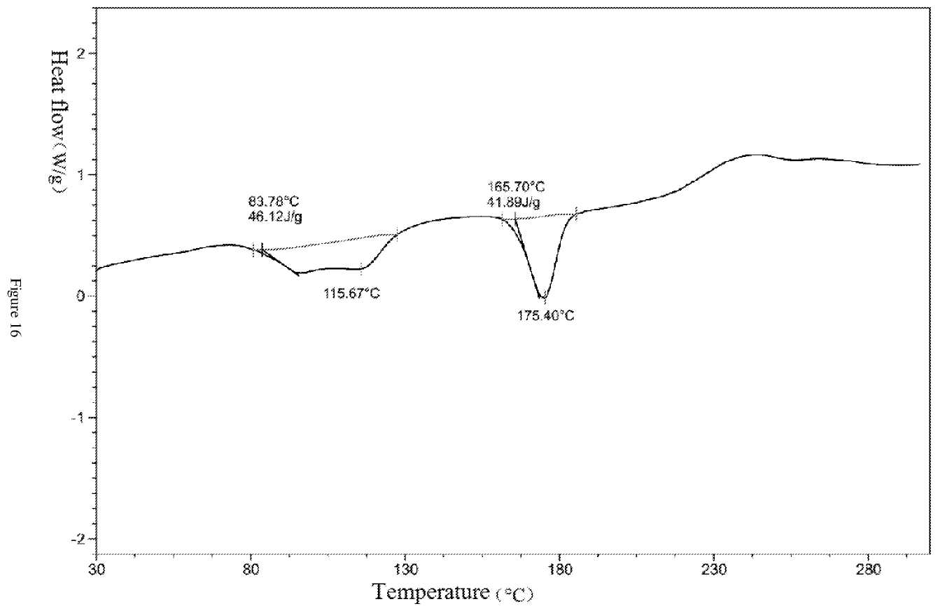

(3) Analysis and identification through TA Q2000 Differential Scanning Calorimetry (DSC): the scanning speed is 10° C./min, and the obtained DSC pattern is shown in FIG. 16, which contains endothermic peaks of 115.67° C. and 175.40° C. There is an error tolerance of ±3° C.

### Example 9

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)prop ionic acid p-toluenesulfonate crystal form A

Preparation:

The compound having Formula (I) (0.5 g, 0.75 mmol), ethyl acetate (5 mL) and water (0.25 mL) were added in sequence into a dry reaction flask, the mixture was stirred and dissolved completely at room temperature and then heated to 50° C. A solution of p-toluenesulfonic acid monohydrate (155 mg, 0.81 mmol) in ethyl acetate (1 mL) was added to the flask. After the addition, the mixture was stirred for 30 min while keeping the temperature. Then the heating was stopped, the solution was cooled to room temperature, and continuously stirred for 15 h. The resulting solution was filtered, the filter cake was washed with ethyl acetate (2 mL) and then dried at 60° C. for 12 h under vacuum to obtain a yellow solid (0.48 g, 76.1%).

Results Identification:

(1) NMR: ¹H NMR (400 MHz, CH₃OH-d₄) δ 8.00 (d, J=3.1 Hz, 1H), 7.89 (d, J=3.1 Hz, 1H), 7.68 (d, J=8.1 Hz, 2H), 7.55 (dd, J=8.7, 6.0 Hz, 1H), 7.43-7.39 (m, 1H), 7.28 (dd, J=8.6, 2.5 Hz, 1H), 7.17 (d, J=8.0 Hz, 2H), 7.15-7.04 (m, 3H), 6.18 (s, 1H), 4.77 (d, J=16.1 Hz, 1H), 4.61 (d, J=16.1 Hz, 1H), 4.44-4.36 (m, 1H), 4.16 (dd, J=14.7, 3.1 Hz, 1H), 4.01 (t, J=9.0 Hz, 1H), 3.90-3.80 (m, 2H), 3.65 (s, 3H), 3.63-3.56 (m, 2H), 3.42-3.25 (m, 2H), 2.94 (t, J=7.4 Hz, 2H), 2.63 (t, J=7.4 Hz, 2H), 2.34 (s, 3H).

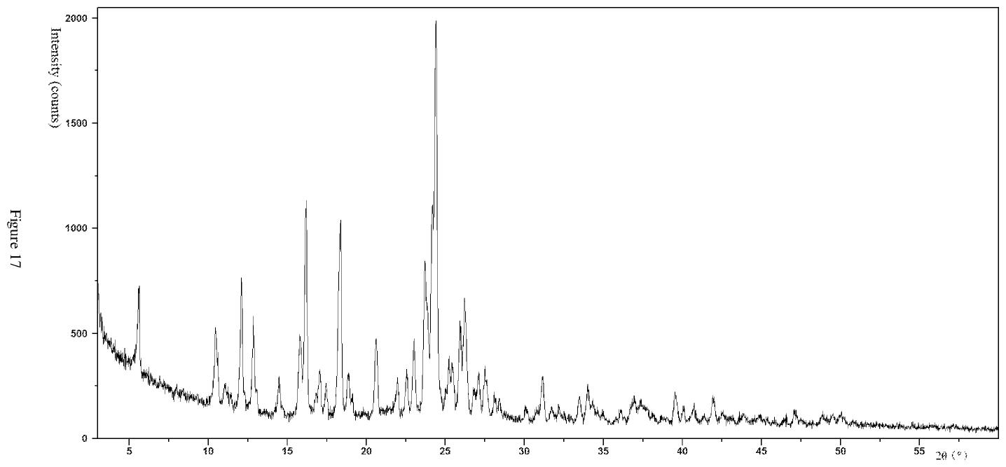

(2) Analysis and identification through Empyrean X-ray powder diffraction (XRPD): The obtained XRPD pattern is shown in FIG. 17. The X-ray powder diffraction pattern of the p-toluenesulfonate crystal form A contains the diffraction peaks with 2θ angles of 5.57°, 10.46°, 11.10°, 12.08°, 12.84°, 14.46°, 15.79°, 16.15°, 17.01°, 17.44°, 18.30°, 18.85°, 20.59°, 21.92°, 22.53°, 22.98°, 23.70°, 24.15°, 24.37°, 25.20°, 25.43°, 25.91°, 26.20°, 26.79°, 27.06°, 27.51°, 28.12°, 30.07°, 31.10°, 31.75°, 33.44°, 34.04°, 36.05°, 37.21° and 39.52°, and the diffraction peaks have an error tolerance of ±0.2°.

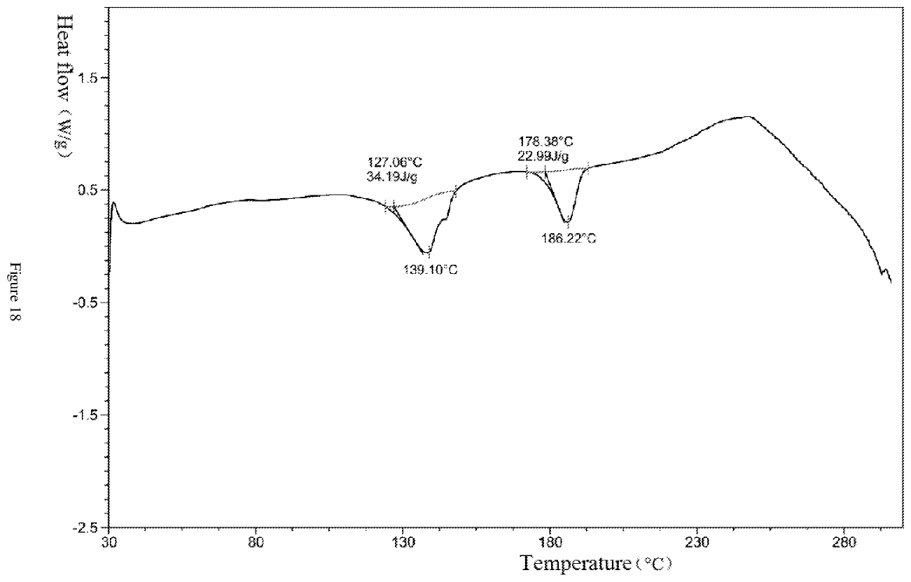

(3) Analysis and identification through TA Q2000 Differential Scanning Calorimetry (DSC): the scanning speed was 10° C./min, and the obtained DSC pattern was shown in FIG. 18, which contains endothermic peaks of 139.10° C. and 186.22° C. There is an error tolerance of ±3° C.

### Example 10

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluoro phenyl)propionic acid benzenesulfonate crystal form A

Preparation:

The compound having Formula (I) (0.5 g, 0.75 mmol), ethyl acetate (5 mL) and water (0.25 mL) were added in sequence into a dry reaction flask, the mixture was stirred and dissolved completely at room temperature and then heated to 50° C. A solution of benzenesulfonic acid (130 mg, 0.82 mmol) in ethyl acetate (1 mL) was added to the flask. After the addition, the mixture was stirred for 30 min while keeping the temperature. Then the heating was stopped, the solution was cooled to room temperature, and continuously stirred for 24 h. The resulting solution was filtered, the filter cake was washed with ethyl acetate (2 mL) and then dried at 60° C. for 12 h under vacuum to obtain a yellow solid (0.47 g, 75.4%).

Results Identification:

(1) NMR: ¹H NMR (400 MHz, CH₃OH-d₄) δ 7.99 (d, J=3.1 Hz, 1H), 7.89 (d, J=3.1 Hz, 1H), 7.84-7.77 (m, 2H), 7.55 (dd, J=8.7, 6.0 Hz, 1H), 7.44-7.32 (m, 4H), 7.28 (dd, J=8.6, 2.5 Hz, 1H), 7.15-7.06 (m, 3H), 6.18 (s, 1H), 4.78 (d, J=16.1 Hz, 1H), 4.61 (d, J=16.1 Hz, 1H), 4.45-4.36 (m, 1H), 4.16 (dd, J=14.7, 3.1 Hz, 1H), 4.01 (t, J=9.0 Hz, 1H), 3.90-3.80 (m, 2H), 3.65 (s, 3H), 3.64-3.54 (m, 2H), 3.40-3.28 (m, 2H), 2.94 (t, J=7.4 Hz, 2H), 2.63 (t, J=7.4 Hz, 2H).

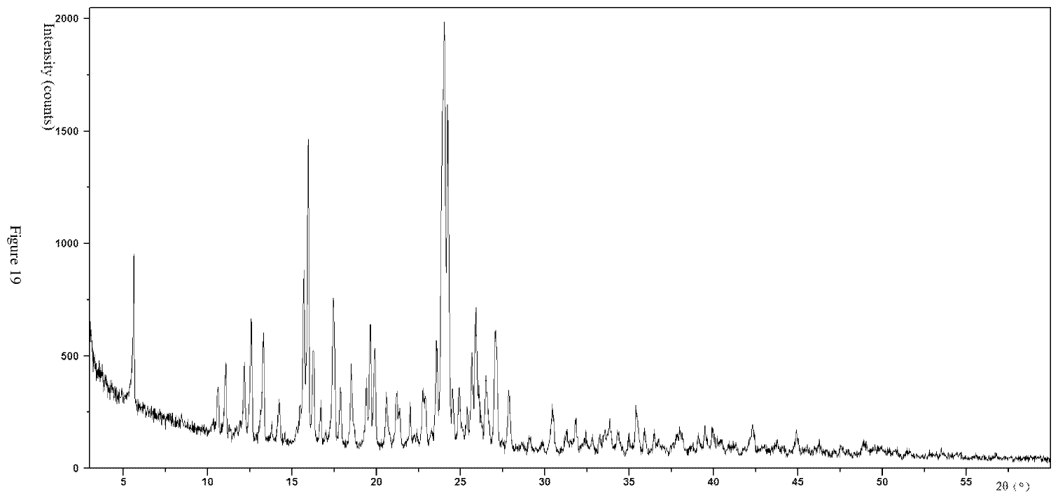

(2) Analysis and identification through Empyrean X-ray powder diffraction (XRPD): The obtained XRPD pattern is shown in FIG. 19. The X-ray powder diffraction pattern of the benzenesulfonate crystal form A contains the diffraction peaks with 2θ angles of 5.59°, 10.58°, 11.04°, 12.15°, 12.55°, 13.27°, 13.78°, 14.21°, 15.68°, 15.93°, 16.24°, 16.68°, 17.44°, 17.84°, 18.50°, 19.39°, 19.61°, 19.88°, 20.59°, 21.22°, 21.98°, 22.75°, 22.89°, 23.55°, 23.88°, 24.02°, 24.22°, 24.51°, 24.89°, 25.36°, 25.63°, 25.88°, 26.50°, 27.05°, 27.84°, 29.07°, 29.79°, 30.40°, 31.24°, 31.79°, 32.36°, 32.77°, 33.22°, 33.75°, 34.31°, 34.95°, 35.40°, 35.88°, 36.46°, 37.93°, 39.08°, 39.47° and 39.91°, and the diffraction peaks have an error tolerance of ±0.2°.

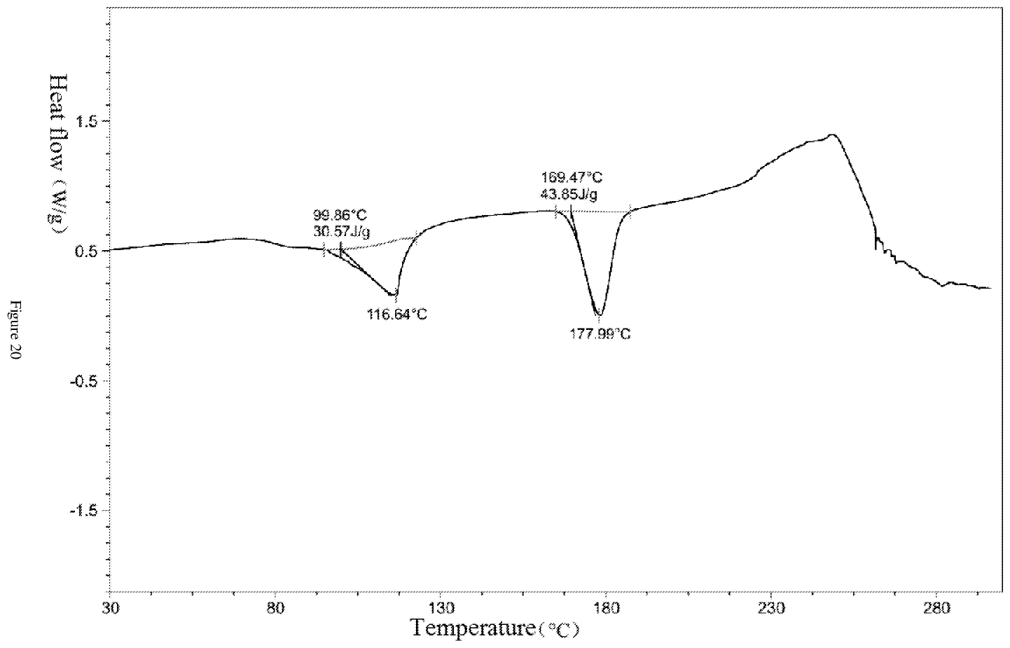

(3) Analysis and identification through TA Q2000 Differential Scanning Calorimetry (DSC): the scanning speed is 10° C./min, and the obtained DSC pattern is shown in FIG. 20, which contains endothermic peaks of 116.64° C. and 177.99° C. There is an error tolerance of ±3° C.

### Example 11

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluoro phenyl)propionic acid hydrobromide crystal form A

Preparation:

The compound having Formula (I) (0.5 g, 0.75 mmol), acetone (5 mL), and water (0.2 mL) were added in sequence into a dry reaction flask, the mixture was stirred and dissolved completely at room temperature and then heated to 50° C. A solution of hydrobromic acid (0.17 g, 0.84 mmol, 40%) in acetone (0.5 mL) was added to the flask. After the addition, the mixture was stirred for 30 min while keeping the temperature. Then the heating was stopped, the solution was cooled to room temperature, and continuously stirred for 12 h. The resulting solution was filtered, the filter cake was washed with acetone (5 mL) and then dried at 60° C. for 12 h under vacuum to obtain a yellow solid (0.41 g, 73%).

Results Identification:

(1) Ion Chromatography

The salt formation ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophen yl)propionic acid hydrobromide crystal form A with hydrobromic acid was determined by ion chromatography (TI-00375). The method parameters were shown in the table below.

| Instrument model | | TI-00375 | | |
|---|---|---|---|---|
| Method | Eluent | 4.5 mM Na₂CO₃ + 0.8 mM NaHCO₃ | | |
| parameters | Flow rate | 1.0 mL/min | Column | 30° C. |
| | | | temperature | |
| | Column | IonPac AS23, | Specification | 4 × 250 mm, Serial |
| | | DIONEX | | No. 180522257 |
| | Quantitative | 100 μL | Detector | Suppressive conductivity |
| | loop | | | detector |

| | | |
|---|---|---|
| Sample | Control | 5 mL of Br⁻ stock solution (10 μg/mL) was transferred to a 50 mL |
| test | solution | volumetric flask, ultrapure water was added to complete the volume, |
| | preparation | then shaken well. |
| | Sample | The sample was precisely weighed about 25 mg and added into a 25 mL |
| | solution | volumetric flask, then 4 mL of methanol was added to the flask to |
| | preparation | dissolve the sample. 200 μL of 450 mM Na₂CO₃ solution was added |
| | | into the mixture, ultrapure water was added to complete the volume, |
| | | then shaken well. The mixture was stood to clear and then made |
| | | constant volume. 1 mL of the above solution was transferred into a 100 |
| | | mL volumetric flask. Ultrapure water was added to complete the |
| | | volume, then shaken well. |

The test results show that the salt formation molar ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophen yl)propionic acid hydrobromide crystal form A with hydrobromic acid is 1:1.

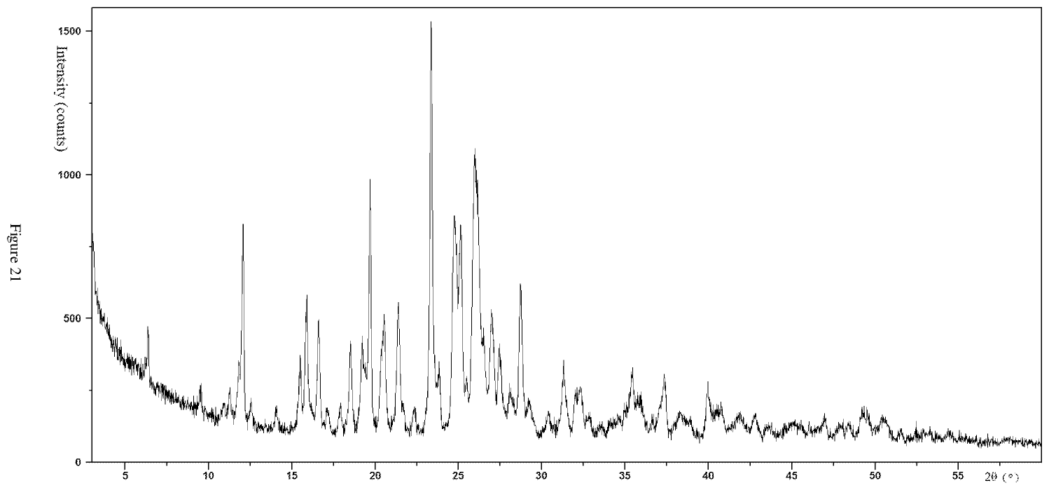

(2) Analysis and identification through Empyrean X-ray powder diffraction (XRPD): The obtained XRPD pattern is shown in FIG. 21. The X-ray powder diffraction pattern of the Hhydrobromide crystal form A contains the diffraction peaks with 2θ angles of 6.34°, 9.50°, 11.25°, 12.03°, 12.54°, 14.05°, 15.46°, 15.85°, 16.58°, 17.13°, 17.87°, 18.50°, 19.28°, 19.67°, 20.45°, 21.37°, 22.31°, 23.33°, 23.75°, 24.74°, 25.09°, 25.92°, 26.15°, 26.48°, 26.98°, 27.44°, 28.09°, 28.70°, 29.24°, 30.35°, 31.29°, 31.98°, 32.27°, 32.77°, 35.37°, 35.88°, 37.25°, 38.44° and 39.93°, and the diffraction peaks have an error tolerance of ±0.2°.

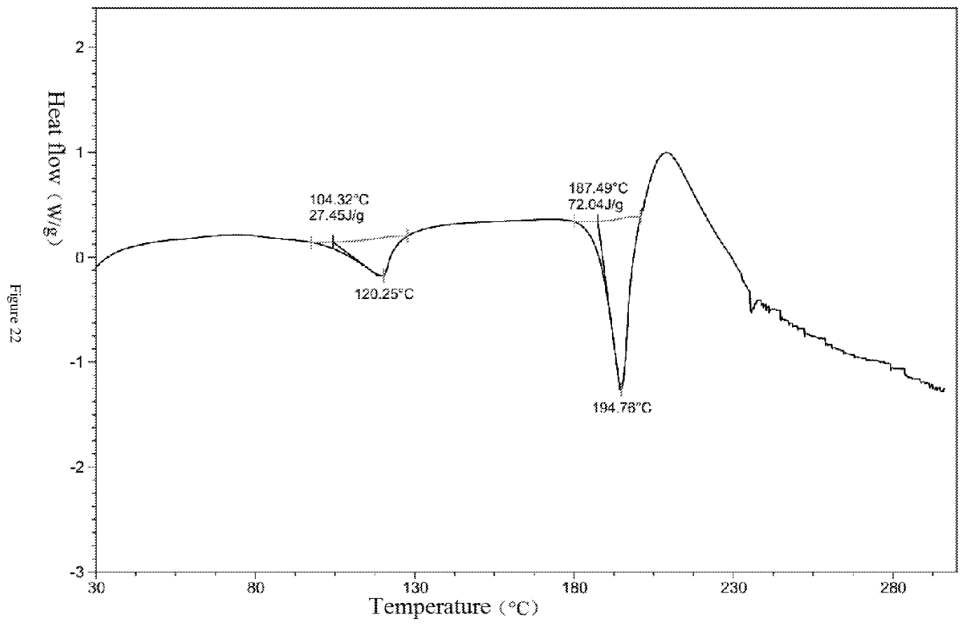

(3) Analysis and identification through TA Q2000 Differential Scanning Calorimetry (DSC): the scanning speed was 10° C./min, and the obtained DSC pattern was shown in FIG. 22, which contains endothermic peaks of 120.25° C. and 194.76° C. There is an error tolerance of ±3° C.

### Example 12

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluoro phenyl)propionic acid hydrochloride crystal form B

Preparation:

The compound having Formula (I) (1.00 g, 1.49 mmol), acetone (9 mL) and water (0.5 mL) were added in sequence into a reaction flask, then the mixture was heated to 50° C. Concentrated hydrochloric acid (441 mg, 4.48 mmol, 37%) was diluted with acetone (1 mL), and the mixture was added to the flask. After the addition, the mixture was stirred for 20 min while keeping the temperature. Then the heating was stopped, the solution was cooled to room temperature. The mixture was continuously stirred for 20 h. The resulting solution was filtered, the filter cake was washed with acetone (10 mL) and then dried at 60° C. for 12 h under vacuum to obtain a yellow solid (976 mg, 88%).

Results Identification:

(1) Ion Chromatography

The salt formation ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophen yl)propionic acid hydrochloride crystal form B with hydrochloric acid was determined by ion chromatography (TI-00375). The method parameters were shown in the table below.

| Instrument model | | TI-00375 | | |
|---|---|---|---|---|
| Method | Eluent | 4.5 mM Na₂CO₃ + 0.8 mM NaHCO₃ | | |
| parameters | Flow rate | 1.0 mL/min | Column | 30° C. |
| | | | temperature | |
| | Column | IonPac AS23, DIONEX | Specification | 4 × 250 mm, Serial |
| | | | | No. 180522257 |
| | Quantitative | 100 μL | Detector | Suppressive conductivity |
| | loop | | | detector |

| | | |
|---|---|---|
| Sample | Control | 5 mL of Cl⁻ stock solution (10 μg/mL) was transferred to a 50 mL |
| test | solution | volumetric flask. Ultrapure water was added to complete the volume, |
| | preparation | then shaken well. |
| | | 1. The sample was precisely weighed about 25 mg and added into a 25 |
| | Sample | mL volumetric flask, then 4 mL of methanol was added to the flask to |
| | solution | dissolve the sample. 200 μL of 450 mM Na₂CO₃ solution was added |
| | preparation | into the mixture, ultrapure water was added to complete the volume, |
| | | then shaken well. The mixture was stood to clear and then made |
| | | constant volume. 2. 1 mL of the above solution was transferred into a |
| | | 50 mL volumetric flask. Ultrapure water was added to complete the |
| | | volume, then shaken well. |

The test results show that the salt formation molar ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophen yl)propionic acid hydrochloride crystal form B with hydrochloric acid is 1:2.

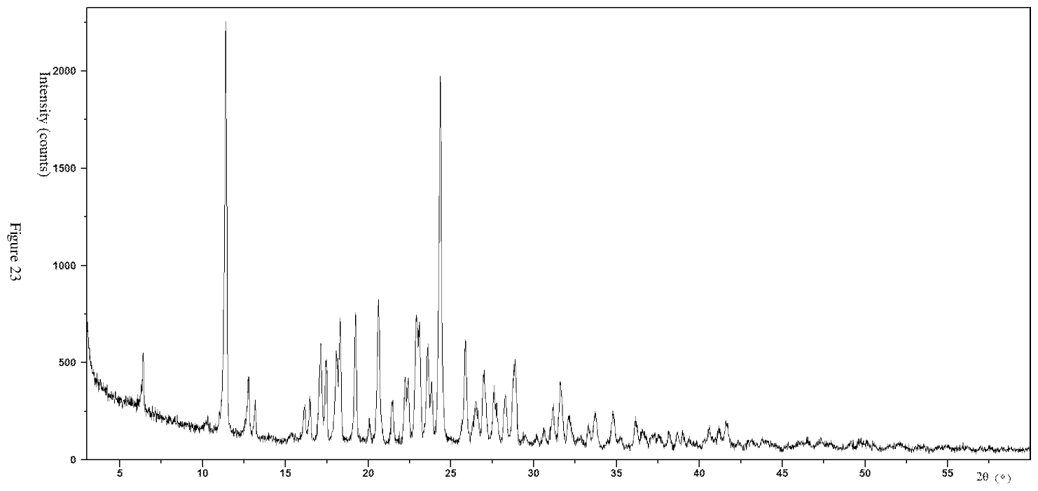

(2) Analysis and identification through Empyrean X-ray powder diffraction (XRPD): The obtained XRPD pattern is shown in FIG. 23. The X-ray powder diffraction pattern of the hydrochloride crystal form B contains the diffraction peaks with 2θ angles of 6.38°, 10.23°, 11.37°, 12.73°, 13.14°, 16.13°, 16.45°, 17.10°, 17.43°, 18.06°, 18.28°, 19.20°, 20.04°, 20.59°, 21.43°, 22.21°, 22.39°, 22.88°, 23.07°, 23.56°, 23.80°, 24.32°, 25.84°, 26.47°, 26.97°, 27.61°, 28.25°, 28.80°, 29.41°, 30.58°, 31.11°, 31.59°, 32.10°, 32.77°, 33.28°, 33.67°, 34.75°, 35.21°, 36.12°, 36.55°, 37.28°, 38.13°, 38.640 and 38.97°, and the diffraction peaks have an error tolerance of ±0.2°.

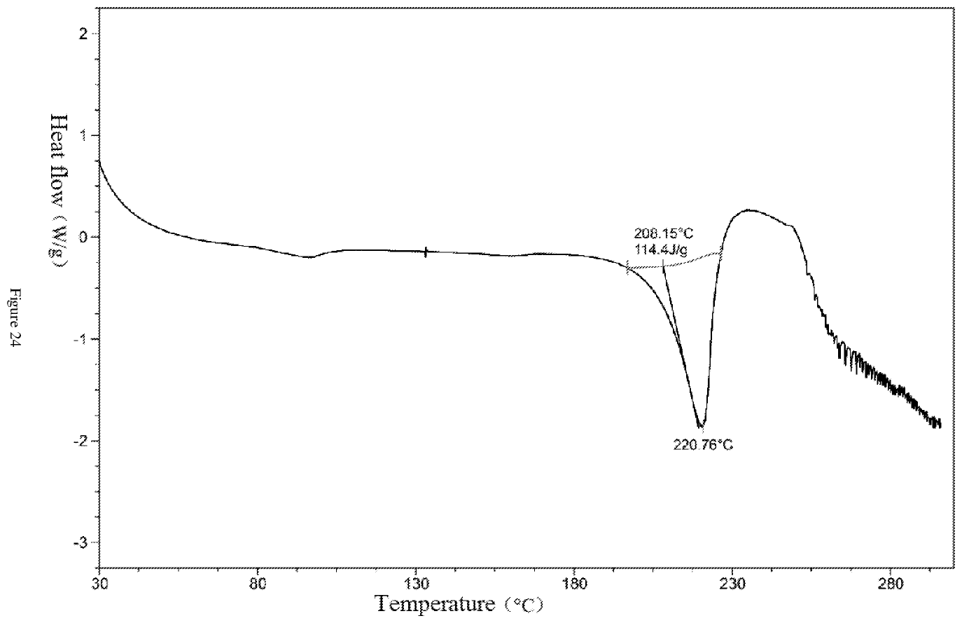

(3) Analysis and identification through TA Q2000 Differential Scanning Calorimetry (DSC): the scanning speed is 10° C./min, and the obtained DSC pattern is shown in FIG. 24, which contains an endothermic peak of 220.76° C. There is an error tolerance of ±3° C.

### Example 13

### 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)prop ionic acid phosphate crystal form C

Preparation:

The compound having Formula (I) (5 g, 7.45 mmol) and acetone (75 mL) were added in sequence into a reaction flask, the mixture was stirred to completely dissolved at room temperature, and then heat to 50° C. To the resulting mixture was added phosphoric acid aqueous solution (2.6 g, 23 mmol, 85%) diluted with water (0.5 mL). After the addition, the mixture was stirred for 1 h while keeping the temperature. Then the heating was stopped, the solution was cooled to room temperature. The mixture was continuously stirred for 24 h. The resulting solution was filtered, the filter cake was washed with acetone (20 mL) and then dried at 60° C. for 12 h under vacuum to obtain a yellow solid (4.9 g, 76%).

Results Identification:

(1) Ion Chromatography

The salt formation ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid phosphate crystal form C with phosphoric acid was determined by ion chromatography (TI-00375). The method parameters were shown in the table below.

| Instrument model | | TI-00375 | | |
|---|---|---|---|---|
| Method | Eluent | 4.5 mM Na₂CO₃ + 0.8 mM NaHCO₃ | | |
| parametesr | Flow rate | 1.0 mL/min | Column | 30° C. |
| | | | temperature | |
| | Column | IonPac AS23, DIONEX | Specification | 4 × 250 mm, Serial |
| | | | | No. 180522257 |
| | Quantitative | 100 μL | Detector | Suppressive conductivity |
| | loop | | | detector |

| | | |
|---|---|---|
| Sample | Control | 5 mL of PO₄³⁻ stock solution (10 μg/mL) was transferred to a 50 mL |
| test | solution | volumetric flask. Ultrapure water was added to complete the volume, |
| | preparation | then shaken well. |
| | Sample | The sample was precisely weighed about 22 mg and added into a 50 mL |
| | solution | volumetric flask, then 4 mL of methanol was added to the flask to |
| | preparation | dissolve the sample. 200 μL of 450 mM Na₂CO₃ solution was added |
| | | into the mixture, ultrapure water was added to complete the volume, |
| | | then shaken well. The mixture was stood to clear and then made |
| | | constant volume. 1 mL of the above solution was transferred into a 100 |
| | | mL volumetric flask. Ultrapure water was added to complete the |
| | | volume, then shaken well. |

The test results show that the salt formation molar ratio of the compound having Formula (Ia) in 3-(4-((S)-7-(((R)-6-(2-chloro-4-fluorophenyl)-5-(methoxycarbonyl)-2-(thiazol-2-yl)-1,6-dihydropyrimidin-4-yl)methyl)-3-oxohexahydroimidazo[1,5-a]pyrazine-2(3H)-yl)-3-fluorophenyl)propionic acid phosphate crystal form C with phosphoric acid is 1:2.

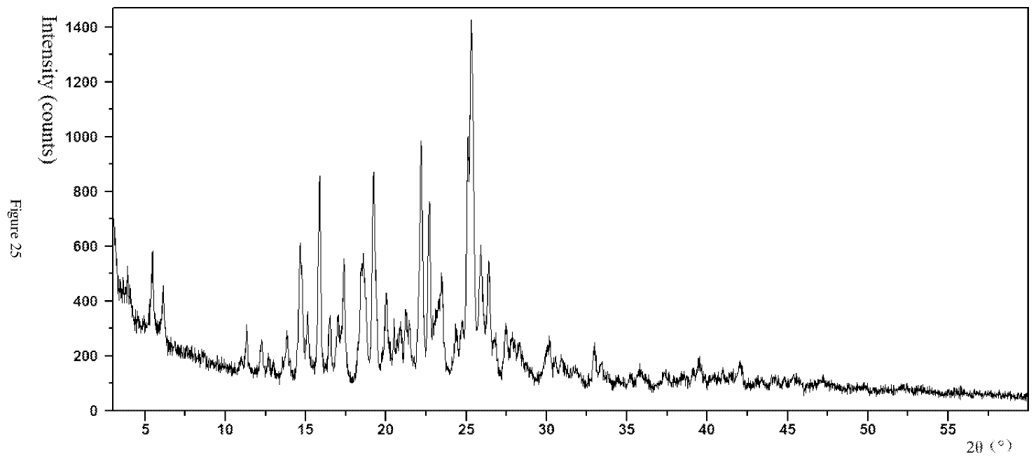

(2) Analysis and identification through Empyrean X-ray powder diffraction (XRPD): The obtained XRPD pattern is shown in FIG. 25. The X-ray powder diffraction pattern of the phosphate crystal form C contains the diffraction peaks with 2θ angles of 3.91°, 5.44°, 6.11°, 11.30°, 12.23°, 13.82°, 14.67°, 15.10°, 15.83°, 16.49°, 17.00°, 17.35°, 18.47°, 18.68°, 19.22°, 20.00°, 20.49°, 20.87°, 21.21°, 21.43°, 22.15°, 22.67°, 23.29°, 24.34°, 24.70°, 25.05°, 25.30°, 25.88°, 26.37°, 26.76°, 27.44°, 28.02°, 30.06°, 30.86°, 32.97°, 35.19°, 35.82°, 37.31°, 39.38°, 41.99°, 45.36° and 47.13°, and the diffraction peaks have an error tolerance of ±0.2°.

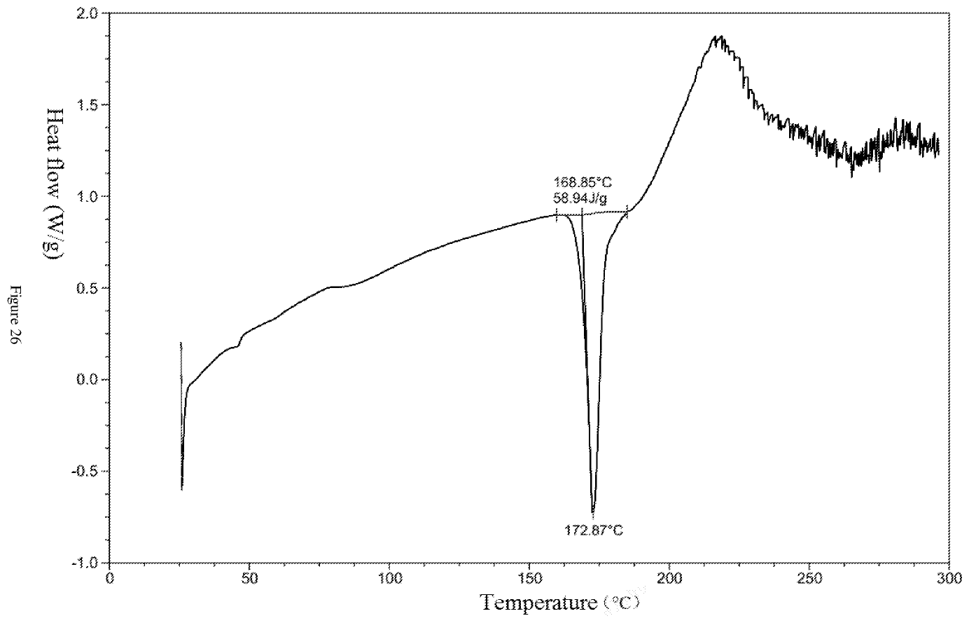

(3) Analysis and identification through TA Q2000 Differential Scanning Calorimetry (DSC): the scanning speed is 10° C./min, and the obtained DSC pattern is shown in FIG. 26, which contains an endothermic peak of 172.9° C. There is an error tolerance of ±3° C.

2. Examples of Property Testing

1. Stability Test

High temperature test: An appropriate amount of the test product was added into a flat weighing bottle and spread into a thin layer with thickness ≤5 mm. The bottle was placed at 60° C. or 40° C. for 10 days. Samples were taken at the 5th and 10th days to check the appearance, related substances and purity. If the test product changes significantly at 60° C., the test is performed in the same way at 40° C. If the test product does not change significantly at 60° C., the test at 40° C. is not necessary.

High humidity test: An appropriate amount of the test product was added into a flat weighing bottle and spread into a thin layer with thickness 5 mm. The bottle was placed at 25° C. and a relative humidity of 90%±5% for 10 days. Samples were taken at the 5th and 10th days to check the appearance, related substances and purity. At the same time, the weight of the test product before and after the test were accurately weighed to investigate the moisture absorption and deliquescence performance of the test product. Light test: An appropriate amount of the test product was added into a flat weighing bottle and spread into a thin layer with thickness ≤5 mm. The bottle was opened and placed in a light box (with UV) at the illuminance 4500±500 lx, UV light≥0.7 w/m² for 10 days. Samples were taken at the 5th and 10th days to check the appearance, related substances and purity. Results are shown in table 2:

TABLE 2
Stability experimental results of the test products

| | | | High temperature | | High humidity | | | |
|---|---|---|---|---|---|---|---|---|
| Test | Condition | | (60° C.) | | (90% RH) | | Light | |
| product | Project | 0 days | 5 days | 10 days | 5 days | 10 days | 5 days | 10 days |
| Compound | Appearance | Yellow | Yellow | Yellow | Yellow | Yellow | Yellow | Yellow |
| having | | powder | powder | powder | powder | powder | powder | powder |
| Formula | | | partially | partially | | | | |
| (I) | | | melted | melted | | | | |
| | Purity (%) | 98.61 | 95.40 | 94.07 | 98.66 | 98.75 | 98.66 | 98.20 |
| Phosphate | Appearance | Yellow | Yellow | Yellow | Yellow | Yellow | Yellow | Yellow |
| crystal form | | powder | powder | powder | powder | powder | powder | powder |
| A of the | Purity (%) | 99.13 | 98.86 | 98.39 | 99.04 | 99.07 | 99.03 | 99.06 |
| compound having | | | | | | | | |
| Formula | | | | | | | | |
| (Ia) | | | | | | | | |
| Phosphate crystal | Appearance | Yellow | Yellowish | Brown | Yellow | Yellow | Yellow | Yellow |
| form C of the | | powder | brown solid | solid | powder | powder | powder | powder |
| compound having | Purity (%) | 99.34 | 87.42 | 60.31 | 99.29 | 99.36 | 99.30 | 99.30 |
| Formula | | | | | | | | |
| (Ia) | | | | | | | | |
| Hydrobromide | Appearance | Yellow | Yellow | Yellow | Yellow | Yellow | Yellow | Yellow |
| crystal | | powder | powder | powder | powder | powder | powder | powder |
| form A of the | Purity (%) | 98.41 | 98.43 | 98.93 | 98.82 | 98.69 | 98.80 | 98.88 |
| compound having | | | | | | | | |
| Formula | | | | | | | | |
| (Ia) | | | | | | | | |
| Methanesulfonate | Appearance | Yellow | Yellow | Yellow | Yellow | Yellow | Yellow | Yellow |
| crystal | | powder | powder | powder | powder | powder | powder | powder |
| form A of the | Purity (%) | 99.40 | 99.15 | 98.68 | 99.37 | 99.36 | 99.37 | 99.40 |
| compound having | | | | | | | | |
| Formula | | | | | | | | |
| (Ia) | | | | | | | | |
| Sulfate crystal | Appearance | yellow | yellow | yellow | yellow | yellow | yellow | yellow |
| form B of the | | solid | solid | solid | solid | solid | solid | solid |
| compound having | Purity (%) | 98.98 | 98.94 | 98.68 | 98.90 | 98.94 | 99.01 | 98.91 |
| Formula | | | | | | | | |
| (Ia) | | | | | | | | |
| Sulfate crystal | Appearance | yellow | yellow | yellow | yellow | yellow | yellow | yellow |
| form A of the | | solid | solid | solid | solid | solid | solid | solid |
| compound having | Purity (%) | 99.14 | 99.16 | 99.12 | 99.17 | 99.22 | 99.17 | 99.15 |
| Formula | | | | | | | | |
| (Ia) | | | | | | | | |
| Hydrochloride | Appearance | Yellow | Yellow | Yellow | Yellow | Yellow | Yellow | Yellow |
| crystal | | powder | powder | powder | powder | powder | powder | powder |
| form A of the | Purity (%) | 99.39 | 99.36 | 99.22 | 99.37 | 99.30 | 99.34 | 99.26 |
| compound having | | | | | | | | |
| Formula | | | | | | | | |
| (Ia) | | | | | | | | |
| L-arginine | Appearance | Yellow | Yellow | Yellow | Yellow | Yellow | Yellow | Yellow |
| salt crystal | | powder | powder | powder | powder | powder | powder | powder |
| form A of the | | | | | | | | |
| compound having | Purity (%) | 99.52 | 99.46 | 99.42 | 99.38 | 99.39 | 99.10 | 98.72 |
| Formula | | | | | | | | |
| (I) | | | | | | | | |
| N,N-dimethyl- | Appearance | Light | Light | Light | Light | Light | Light | Light |
| formamide | | yellow | yellow | yellow | yellow | yellow | yellow | yellow |
| complex of the | | powder | powder | powder | powder | powder | powder | powder |
| compound having | Purity (%) | 98.63 | 98.48 | 97.76 | 98.87 | 98.71 | 98.82 | 98.48 |
| Formula | | | | | | | | |
| (I) | | | | | | | | |

It can be found from the data in the above table that after the Phosphate crystal form A of the compound having Formula (Ia), the Hydrobromide crystal form A of the compound having Formula (Ia), the Methanesulfonate crystal form A of the compound having Formula (Ia), the sulfate crystal form B of the compound having Formula (Ia), the sulfate crystal form A of the compound having Formula (Ia), the hydrochloride crystal form A of the compound having Formula (Ia), the arginine salt crystal form A of the compound having Formula (I), and N,N-dimethylformamide complex of the compound having Formula (I) are placed under high temperature, high humidity or light conditions for 10 days, the appearances have no change and the impurity contents are almost no increase, and the stability are very good. The Phosphate crystal form C of the compound having Formula (Ia) and Compound having Formula (I) are unstable under high temperature.

2. Pharmacokinetic Evaluation of Experimental Animals after Oral Administration of Quantitative Test Samples

1. Test Method:

Beagle dogs were administered orally via capsules with the test samples at doses of 2.5 mg/kg, 5 mg/kg or 10 mg/kg. Blood samples were taken at 0.25, 0.5, 1, 2, 4, 6, 8 and 24 hours from forelimb vein after the administration, and collected in anticoagulation tube with EDTA-K2. After liquid-liquid extraction, the blood samples were quantitatively analyzed on a triple quadrupole tandem mass spectrometer using multiple reactive ion monitoring (MRM). Pharmacokinetic parameters (AUC₀₋ₜ and Cₘₐₓ) were calculated using a noncompartmental method by WinNonLin 6.3 software.

Results are shown in table 3:

TABLE 3
Pharmacokinetic parameters of the compound having
Formula (I) and the salt of the compound having
Formula (I) or Formula (Ia) in beagle dogs

| | Administration | AUC₀₋ₜ | Cmax |
|---|---|---|---|
| Salt type | dosage (mg/kg) | (h*ng/ml) | (ng/ml) |
| Compound having | 10 | 6340 | 3720 |
| Formula (I) | | | |
| Sulfate crystal form B | 10 | 10840 | 6620 |
| of the compound | | | |
| having Formula (Ia) | | | |
| L-arginine salt crystal | 10 | 13100 | 6810 |
| form A of the | | | |
| compound having | | | |
| Formula (I) | | | |
| Hydrochloride crystal | 10 | 8630 | 4980 |
| form A of the | | | |
| compound having | | | |
| Formula (Ia) | | | |
| Phosphate crystal | 10 | 1900 | 907 |
| form C of the | | | |
| compound having | | | |
| Formula (Ia) | | | |
| Phosphate crystal | 10 | 4350 | 2690 |
| form A of the | | | |
| compound having | | | |
| Formula (Ia) | | | |
| Hydrobromide crystal | 10 | 5690 | 3340 |
| form A of the | | | |
| compound having | | | |
| Formula (Ia) | | | |

The experimental results show that the sulfate crystal form B of the compound having Formula (Ia), the L-arginine salt crystal form A of the compound having Formula (I) and the hydrochloride crystal form A of the compound having Formula (Ia) of the present invention have better pharmacokinetic properties in the experimental animals, specifically higher exposure, which indicates that the sulfate crystal form B of the compound having Formula (Ia), the L-arginine salt crystal form A of the compound having Formula (I) and the hydrochloride crystal form A of the compound having Formula (Ia) of the present invention are better absorbed in animals.

3. Experimental Research on Hygroscopicity

A dry stoppered glass weighing bottle (outer diameter was 50 mm, height was 15 mm) was placed in a suitable constant temperature dryer (a saturated solution of ammonium chloride or ammonium sulfate was placed at the bottom, the relative humidity was within 90%±2%) at 25° C.±1° C. the day before, and the weighing bottle was precisely weighed (m₁). An appropriate amount of the test product was spread flat in the above weighing bottle. The thickness of the test product was generally about 1 mm, and the weighing bottle was precisely weighed (m₂). The weighing bottle was opened and placed for 24 hours under the above constant temperature and humidity conditions with the bottle cap. The weighing bottle was closed and precisely weighed (m₃), and the weight gain percentage (%) was calculated.

Testing method: According to Ph. Eur. <5.11>; Ch.P. 2015IV General Rule 9103;

$$Weight gain rate of moisture absorption ( \% ) = \frac{m 3 - m 2}{m 2 - m 1} \times 100 \%$$

The hygroscopic feature: weight gain rate of moisture absorption

Judgment of hygroscopicity results:
(1) Deliquescence: absorb enough water to form a liquid;
(2) Highly hygroscopicity: not less than 15%;
(3) Hygroscopicity: less than 15% but not less than 2%;
(4) Slightly hygroscopicity: less than 2% but not less than 0.2%;
(5) No or almost no hygroscopicity: less than 0.2%.

TABLE 4
Experimental results of hygroscopicity of the salt of
the compound having Formula (I) or (Ia)

| | Weight gain of |
|---|---|
| | moisture |
| Test sample | absorption (%) |
| Hydrochloride crystal form A of the | 1.32 |
| compound having Formula (Ia) | |
| Sulfate crystal form B of the compound | 0.97 |
| having Formula (Ia) | |
| L-arginine salt form A of the compound | 1.34 |
| having Formula (I) | |

The experimental results show that the hydrochloride crystal form A of the compound having Formula (Ia), the sulfate crystal form B of the compound having Formula (Ia) and the L-arginine salt crystal form A of the compound having Formula (I) are slightly hygroscopicity.

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the above terms throughout this specification are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1.-28. (canceled)

29. A salt of the compound having Formula (I) or (Ia), 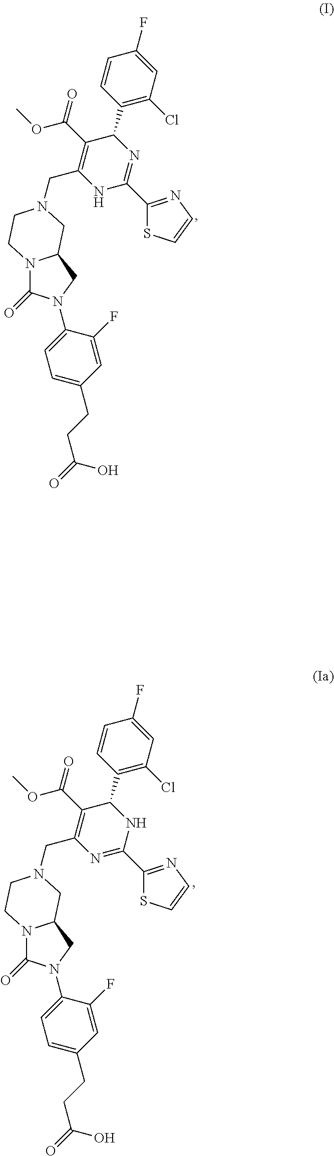
wherein, the salt is sulfate, L-arginine salt, hydrochloride, phosphate, benzenesulfonate, methanesulfonate, hydrobromide, p-toluenesulfonate or oxalate.

30. The salt according to claim 29, wherein the sulfate is sulfate crystal form B, which is characterized that the X-ray powder diffraction pattern of the sulfate crystal form B comprises diffraction peaks with 2θ angles of 6.02±0.2°, 16.74±0.2°, 17.34±0.2°, 18.17±0.2°, 19.52±0.2° and 24.32±0.2°;
which is characterized that the X-ray powder diffraction pattern of the sulfate crystal form A comprises diffraction peaks with 2θ angles of 5.74±0.2°, 8.62±0.2°, 10.52±0.2°, 13.97±0.2°, 17.75±0.2°, 19.28±0.2°, 23.38±0.2° and 24.78±0.2°;
wherein the L-arginine salt is L-arginine salt crystal form A, which is characterized that the X-ray powder diffraction pattern of the L-arginine salt crystal form A comprises diffraction peaks with 2θ angles of 10.50±0.2°, 12.52±0.2°, 16.88±0.2°, 19.30±0.2°, 20.29±0.2°, 20.61±0.2° and 23.04±0.2°;
the hydrochloride is hydrochloride crystal form A, which is characterized that the X-ray powder diffraction pattern of the hydrochloride crystal form A comprises diffraction peaks with 2θ angles of 10.94±0.2°, 11.82 0.2°, 16.64 0.2°, 19.22 0.2°, 19.64±0.2, 23.44±0.2°, 24.89±0.2° and 26.08±0.2°;
the methanesulfonate is methanesulfonate crystal form A, which is characterized that the X-ray powder diffraction pattern of the methanesulfonate crystal form A comprises diffraction peaks with 2θ angles of 5.34±0.2°, 7.82±0.2°, 14.89±0.2°, 16.62±0.2°, 19.39±0.2°, 22.41±0.2°, 23.25±0.2° and 24.08±0.2°;
the hydrobromide is hydrobromide crystal form A, which is characterized that the X-ray powder diffraction pattern of the hydrobromide crystal form A comprises diffraction peaks with 2θ angles of 6.34±0.2°, 12.03±0.2°, 15.85±0.2°, 19.67±0.2°, 21.37±0.2°, 23.33±0.2° and 25.92±0.2°;
the phosphate of the present invention is phosphate crystal form A, which is characterized that the X-ray powder diffraction pattern of the phosphate crystal form A comprises diffraction peaks with 2θ angles of 6.01±0.2°, 13.76±0.2°, 15.95±0.2°, 16.75±0.2°, 23.52±0.2°, 24.14±0.2° and 24.72±0.2°; or
the phosphate is phosphate crystal form C, which is characterized that the X-ray powder diffraction pattern of the phosphate crystal form C comprises diffraction peaks with 2θ angles of 5.44±0.2°, 6.11±0.2°, 14.67±0.2°, 15.83±0.2°, 17.35±0.2° and 19.22±0.2°.

31. The salt according to claim 29, wherein the sulfate is sulfate crystal form B, which is characterized that the X-ray powder diffraction pattern of the sulfate crystal form B comprises diffraction peaks with 2θ angles of 6.02±0.2°, 13.70±0.2°, 16.74±0.2°, 17.34±0.2, 18.17±0.2°, 19.52±0.2°, 23.72±0.2, 24.32±0.2°, 24.68±0.2° and 25.91±0.2°;
wherein the sulfate is sulfate crystal form A, which is characterized that the X-ray powder diffraction pattern of the sulfate crystal form A comprises diffraction peaks with 2θ angles of 5.74±0.2°, 8.62±0.2°, 10.52±0.2°, 13.04±0.2°, 13.97±0.2°, 17.75±0.2°, 19.28±0.2°, 23.38±0.2°, 24.78±0.2°, 25.13±0.2° and 25.76±0.2°;
wherein the L-arginine salt is L-arginine salt crystal form A, which is characterized that the X-ray powder diffraction pattern of the L-arginine salt crystal form A comprises diffraction peaks with 2θ angles of 10.50±0.2°, 12.52±0.2°, 13.52±0.2°, 16.88±0.2°, 17.07±0.2°, 19.30±0.2°, 20.29±0.2°, 20.61±0.2°, 23.04±0.2° and 28.54±0.2°;
the hydrochloride is hydrochloride crystal form A, which is characterized that the X-ray powder diffraction pattern of the hydrochloride crystal form A comprises diffraction peaks with 2θ angles of 10.94±0.2°, 11.28±0.2°, 11.82±0.2°, 12.08±0.2°, 16.64±0.2°, 19.22±0.2°, 19.64±0.2°, 20.46±0.2°, 23.44±0.2°, 24.89±0.2°, 26.08±0.2° and 28.65±0.2°;
the methanesulfonate is methanesulfonate crystal form A, which is characterized that the X-ray powder diffraction pattern of the methanesulfonate crystal form A comprises diffraction peaks with 2θ angles of 5.34±0.2°, 6.29±0.2°, 7.82±0.2°, 11.46±0.2°, 14.89±0.2°, 16.08±0.2°, 16.62±0.2°, 19.39±0.2°, 22.41±0.2°, 23.25±0.2° and 24.08±0.2°;
the hydrobromide is hydrobromide crystal form A, which is characterized that the X-ray powder diffraction pattern of the hydrobromide crystal form A comprises diffraction peaks with 2θ angles of 6.34±0.2°, 12.03±0.2°, 15.85±0.2°, 16.58±0.2°, 19.67±0.2°, 20.45±0.2°, 21.37±0.2°, 23.33±0.2°, 24.74±0.2° and 25.92±0.2°;
the phosphate of the present invention is phosphate crystal form A, which is characterized that the X-ray powder diffraction pattern of the phosphate crystal form A comprises diffraction peaks with 2θ angles of 6.01±0.2°, 12.01±0.2°, 13.07±0.2°, 13.76±0.2°, 15.95±0.2°, 16.75±0.2°, 18.11±0.2°, 23.52±0.2°, 24.14±0.2° and 24.72±0.2°; or
the phosphate is phosphate crystal form C, which is characterized that the X-ray powder diffraction pattern of the phosphate crystal form C comprises diffraction peaks with 2θ angles of 5.44±0.2°, 6.11±0.2°, 11.30±0.2°, 12.23±0.2°, 13.82±0.2°, 14.67±0.2°, 15.83±0.2°, 17.35±0.2°, 19.22±0.2° and 25.30±0.2°.

32. The salt according to claim 29, wherein the sulfate is sulfate crystal form B, which is characterized that the X-ray powder diffraction pattern of the sulfate crystal form B comprises diffraction peaks with 2θ angles of 6.02±0.2°, 9.05±0.2°, 11.28±0.2°, 12.09±0.2°, 12.68±0.2°, 13.70±0.2°, 14.17±0.2°, 15.27±0.2°, 16.29±0.2°, 16.49±0.2°, 16.74±0.2°, 17.34±0.2°, 17.56±0.2°, 18.17±0.2°, 18.69±0.2°, 19.52±0.2°, 20.47±0.2°, 21.24±0.2°, 21.87±0.2°, 22.48±0.2°, 22.71±0.2, 23.72±0.2°, 24.32±0.2°, 24.68±0.2, 24.82±0.2, 25.35±0.2°, 25.91±0.2°, 26.77±0.2, 27.36±0.2°, 27.99±0.2, 28.64±0.2°, 29.51±0.2°, 29.85±0.2°, 30.19±0.2°, 30.55±0.2°, 31.23±0.2°, 32.21±0.2°, 33.09±0.2°, 33.68±0.2°, 34.85±0.2°, 35.46±0.2°, 36.84±0.2°, 37.43±0.2°, 39.06±0.2° and 39.96±0.2°;
wherein the sulfate is sulfate crystal form A, which is characterized that the X-ray powder diffraction pattern of the sulfate crystal form A comprises diffraction peaks with 2θ angles of 5.74±0.2°, 8.62±0.2°, 10.52±0.2°, 11.08±0.2°, 13.04±0.2°, 13.97±0.2°, 14.42±0.2°, 15.40±0.2°, 16.11±0.2°, 16.56±0.2°, 17.25±0.2°, 17.75±0.2°, 18.38±0.2°, 19.28±0.2°, 19.74±0.2°, 21.14±0.2°, 21.57±0.2°, 22.33±0.2°, 23.38±0.2°, 24.78±0.2°, 25.13±0.2°, 25.76±0.2°, 26.31±0.2°, 26.80±0.2°, 27.12±0.2°, 27.83±0.2°, 28.08±0.2°, 29.32±0.2°, 30.45±0.2°, 31.31±0.2°, 31.87±0.2°, 33.08±0.2°, 34.87±0.2°, 36.01±0.2°, 36.95±0.2°, 37.42±0.2°, 38.59±0.2°, 39.03±0.2° and 39.92±0.2°;
wherein the L-arginine salt is L-arginine salt crystal form A, which is characterized that the X-ray powder diffraction pattern of the L-arginine salt crystal form A comprises diffraction peaks with 2θ angles of 8.50±0.2°, 10.50±0.2°, 12.52±0.2°, 12.71±0.2°, 13.05±0.2°, 13.52±0.2°, 14.23±0.2°, 15.76±0.2°, 16.60±0.2°, 16.88±0.2°, 17.07±0.2°, 18.22±0.2°, 19.11±0.2°, 19.30±0.2°, 19.58±0.2°, 20.29±0.2°, 20.61±0.2°, 20.98±0.2°, 22.53±0.2°, 23.04±0.2°, 24.90±0.2°, 25.41±0.2°, 25.68±0.2°, 26.11±0.2°, 26.68±0.2°, 27.22±0.2°, 28.07±0.2°, 28.29±0.2°, 28.54±0.2°, 30.12±0.2°, 31.06±0.2°, 31.68±0.2°, 33.55±0.2°, 34.50±0.2°, 34.89±0.2°, 35.24±0.2°, 36.12±0.2°, 36.65±0.2°, 38.68±0.2° and 39.80±0.2°;
the hydrochloride is hydrochloride crystal form A, which is characterized that the X-ray powder diffraction pattern of the hydrochloride crystal form A comprises diffraction peaks with 2θ angles of 10.94±0.2°, 11.28±0.2°, 11.82±0.2°, 12.08 0.2°, 12.57±0.2°, 14.06±0.2°, 15.01±0.2°, 15.81±0.2°, 16.02±0.2°, 16.64±0.2°, 17.18±0.2°, 17.86±0.2°, 18.55±0.2°, 19.22±0.2°, 19.64±0.2°, 20.46±0.2°, 21.41±0.2°, 22.19±0.2°, 23.44±0.2°, 23.85±0.2°, 24.28±0.2°, 24.89±0.2°, 25.25±0.2°, 26.08±0.2°, 26.37±0.2°, 27.09±0.2°, 27.53±0.2°, 28.00±0.2°, 28.65±0.2°, 28.91±0.2°, 30.53±0.2°, 31.42±0.2°, 31.92±0.2°, 32.40±0.2°, 33.58±0.2°, 34.36±0.2°, 35.38±0.2°, 36.07±0.2°, 37.39±0.2° and 38.58±0.2°;
the methanesulfonate is methanesulfonate crystal form A, which is characterized that the X-ray powder diffraction pattern of the methanesulfonate crystal form A comprises diffraction peaks with 2θ angles of 5.34±0.2°, 6.29±0.2°, 7.82±0.2°, 10.73±0.2°, 11.46±0.2°, 11.78±0.2°, 12.67±0.2°, 14.12±0.2°, 14.89±0.2°, 15.77±0.2°, 16.08±0.2°, 16.62±0.2°, 17.19±0.2°, 17.49±0.2°, 18.04±0.2°, 18.51±0.2°, 18.96±0.2°, 19.39±0.2°, 19.78±0.2°, 20.28±0.2°, 21.46±0.2°, 21.64±0.2°, 21.85±0.2°, 22.41±0.2°, 23.25±0.2°, 23.72±0.2°, 24.08±0.2°, 25.53±0.2°, 25.80±0.2°, 26.23±0.2°, 26.60±0.2°, 27.01±0.2°, 27.68±0.2°, 27.69±0.2°, 28.18±0.2°, 28.66±0.2°, 29.51±0.2°, 29.80±0.2°, 30.07±0.2°, 31.04±0.2°, 32.19±0.2°, 32.77±0.2°, 33.23±0.2°, 33.91±0.2°, 34.87±0.2°, 36.49±0.2°, 37.30±0.2°, 38.09±0.2°, 38.36±0.2°, 38.85±0.2°, 39.50±0.2° and 39.83±0.2°;
the hydrobromide is hydrobromide crystal form A, which is characterized that the X-ray powder diffraction pattern of the hydrobromide crystal form A comprises diffraction peaks with 2θ angles of 6.34±0.2°, 9.50±0.2°, 11.25±0.2°, 12.03±0.2°, 12.54±0.2°, 14.05±0.2°, 15.46±0.2°, 15.85±0.2°, 16.58±0.2°, 17.13±0.2°, 17.87±0.2°, 18.50±0.2°, 19.28±0.2°, 19.67±0.2°, 20.45±0.2°, 21.37±0.2°, 22.31±0.2°, 23.33±0.2°, 23.75±0.2°, 24.74±0.2°, 25.09±0.2°, 25.92±0.2°, 26.15±0.2°, 26.48±0.2°, 26.98±0.2°, 27.44±0.2°, 28.09±0.2°, 28.70±0.2°, 29.24±0.2°, 30.35±0.2°, 31.29±0.2°, 31.98±0.2°, 32.27±0.2°, 32.77±0.2°, 35.37±0.2°, 35.88±0.2°, 37.25±0.2°, 38.44±0.2° and 39.93±0.2°;
the phosphate of the present invention is phosphate crystal form A, which is characterized that the X-ray powder diffraction pattern of the phosphate crystal form A comprises diffraction peaks with 2θ angles of 6.01±0.2°, 10.88±0.2°, 12.01±0.2°, 13.07±0.2°, 13.76±0.2°, 13.88±0.2°, 14.99±0.2°, 15.64±0.2°, 15.95±0.2°, 16.75±0.2°, 18.11±0.2°, 18.37±0.2°, 18.99±0.2°, 19.76±0.2°, 20.94±0.2°, 21.16±0.2°, 21.48±0.2°, 21.78±0.2°, 22.82±0.2°, 23.52±0.2°, 24.14±0.2°, 24.72±0.2°, 25.03±0.2°, 25.63±0.2°, 25.80±0.2°, 26.34±0.2°, 26.83±0.2°, 27.15±0.2°, 28.49±0.2°, 28.90±0.2°, 29.21±0.2°, 29.61±0.2°, 30.02±0.2°, 31.55±0.2°, 32.04±0.2°, 33.37±0.2°, 33.87±0.2°, 34.36±0.2°, 35.06±0.2°, 35.42±0.2°, 35.86±0.2°, 36.53±0.2°, 36.91±0.2°, 37.67±0.2°, 38.48±0.2° and 39.91±0.2°; or
the phosphate is phosphate crystal form C, which is characterized that the X-ray powder diffraction pattern of the phosphate crystal form C comprises diffraction peaks with 2θ angles of 3.91±0.2°, 5.44±0.2°, 6.11±0.2°, 11.30±0.2°, 12.23±0.2°, 13.82±0.2°, 14.67±0.2°, 15.10±0.2°, 15.83±0.2°, 16.49±0.2°, 17.00±0.2°, 17.35±0.2°, 18.47±0.2°, 18.68±0.2°, 19.22±0.2°, 20.00±0.2°, 20.49±0.2°, 20.87±0.2°, 21.21±0.2°, 21.43±0.2°, 22.15±0.2°, 22.67±0.2°, 23.29±0.2°, 24.34±0.2°, 24.70±0.2°, 25.05±0.2°, 25.30±0.2°, 25.88±0.2°, 26.37±0.2°, 26.76±0.2°, 27.44±0.2°, 28.02±0.2°, 30.06±0.2°, 30.86±0.2°, 32.97±0.2°, 35.19±0.2°, 35.82±0.2°, 37.31±0.2°, 39.38±0.2°, 41.99±0.2°, 45.36±0.2° and 47.13±0.2°.

33. The salt according to claim 29, wherein the sulfate is sulfate crystal form B, which is characterized that the sulfate crystal form B has an X-ray powder diffraction pattern substantially as shown in FIG. 1;
the sulfate is sulfate crystal form A, which is characterized that the sulfate crystal form A has an X-ray powder diffraction pattern substantially as shown in FIG. 9;
the L-arginine salt is L-arginine salt crystal form A, which is characterized that the L-arginine salt crystal form A has an X-ray powder diffraction pattern substantially as shown in FIG. 3;
the hydrochloride is hydrochloride crystal form A, which is characterized that the hydrochloride crystal form A has an X-ray powder diffraction pattern substantially as shown in FIG. 5;
the methanesulfonate is methanesulfonate crystal form A, which is characterized that the methanesulfonate crystal form A has an X-ray powder diffraction pattern substantially as shown in FIG. 15;
the hydrobromide is hydrobromide crystal form A, which is characterized that the hydrobromide crystal form A has an X-ray powder diffraction pattern substantially as shown in FIG. 21;
the phosphate of the present invention is phosphate crystal form A, which is characterized that the phosphate crystal form A has an X-ray powder diffraction pattern substantially as shown in FIG. 11; or
the phosphate is phosphate crystal form C, which is characterized that the phosphate crystal form C has an X-ray powder diffraction pattern substantially as shown in FIG. 25.

34. The salt according to claim 29, wherein the sulfate is sulfate crystal form B, which is characterized that the differential scanning calorimetry pattern of the sulfate crystal form B comprises an endothermic peak of 227.14° C.±3° C.;
the sulfate is sulfate crystal form A, which is characterized that the differential scanning calorimetry pattern of the sulfate crystal form A comprises an endothermic peak of 208.32° C.±3° C.;
the L-arginine salt is L-arginine salt crystal form A, which is characterized that the differential scanning calorimetry pattern of the L-arginine salt crystal form A comprises an endothermic peak of 193.28° C.±3° C.;
the hydrochloride is hydrochloride crystal form A, which is characterized that the differential scanning calorimetry pattern of the hydrochloride crystal form A comprises endothermic peaks of 134.08° C.±3° C. and 176.08° C.±3° C.;
the methanesulfonate is methanesulfonate crystal form A, which is characterized that the differential scanning calorimetry pattern of the methanesulfonate crystal form A comprises endothermic peaks of 115.67° C.±3° C. and 175.40° C.±3° C.;
the hydrobromide is hydrobromide crystal form A, which is characterized that the differential scanning calorimetry pattern of the hydrobromide crystal form A comprises endothermic peaks of 120.25° C.±3° C. and 194.76° C.±3° C.;
the phosphate of the present invention is phosphate crystal form A, which is characterized that the differential scanning calorimetry pattern of the phosphate crystal form A comprises an endothermic peak of 145.36° C.±3° C.; or
the phosphate is phosphate crystal form C, which is characterized that the differential scanning calorimetry pattern of the phosphate crystal form C comprises an endothermic peak of 172.9° C.±3° C.

35. The salt according to claim 29, wherein the sulfate is sulfate crystal form B, which is characterized that the sulfate crystal form B has a differential scanning calorimetry pattern substantially as shown in FIG. 2;
the sulfate is sulfate crystal form A, which is characterized that the sulfate crystal form A has a differential scanning calorimetry pattern substantially as shown in FIG. 10;
the L-arginine salt is L-arginine salt crystal form A, which is characterized that the L-arginine salt crystal form A has a differential scanning calorimetry pattern substantially as shown in FIG. 4;
the hydrochloride is hydrochloride crystal form A, which is characterized that the hydrochloride crystal form A has a differential scanning calorimetry pattern substantially as shown in FIG. 6;
the methanesulfonate is methanesulfonate crystal form A, which is characterized that the methanesulfonate crystal form A has a differential scanning calorimetry pattern substantially as shown in FIG. 16;
the hydrobromide is hydrobromide crystal form A, which is characterized that the hydrobromide crystal form A has a differential scanning calorimetry pattern substantially as shown in FIG. 22;
the phosphate of the present invention is phosphate crystal form A, which is characterized that the phosphate crystal form A has a differential scanning calorimetry pattern substantially as shown in FIG. 12; or
the phosphate is phosphate crystal form C, which is characterized that the phosphate crystal form C has a differential scanning calorimetry pattern substantially as shown in FIG. 26.

36. A N,N-dimethylformamide complex of the compound having Formula (I) or Formula (Ia), 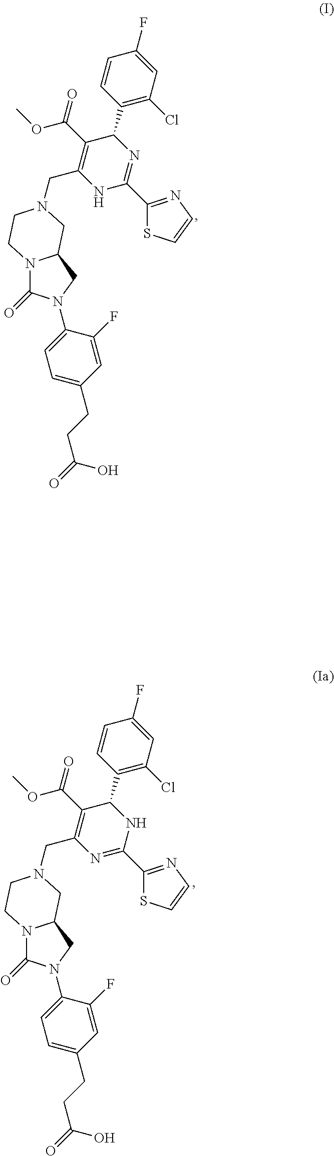 [SMILES: COC(=O)C1=C(CN2CCN3C(=O)N(c4ccc(CCC(=O)O)cc4F)C[C@@H]3C2)NC(c2nccs2)=N[C@H]1c1ccc(F)cc1Cl ; COC(=O)C1=C(CN2CCN3C(=O)N(c4ccc(CCC(=O)O)cc4F)C[C@@H]3C2)N=C(c2nccs2)N[C@H]1c1ccc(F)cc1Cl]
wherein the X-ray powder diffraction pattern of the N,N-dimethylformamide complex comprises diffraction peaks with 2θ angles of 10.31±0.2°, 10.91±0.2°, 17.04±0.2°, 19.18±0.2°, 20.17±0.2°, 21.83±0.2° and 24.41±0.2°; or
wherein the X-ray powder diffraction pattern of the N,N-dimethylformamide complex comprises diffraction peaks with 2θ angles of 6.30±0.2°, 10.31±0.2°, 10.91±0.2°, 14.89±0.2°, 16.54±0.2°, 17.04±0.2°, 19.18±0.2°, 20.17±0.2°, 21.83±0.2° and 24.41±0.2°.

37. The N,N-dimethylformamide complex according to claim 36, wherein the X-ray powder diffraction pattern of the N,N-dimethylformamide complex comprises diffraction peaks with 2θ angles of 6.30±0.2°, 7.19±0.2°, 8.85±0.2°, 10.31±0.2°, 10.91±0.2°, 11.36±0.2°, 11.93±0.2°, 12.53±0.2°, 12.93±0.2°, 13.93±0.2°, 14.89±0.2°, 15.31±0.2°, 15.90±0.2°, 16.54±0.2°, 17.04±0.2°, 17.94±0.2°, 18.39±0.2°, 18.69±0.2°, 19.18±0.2°, 20.17±0.2°, 20.70±0.2°, 20.96±0.2°, 21.60±0.2°, 21.83±0.2°, 22.18±0.2°, 22.49±0.2°, 22.74±0.2°, 23.37±0.2°, 23.77±0.2°, 24.41±0.2°, 24.70±0.2°, 25.13±0.2°, 25.71±0.2°, 26.14±0.2°, 26.45±0.2°, 27.44±0.2°, 28.02±0.2°, 28.30±0.2°, 28.76±0.2°, 29.52±0.2°, 30.12±0.2°, 30.68±0.2°, 31.18±0.2°, 31.66±0.2°, 31.98±0.2°, 33.24±0.2°, 33.82±0.2°, 34.44±0.2°, 34.76±0.2°, 36.00±0.2°, 37.34±0.2°, 37.83±0.2°, 38.92±0.2° and 39.61±0.2°.

38. The N,N-dimethylformamide complex according to claim 36, wherein the N,N-dimethylformamide complex has an X-ray powder diffraction pattern substantially as shown in FIG. 7; or
the N,N-dimethylformamide complex has a differential scanning calorimetry pattern substantially as shown in FIG. 8; or
wherein the differential scanning calorimetry pattern of the N,N-dimethylformamide complex comprises an endothermic peak of 120.20° C.±3° C.

39. A pharmaceutical composition comprising the salt of claim 29, and a pharmaceutically acceptable carrier, excipient, diluent, adjuvant, vehicle or a combination thereof.

40. A pharmaceutical composition comprising the N,N-dimethylformamide complex of claim 36, and a pharmaceutically acceptable carrier, excipient, diluent, adjuvant, vehicle or a combination thereof.

41. A method of preventing, managing, treating or lessening viral disease in a patient comprising administering to the patient a therapeutically effective amount of the salt of claim 29, wherein the viral disease is a hepatitis B virus infection or a disease caused by hepatitis B virus infection, wherein the disease caused by hepatitis B virus infection is liver cirrhosis or hepatocellular carcinoma.

42. A method of preventing, managing, treating or lessening viral disease in a patient comprising administering to the patient a therapeutically effective amount of the N,N-dimethylformamide complex of claim 36, wherein the viral disease is a hepatitis B virus infection or a disease caused by hepatitis B virus infection, wherein the disease caused by hepatitis B virus infection is liver cirrhosis or hepatocellular carcinoma.

43. A method of preventing, managing, treating or lessening viral disease in a patient comprising administering to the patient a therapeutically effective amount of the pharmaceutical composition of claim 39, wherein the viral disease is a hepatitis B virus infection or a disease caused by hepatitis B virus infection, wherein the disease caused by hepatitis B virus infection is liver cirrhosis or hepatocellular carcinoma.

44. A method of preventing, managing, treating or lessening viral disease in a patient comprising administering to the patient a therapeutically effective amount of the pharmaceutical composition of claim 40, wherein the viral disease is a hepatitis B virus infection or a disease caused by hepatitis B virus infection, wherein the disease caused by hepatitis B virus infection is liver cirrhosis or hepatocellular carcinoma.
